(19) United States
(12) Patent Application Publication
(10) Pub. No.: US 2011/0294873 A1
(43) Pub. Date: Dec. 1, 2011

(54) Gene Transfer Vectors Comprising At Least One Isolated DNA Molecule Having Insulator Or Boundary Properties And Methods To Identify The Same

(75) Inventors: Nicolas Mermod, Buchillon (CH); Armelle Gaussin, Lausanne (CH); Germain Esnault, Renens (CH)

(73) Assignee: UNIVERSITÉ DE LAUSANNE, Lausanne (CH)

(21) Appl. No.: 13/125,750

(22) PCT Filed: Oct. 23, 2009
(86) PCT No.: PCT/EP09/64023

§ 371 Date: Apr. 22, 2011

Related U.S. Application Data
(60) Provisional application No. 61/193,035, filed on Oct. 23, 2008.
(60) Provisional application No. 61/202,299, filed on Feb. 17, 2009.

(51) Int. Cl. A61K 48/00; C07H 21/04; C12N 5/10; C12N 15/63; C12Q 1/68

(52) U.S. Cl. 514/44 R; 435/320.1; 435/6.13; 435/6.12; 435/365; 435/358; 435/325; 435/367; 435/366; 536/24.1; 435/354

(57) ABSTRACT

The present invention relates to gene transfer vectors and in particular expression vectors which comprise at least one isolated DNA molecule having insulator and or boundary properties which limits the effects of a regulatory sequence upon another regulatory or coding sequence disposed upon the other side of said at least one isolated DNA molecule. The present invention also relates to methods of identifying isolated DNA molecule having insulator and or boundary properties and to the use of expression vector, in particular a retrovirus vector, in in vivo and ex vivo gene therapy methods as well as to cells and organisms transformed using vectors according to the present invention.

### FIELD OF THE INVENTION

The present invention relates to gene transfer vectors and in particular expression vectors which comprise at least one isolated DNA molecule having insulator and or boundary properties which limits the effects of a regulatory sequence upon another regulatory or coding sequence disposed upon the other side of said at least one isolated DNA molecule. The present invention also relates to methods of identifying isolated DNA molecule having insulator and or boundary properties and to the use of expression vector, in particular a retrovirus vector, in in vivo and ex vivo gene therapy methods as well as to cells and organisms transformed using vectors according to the present invention.

### BACKGROUND ART

The need for better gene transfer systems, in terms of specificity, efficacy and safety has been and still remains a major challenge in the development of gene therapy, so that the risk/benefit balance of treating a condition via a gene therapy method will be improved sufficiently to allow the routine use of gene therapy in patients.

Retroviral (RV) and other viral and non-viral vectors used in gene therapy often have a preference for particular chromosomal integration regions or targets. It is also well known that chromosomal insertion of a vector can activate or indeed inactivate genes nearby on the chromosome and that chromosomal regulatory sequences can affect the expression of vector encoded genes, this phenomenon being known as regulatory cross talk. When endogenous genes are improperly expressed in this way, this can lead to these cells becoming cancerous. This oncogenic potential of vectors may stem from the promiscuous activation of cellular genes by endogenous viral regulatory elements and/or exogenous regulatory elements which for instance drive the expression of the exogeneous therapeutic gene product present in the vector. In otherwise successful gene therapy trials, these types of effects have resulted in otherwise unexplained cases of spontaneous leukemia and death in some of the patients.

These major secondary effects have been reported in gene therapy trials in patients with X-linked severe combined immunodeficiency (SCID-X1) with five reported cases of vector induced leukaemia found both in the Paris & London gene therapy trials. Similar effects have also been seen in several other gene therapy trials, for instance X-linked chronic granulomatous disease (X-CGD). These observed side effects of integrative vector mediated gene therapy, reveal the limitations of integrative vectors for gene transfer (Cavazzana-Calvo et al, 2000 and Hacein-Bey-Albina et al, 2003) currently under use in clinical studies.

It has been shown that the integration of murine leukemia virus (MLV) based vectors in the SCID-X1 patient was not random. Integration occurred mainly within or close to specific regions of genes. Insertional mutagenesis can result in acute toxicity (i.e. loss of transduced cells due to mutations of essential genes) or delayed side effects such as cancer induction. The nature and pathogenicity of these delayed side effects are highly context-dependent and will depend in part upon differences in the type of vector, transgene cassette, target cell, transduction conditions (copy number per cell) and disease-specific in vivo conditions for the maintenance and expansion of gene-modified cells.

Most experiments/clinical trials performed so far with insulated retroviral vectors incorporate either the 1200 bp long HS4 insulator from chicken beta globin or a 250 bp long core sequence from this insulator as single or double copy cloned into the virus LTR (Ye et al., (2003)). It has now been shown that the 1200 bp HS4 insulator is not genetically stable in viral constructs; and it has also been established that the core sequence when present in one or two copies does not shield adjascent genomic neighbouring sequences against unwanted activation by the enhancer/promoter combination driving transgene expression.

There is thus a need to identify alternative sequences which both have enhancer-blocking activity and which have a boundary effect, as well as being stable in the virus and having no major effects upon virus biology and replication.

The present invention therefore relates to a new class of expression vectors, which comprise in the nucleotide sequence to be integrated into the genome, an isolated DNA molecule having insulator and or boundary properties which prevents or significantly lessens the effects of the integrated sequences upon genomic sequences and vice versa the effects of genomic sequences upon the integrated sequence.

Furthermore, in order to decrease the risk associated with the use of viral vectors, the present invention proposes to identify genetic insulators/boundaries capable of isolating the vector regulatory elements. These insulator/boundary elements can be integrated into retroviral vectors and/or other viral vectors to prevent the activation of chromosomal genes by the viral enhancers and do not interfere with therapeutic effects.

### SUMMARY OF THE INVENTION

According to a first aspect of the present invention there is provided an isolated DNA molecule having insulator and or boundary properties. Said isolated DNA molecule comprises at least one binding site sequence for a protein selected from the group consisting of CTF/NF-1, a CTCF construct, a combination of CTF/NF-1 and CTCF, a combination of CTF/NF-1 and a CTCF construct and/or combinations thereof.

Another object of the present invention is to provide for an expression vector comprising
(a) at least one copy of said isolated DNA molecule having insulator and or boundary properties,
(b) a promoter domain;
(c) a gene of interest operably linked to the promoter domain, and
(d) an enhancer domain 5′ of the promoter domain,

A further object of the invention is to provide for a method of detecting a DNA molecule having insulator and/or boundary properties comprising the steps of:

a) providing an expression vector wherein said isolated DNA molecule is positioned between a potent enhancer and a promoter domain operably linked to a reporter gene,
b) introducing the expression vector of step a) into a cell,
c) quantifying the expression of the reporter gene, and
d) correlating said reporter gene expression to potential insulator or boundary properties of said DNA molecule.

Also encompassed is the isolated DNA molecule having insulator or boundary properties identified according to the above identified method.

A yet further object of the invention is to provide for a method for treating a subject diagnosed with a genetic disease, the method comprising administering an expression vector of the invention so as to complement the genetic deficiency.

Also provided is a host cell comprising the isolated DNA molecule according to the invention and/or at least one copy of the expression vector of the invention.

In addition the present invention also concerns a mammalian cell stably transfected with the isolated DNA molecule of the invention and/or at least one copy of the expression vector of the invention.

### BRIEF DESCRIPTION OF THE DRAWINGS

For a better understanding of the invention and to show how the same may be carried into effect, there will now be shown by way of example only, specific embodiments, methods and processes according to the present invention with reference to the accompanying drawings in which:

FIG. 1. Gal-Pro protects transgenes from telomeric position silencing effects.

FIG. 2. Specific boundary activity of Gal-Pro at telomeric transgenes.

FIG. 3. Native CTF I acts as boundary at human cell telomeres.

FIG. 4. Telomeric histones H3 and H4 are hypoacetylated.

FIG. 5. Effect of the Gal-Pro boundary on telomeric chromatin structure.

FIG. 6. Localization of telomeric transgene integration in four stable cells clones.

FIG. 7. Pattern of expression of DsRed and GFP for the 12 monoclonal populations selected from stable transfections.

FIG. 8. Non-targeted transgene integration in clones generated without telomeric repeats.

FIG. 9. The boundary activity of Gal-Pro at telomeric loci depends on the relative position but not on the reporter gene identity.

FIG. 10. Deletions of the H3-interacting domains of CTF1 abolish its telomeric boundary activity.

FIG. 11. Point mutations in CTF1 H3-interacting domains inhibit the boundary activity.

FIG. 12. Telomeric position silencing effects are relieved by histone deacetylation inhibitors.

FIG. 13: Design of a plasmid-based screening procedure for potential insulator elements in parallel with gene transfer-mediated insertional activation

FIG. 14: First generation assay system plasmids

FIG. 15: Comparison of the cHS4 insulator effect in HeLa and K562 cells

FIG. 16: Evaluation of cHS4 ability to insulate the Fr-MLV LTR enhancer in HeLa cells

FIG. 17: FACS analysis of the cHS4 insulator effect in an enhancer blocking assay

FIG. 18: Semi-quantitative analysis of the cHS4 insulator effect

FIG. 19: Semi-quantitative analysis of insulator effect

FIG. 20. Assessment of the insulator activity of multimerized CTF binding sites using the commonly-used neomycin-resistance insulator assay.

FIG. 21: Schematic illustration of the improved plasmid-based screening assay for potential insulator elements.

FIG. 22: Comparison of FACS profiles of BFP and GFP expression levels of HeLa cells transfected with the improved insulator assay constructs either with or without the cHS4.

FIG. 23: Quantitative analysis of CTCF binding sites insulator activity compared to the cHS4.

FIG. 24: Quantitative analysis of Ins2 binding sites insulator activity compared to the cHS4.

FIG. 25: Description of Ins2 binding site derivatives

FIG. 26: Schematic diagrams of insulator/enhancer-blocker assay systems and reporter genes expression analysis

FIG. 26(A): Schematic representation of the vectors used for the insulator assay based on the quantitation of neomycin-resistant colonies.

FIG. 26(B): Schematic illustration of the quantitative assay for enhancer-blockers.

FIG. 26(C): Percentage of neomycin-resistant colonies counted 2 to 3 weeks after transfection and G418 selection of HeLa (dashed bars) and K562 (black bars) cells.

FIG. 26(D): Cytofluorometric analysis of the cHS4 insulator activity using the quantitative assay in transiently transfected HeLa cells.

FIG. 26(E): Quantitative analysis of the cHS4 insulator enhancer-blocking activity.

FIG. 27: Quantitative analysis of CTF/NFI binding sites enhancer-blocking activity compared to the cHS4

FIG. 27(A): Sequence description and pairwise alignment of the different types of CTF/NFI binding sites constructed and assessed.

FIG. 27(B): Quantitative analysis of the enhancer-blocking activity of CTF/NFI binding sites.

FIG. 27(C): Quantitative analysis of the enhancer-blocking activity of CTF/NFI binding sites.

FIG. 27(D): Quantitative analysis of the enhancer-blocking activity of CTF/NFI binding sites in stable transfections.

FIG. 28: CTF/NFI proteins mediate the enhancer-blocking activity of cognate DNA binding sites

FIG. 28(A): Quantitative analysis of the enhancer-blocking properties of CTF/NFI binding sites.

FIG. 28(B): Western-blot analysis of the cell populations analyzed in panel A.

FIG. 29: CTF/NFI binding sites dampen chromosomal position-effect

FIG. 29(A): Schematic representation of the insulated GFP transgene.

FIG. 29(B): Results of representative FACS analysis for GFP expression of HeLa cell populations stably transfected with constructs described in panel A (16 days after 0 transfection).

FIG. 29(C): Relative distribution of each sub-population of cells according to GFP expression levels.

FIG. 29(D): Time course FACS analysis of the GFP transgene expression when flanked with various insulators in stably transfected HeLa cells.

FIG. 30: CTF/NF1 and CTCF binding sites decrease the genotoxicity or retroviral vectors

FIG. 30A: Vector architecture of the gammaretroviral self-inactivating (SIN) vector SRS.SF.eGFP.pre shown as provirus.

FIG. 30 B: The insulator sequences into the LTRs of the SRS.SF.eGFP.pre vectors reduced its transformation potential

FIG. 30 C: Quantitative real-time PCR analysis

### BRIEF DESCRIPTION OF THE SEQUENCES

SEQ ID 1: This sequence represent CTF/NF1 binding site from the adenovirus type II origin of replication

SEQ ID 2: This sequence represent binding site for CTCF from the footprint II (FII) of the cHS4 insulator

SEQ ID 3: This sequence represent Binding site for CTCF from the human T cell receptor alpha/delta locus BEAD A

SEQ ID 4-5: This sequence is complementary to the murine GAPDH cDNA for quantitative PCR

SEQ ID 6-7: This is sequence complementary to the EGFP cDNA for quantitative PCR

SEQ ID 8-9: This Sequence is complementary to the dsRED cDNA for quantitative PCR

SEQ ID 10: This sequence represents Murine telomeric repeat

SEQ ID 11:This sequence represents 12×CTCF

SEQ ID 12: This sequence represents consensus binding sites for CTCF.

SEQ ID 13: This sequence represents 1×CTF/NF1 from adenovirus type II

SEQ ID 14: This sequence represents 7×CTF/NF1 from adenovirus type II

SEQ ID 15: This sequence represents 3×CTF/NF1 from adenovirus type II but combination of sites and flanking sequences artificial

SEQ ID 16: This sequences represents 7×CTF/NF1 from adenovirus type II but combination of sites and flanking sequences artificial

SEQ ID 17: This sequence represents 3×CTF/NF1

SEQ ID 18: This sequence represents 7×CTF/NF1

SEQ ID 19: This sequence represents 3×CTF/NF1

SEQ ID 20: This sequence represents 7×CTF/NF1

SEQ ID 21: This sequence represents 1×CTCF consensus sequence

SEQ ID 22: This sequence represents 4 CTF/NF1 binding sites

SEQ ID 23: This sequence represents 4 CTF/NF1 binding sites

SEQ ID 24: This sequence represents 1 CTF/NF1 binding site

SEQ ID 25: This sequence represents 3 CTF/NF1 binding sites

SEQ ID 26: This sequence represents 3 CTF/NF1 binding sites

SEQ ID 27: This sequence represents 4 CTF/NF1 binding sites

SEQ ID 28: This sequence represents 4 CTF/NF1 binding sites

SEQ ID 29: This sequence represents 1 CTF/NF I consensus

SEQ ID 30: This sequence represents 1×CTCF consensus sequence (complementary to SEQ ID No 21)

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

As used herein, the following definitions are supplied in order to facilitate the understanding of the present invention.

The term “comprise” is generally used in the sense of include, that is to say permitting the presence of one or more features or components.

The present invention provides newly-identified insulator nucleic acid sequences that act as a barrier to the influences of neighboring cis and/or trans-acting elements, thereby preventing gene activation, for example, when juxtaposed between an enhancer sequence and a promoter sequence.

The newly-characterized and isolated insulator elements (DNA molecules) of the invention are able to insulate or buffer the expression of a reporter gene from adverse effects of neighboring or surrounding chromatin. The incorporation of the defined insulator sequence into vectors and constructs allows gene transfer and expression in cells and tissues with virtually no concern for suppression or inhibition of expression due to the chromosomal milieu after integration.

“Insulators and boundaries” are DNA elements that may alter gene expression by preventing activation or inhibitory effects that stem from their chromosomal environment (4, 43). Insulators and boundaries are often defined as DNA elements that can prevent the action of an enhancer or silencer on a promoter when interposed between the promoter and the regulatory sequence. Chromatin domain boundaries are defined as elements that prevent the propagation of chromatin features, such as hetero chromatin, and they may thereby demarcate chromosomal domains that possess distinct chromatin features and gene expression status”. Insulators and boundaries are typically capable of both blocking enhancer activity and protecting against position effects. These two functions might have only partially overlapping mechanisms. Protection against position effects implies that activation by external endogenous enhancers is blocked, consistent with the activity described herein. However, position effects also arise from silencing induced by neighboring heterochromatin. While the insulators and/or boundaries described herein are able to protect against external position effects, it may also be that additional components of the insulators and/or boundaries elements, or additional cofactors, are involved in protecting against such effects.

According to the present invention it is provided an isolated DNA molecule having insulator and or boundary properties wherein said isolated DNA molecule having insulator and or boundary properties comprises at least one binding site sequence for a protein selected from the group consisting of CTF/NF-1, a CTCF construct. Note that CTF/NF-1 (also called NF1, NFI, CTF, NF1/CTF, NFI/CTF in the litterature) indicates a familly of proteins that bind highly similar or identical DNA sequences (Rupp et al., (1990); Gronostajski, R. M. 2000. Roles of the NFI/CTF gene family in transcription and development. Gene 249:31-45). The family is composed of 4 subfamilies of proteins encoded by 4 distinct genes (NF-1A, NF-1B, NF-1C and NF-1X). NF-1C is also called CTF, and individual polypeptides such as NF-1C1, NF-1C2, etc, were originally called CTF-1, CTF-2, etc. Given the complexity of the nomenclature, members of the family will be called indifferently CTF/NF-1, NF1 or CTF in the following text and figures.

The insulator and/or boundary defined herein is a DNA molecule which is capable of acting as a barrier to neighboring cis or trans-acting elements, insulating the transcription of a gene placed within its range of action, when juxtaposed between an enhancer and a promoter. Gene activation by external endogenous enhancers is blocked when the insulator is positioned between the enhancer and the promoter of a given gene.

A DNA molecule encompassed by the present invention might be any polydeoxynucleotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid.

“A purified and isolated DNA molecule or sequence” refers to the state in which the nucleic acid molecule is free or substantially free of material with which it is naturally associated such as other polypeptides or nucleic acids with which it is found in its natural environment, or the environment in which it is prepared (e.g. cell culture) when such preparation is by recombinant nucleic acid technology practiced in vitro or in vivo.

Preferably CTF/NF1 is a CTF (or NF-IC) and even more preferably CTF is a CTF1 (or NFI-C1).

In particular, CTCF construct comprises a sequence selected from the group consisting of SEQ ID No 2, SEQ ID No 3, SEQ ID No 11, SEQ ID No 12, SEQ ID No 21, SEQ ID No 30 and/or combinations thereof.

According to one embodiment of the invention, the CTF binding site comprises a sequence selected from the group consisting of SEQ ID No 1, SEQ ID No 13, SEQ ID No 14, SEQ ID No 15, SEQ ID No 16, SEQ ID No 17, SEQ ID No 18, SEQ ID No 19, SEQ ID No 20, SEQ ID No 22, SEQ ID No 23, SEQ ID No 24, SEQ ID No 25, SEQ ID No 26, SEQ ID No 27, SEQ ID No 28, SEQ ID No 29 and/or combinations thereof.

In addition, the isolated DNA molecule with insulator or boundary propertiesis characterized in that, it comprises at leastone binding site sequence element for a protein selected from the group consisting of USF1/2, BRCA1, Oct1, Sp1, Ars1, SatB1, CREB, C/EBP, NMP4, Hox, Gsh, Fast 1, biologically active fragments thereof, variants thereof, and/or combinations thereof.

With “variants” or “variants of a sequence” is meant a nucleic acid sequence that vary form the reference sequence by conservative nucleic acid substitutions, whereby one or more nucleic acids are substituted by another with same characteristics. Variants encompass as well degenerated sequences, sequences with deletions and insertions, as long as such modified sequences exhibit the same function (functionally equivalent) as the reference sequence.

“Fragments” refer to sequences sharing at least 40% amino acids in length with the respective sequence of the substrate active site. These sequences can be used as long as they exhibit the same biological properties as the native sequence from which they derive. Preferably these sequences share more than 70%, preferably more than 80%, in particular more than 90%, and even more than 95% amino acids in length with the respective sequence the substrate active site. These fragments can be prepared by a variety of methods and techniques known in the art such as for example chemical synthesis.

The present invention also includes variants of the aforementioned sequences, that is nucleotide sequences that vary from the reference sequence by conservative nucleotide substitutions, whereby one or more nucleotides are substituted by another with same characteristics. Variants encompass as well degenerated sequences, sequences with deletions and insertions, as long as such modified sequences exhibit the same biological function (functionally equivalent) as the reference sequence.

Molecular chimera of the aforementioned sequences, are also considered in the present invention. By molecular chimera is intended a nucleotide sequence that may include a functional portion of the isolated DNA molecule according to the invention and that will be obtained by molecular biology methods known by those skilled in the art.

Particular combinations of isolated DNA molecules or fragments or sub-portions thereof are also considered in the present invention. These fragments can be prepared by a variety of methods known in the art. These methods include, but are not limited to, digestion with restriction enzymes and recovery of the fragments, chemical synthesis or polymerase chain reactions (PCR).

Usually, the isolated DNA molecule having insulator and or boundary properties is a combination of one or more of the aforementioned sequences. Preferably, the combination consists in a combination of two, three, four, five, six, or seven of the aforementioned sequences.

Usually, the isolated DNA molecule according to the invention is a combination of one or more SEQ ID No 1. Preferably, the combination consists in a combination of seven SEQ ID No 1.

According to another embodiment of the invention, the isolated DNA molecule consists in a combination of one or more SEQ ID No 2. Preferably, the combination consists in a combination of six SEQ ID No 2.

Usually, the isolated DNA molecule having insulator and or boundary properties is a combination of one or more of the aforementioned sequences. Preferably, the combination consists in a combination of two, three, four, five, six, seven, eight or even twelve of the aforementioned sequences.

As described above, the isolated DNA molecule having insulator and or boundary properties of the invention has enhancer-blocking and/or a boundary function.

CCCTC-binding factor (CTCF) is a well known regulatory protein whose function in various regulatory and developmental pathways continues to be elucidated (Ohlsson et al., (2001) and (Tae et al., (2007)) and consensus binding site sequences have been proposed (Tae et al., (2007)). The inventors have shown that by combining consensus CTCF binding sites, novel enhancer-blocker elements can be generated. Unexpectedly, it is shown that 12 copies of this sequence is as potent as the complete 1.2 kb cHS4 element, and yet it is much shorter.

As described above, a significant advantage of the insulator sequences defined by aforementioned sequences is that they are small molecules and are more versatile for use in a variety of vectors for gene delivery into cells and organisms. By contrast, the larger insulators/boundaries are cumbersome and their sizes may preclude their use in some applications of gene delivery and/or gene transfer. Indeed, according to the insulators/boundaries of the present invention have been found to be both necessary and sufficient for insulating and enhancer-blocking effects and so may be preferentially used as insulators/boundaries of choice in the vectors and constructs embraced by the present invention.

Another aspect of the insulator and/or boundary sequence described herein, or the insulator bound by its cognate DNA binding protein, is the protection of a stably integrated reporter gene from position effects.

According to the present invention, the insulator element or the isolated DNA molecule of the invention is preferably located between an enhancer and a promoter to influence expression. The position of the insulator/boundary is the determining factor; it can be inserted in either orientation with equal effect and insulator/boundary function.

In a preferred aspect of the present invention, the insulator element is situated between the enhancer and the promoter of a given gene to buffer the effects of a cis-acting DNA region on the promoter of the transcription unit. In some cases, the insulator can be placed distantly from the transcription unit. In addition, the optimal location of the insulator element can be determined by routine experimentation for any particular DNA construct. The function of the insulator element is substantially independent of its orientation, and thus the insulator element can function when placed in genomic or reverse genomic orientation with respect to the transcription unit to insulate the gene from the effects of cis-acting DNA sequences of chromatin.

Preferably, the isolated DNA molecule of the invention is functionally linked to a U3-deleted LTR.

The term “functionally or operably linked” refers to a juxtaposition wherein the components are in a relationship permitting them to function in their intended manner (e.g. functionally linked).

As used herein, the term “promoter” refers to a nucleic acid sequence that regulates expression of a gene. A promoter sequence is a DNA regulatory region capable of binding RNA polymerase in a cell and initiating transcription of a downstream (3′ direction) coding sequence. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine Dalgarno sequences in addition to the −10 and −35 consensus sequences.

A “hybrid promoter” as used herein refers to a promoter comprising two or more regulatory regions or domains, which are from different origins, i.e. which do not occur together in the nature.

An “enhancer” is a nucleotide acid sequence that acts to potentiate the transcription of genes independent of the identity of the gene, the position of the sequence in relation to the gene, or the orientation of the sequence.

In accordance with the invention, the insulator element, reporter gene (s), and transcription unit may be provided in the form of a cassette designed to be conveniently ligated into a suitable plasmid or vector, which plasmid or vector is then used to transfect cells or tissues, and the like, for both in vitro and in vivo use.

In accordance with another object of the present invention there is provided an expression vector comprising:
(a) at least one copy of the isolated DNA molecule (insulator element) as described above,
(b) a promoter domain;
(c) a gene of interest operably linked to the promoter domain, and
(d) an exogenous enhancer domain 5′ of the promoter domain.

The terms “vector” and “plasmid” are used interchangeably, as the plasmid is the most commonly used vector form. However, the invention is intended to include such other forms of expression vectors, including, but not limited to, viral vectors (e.g., retroviruses (including lentiviruses), adenoviruses and adeno-associated viruses), which serve equivalent functions. Preferably, the expression vector according to the invention is a retroviral expression vector.

The expression vector of the invention can be in the form of a linear or a circular DNA sequence. “Linear DNA” denotes non-circular DNA molecules having free 5′ and 3′ ends. Linear DNA can be prepared from closed circular DNA molecules, such as plasmids, by enzymatic digestion or physical disruption. “Circular DNA” denotes non-circular DNA molecules having free 5′ and 3′ ends. Linear DNA can be prepared from closed circular DNA molecules, such as plasmids, by enzymatic digestion or physical disruption.

The vectors or constructs as used herein broadly encompass any recombinant DNA material that is capable of transferring DNA from one cell to another. The vector as described in the above embodiment can represent a minilocus which can be integrated into a mammalian cell where it can replicate and function in a host cell type-restricted and copy number dependent manner, independent of the site of integration. Thus, the expression and production of the introduced gene is insulated from any effects exerted by neighboring genetic loci or chromatin following integration.

Those skilled in the art will appreciate that a variety of enhancers, promoters, and genes are suitable for use in the constructs of the invention, and that the constructs will contain the necessary start, termination, and control sequences for proper transcription and processing of the gene of interest when the construct is introduced into a vertebrate cell, such as that of mammal or a higher eukaryote. The constructs may be introduced into cells by a variety of gene transfer methods known to those skilled in the art, for example, gene transfection, lipofection, microinjection, electroporation, transduction and infection. In addition, it is envisioned that the invention can encompass all or a portion of a viral sequence containing vector, such as those described in U.S. Pat. No. 5,112,767, as known to those skilled in the art, for targeted delivery of genes to specific tissues. It is preferred that the constructs of the invention integrate stably into the genome of specific and targeted cell types.

More preferably, said at least one copy of the isolated DNA molecule is positioned between the enhancer and the promoter domains so as to operably insulate the transcription and expression of the gene from cis-acting regulatory elements in chromatin. Even more preferably, said at least one copy of the isolated DNA molecule substitues a part of the said expression vector and said expression vector is a self-inactivating vector following insertion into the genome.

As described above, the expression vector of the invention comprises between one and twelve copies of said isolated DNA molecule according to present invention.

Preferably said at least one copy of the isolated DNA molecule comprises between two and twelve copies of said CTF binding site.

The expression vector of the invention may further comprise a gene of interest.

A “gene” is a deoxyribonucleotide (DNA) sequence coding for a given mature protein. As used herein, the term “gene” shall not include untranslated flanking regions such as RNA transcription initiation signals, polyadenylation addition sites, promoters or enhancers.

The “gene of interest” or “transgene” is preferably a gene which encodes a protein (structural or regulatory protein). The proteins may be “homologous” to the host (i.e., endogenous to the host cell being utilized), or “heterologous,” (i.e., foreign to the host cell being utilized), such as a human protein produced by yeast. The protein may be produced as an insoluble aggregate or as a soluble protein in the periplasmic space or cytoplasm of the cell, or in the extracellular medium. Examples of proteins include antibodies, hormones such as growth hormone, growth factors such as epidermal growth factor, analgesic substances like enkephalin, enzymes like chymotrypsin, and receptors to hormones or growth factors and includes as well proteins usually used as a visualizing marker e.g. green fluorescent protein.

The gene of interest may also code for an antisense molecule whose transcription in a host cell enables gene expression of the transcription of cellular mRNAs to be controlled. Such molecules can, for example, be transcribed in a host cell into RNAs complementary to cellular mRNAs and thus block their translation into protein, according to techniques known in the art.

The gene of interest may also code for a polypeptide of diagnostic use or therapeutic use. The polypeptide may be produced in bioreactors in vitro using various host cells (e.g., COS cells or CHO cells or derivatives thereof) containing the expression vector of the invention.

The gene of interest may also code for an antigenic polypeptide for use as a vaccine. Antigenic polypeptides or nucleic acid molecules are derived form pathogenic organisms such as, for example, a bacterium or a virus.

In case the gene of interest is supposed to be exported by transfected cells, the expression vector of the invention can further comprise a peptide signal sequence. “Signal sequence” refers to a polynucleotide sequence which encodes a short amino acid sequence (i.e., signal peptide) present at the NH2-terminus of certain proteins that are normally exported by cells to noncytoplasmic locations (e.g., secretion) or to be membrane components. Signal peptides direct the transport of proteins from the cytoplasm to noncytoplasmic locations. One skilled in the art would easily identify such signal sequences.

In particular the expression vector is a retroviral expression vector.

“Retroviral vectors” are based upon retroviruses, this group of viruses has a very characterisitic and well-known genomic structure comprising at either end of the linear DNA genome, (that is the genome produced by reverse transcription of the RNA genome), this comprises two LTR regions which each comprise a U3, R and U5 regions in that order. Contained between these LTR regions are the coding and regulatory sequences of the retrovirus and it is into this central portion of the retroviral genome that sequences encoding therapeutic gene products are inserted.

In particular the retrovirus vector is a gammaretrovirus or lentivirus vector.

Further examples of retroviruses include: avian leukosis-sarcoma, mammalian C-type, B-type viruses, D type viruses, HTLV-BLV group, spumavirus (Coffin, (1996)).

In particular the insulator element replaces at least a part of a U3 region of said retrovirus vector. Similarly the replacement can be made in another portion of the virus.

In retroviruses, the deletion of one or more essential elements such as the virus enhancer generates a disabled vector known as a self-inactivating construct (SIN) with reduced virus titer and infectious potential. Such constructs following genomic insertion can not generate further infectious virions. SIN vectors have been shown to be less prone to tumour-induction (Montini et al, 2006 & 2007) but do retain some of the oncogenic potential of unmodified vectors.

Alternatively, the retrovirus vector may be a SIN vector prior to insulator element insertion.

In particular the retroviral vector comprises an enhancer.

According to the present invention an enhancer is a DNA sequence or a fragment thereof which when placed in functional combination with a sequence encoding a gene causes an increase in the expression of the gene.

Examples of transfectable reporter or heterologous genes that can be used in the present invention include those genes whose function is desired or needed to be expressed in vivo or in vitro in a given cell or tissue type. Genes having significance for genetic or acquired disorders are particularly appropriate for use in the constructs and methods of the invention. Genes that may be insulated by the insulator elements of the present invention may be selected from, but are not limited to, both structural and nonstructural genes, or subunits thereof. Examples include genes which encode proteins and glycoproteins (e.g. factors, cytokines, lymphokines), enzymes (e.g. key enzymes in biosynthetic pathways), hormones, which perform normal physiological, biochemical, and biosynthetic functions in cells and tissues. Other useable genes are selectable antibiotic resistance genes (e.g. the neomycin phosphotransferase gene (Neo®) or the methotrexate-resistant dihydrofolate reductase (dhfr) gene) or drug resistance genes (e.g. the multi-drug resistance (MDR) genes), and the like.

Further, the genes may encode a precursor of a particular protein, or the like, which is modified intracellularly after translation to yield the molecule of interest. Further examples of genes to be used in the invention may include, but are not limited to, erythroid cell-specific genes, B-lymphocyte-specific genes, T-lymphocyte-specific genes, adenosine deaminase (ADA)-encoding genes, blood clotting factor-encoding genes, ion and transport channel-encoding genes, growth factor receptor- and hormone receptor-encoding genes, growth factor- and hormone-encoding genes, insulin-encoding genes, transcription factor-encoding genes, protooncogenes, cell cycle-regulating genes, nuclear and cytoplasmic structure-encoding genes, and enzyme-encoding genes.

Examples of eukaryotic promoters suitable for use in the invention are may include, but are not limited to, the thymidine kinase (TK) promoter, the alpha globin, beta globin, and gamma globin promoters, the human or mouse metallothionein promoter, the SV40 promoter, retroviral promoters, cytomegalovirus (CMV) promoter, and the like. The promoter normally associated with a particular structural gene which encodes the protein of interest is often desirable, but is not mandatory. Accordingly, promoters may be autologous (homologous) or heterologous. Suitable promoters may be inducible, allowing induction of the expression of a gene upon addition of the appropriate inducer, or they may be non-inducible.

Further, a variety of eukaryotic enhancer elements may be used in the constructs of the invention. Like the promoters, the enhancer elements may be autologous or heterologous. Examples of suitable enhancers include, but are not limited to, erythroid-specific enhancers, (e.g. as described by Tuan, D. et al., and in U.S. Pat. No. 5,126,260 to I. M. London et al.), the immunoglobulin enhancer, virus-specific enhancers, e.g. SV40 enhancers, or viralLTRs, pancreatic-specific enhancers, muscle-specific enhancers, fat cell-specific enhancers, liver specific enhancers, and neuron-specific enhancers.

A further object of the invention is to provide a method for detecting a DNA molecule having insulator and/or boundary properties. Said method comprises the steps of

a) providing an expression vector wherein said isolated DNA molecule as described above is positioned between a potent enhancer and a promoter domain operably linked to a reporter gene,
b) introducing the expression vector of step a) into a cell,
c) quantifying the expression of the reporter gene, and
d) correlating said reporter gene expression to potential insulator or boundary properties of said DNA molecule.

Preferably, the potent enhancer is a retroviral enhancer and in particular the reporter gene encodes for a fluorescent protein

In one embodiment of the invention, the expression vector comprises an additional gene which is not submitted to the activity of the insulator.

Any isolated DNA molecule having insulator or boundary properties identified according to said methods is also encompassed by the present invention.

The invention further provides a method and constructs to insulate the expression of a gene or genes in transgenic animals such that the transfected genes will be able to be protected and stably expressed in the tissues of the transgenic animal or its offspring, for example, even if the DNA of the construct integrates into areas of silent or active chromatin in the genomic DNA of the host animal.

By insulating a gene or genes introduced into the transgenic animal, the expression of the gene (s) will be protected from negative or inappropriate regulatory influences in the chromatin at or near the site of integration. In addition, the insulator will prevent inappropriate or unwanted activity from external enhancers that may affect the expression of the gene that has integrated into the DNA of a host cell.

The use of constructs harboring the insulator segment is envisioned for the creation of knockout mice to determine the effects of a gene on development, or for the testing of therapeutic agents, such as chemotherapeutic or other types of drugs.

Also provided is a kit or kits containing the vector constructs of the invention and used to insulate the expression of a heterologous gene or genes integrated into host DNA. The insulator element-containing plasmids or vectors may be provided in containers (e.g. sealable test tubes and the like) in the kit and are provided in the appropriate storage buffer or medium for use and for stable, long-term storage. The medium may contain stabilizers and may require dilution by the user. Further, the constructs may be provided in a freeze-dried form and may require reconstitution in the appropriate buffer or medium prior to use.

A further object of the invention is to provide a method for treating a subject diagnosed with a genetic disease, the method comprising administering the expression vector as described above so as to complement the genetic deficiency.

In a preferred aspect of the present invention, the insulator element is situated between the enhancer and the promoter of a given gene to buffer the effects of a cis-acting DNA region on the promoter of the transcription unit. In some cases, the insulator can be placed distantly from the transcription unit. In addition, the optimal location of the insulator element can be determined by routine experimentation for any particular DNA construct. The function of the insulator element is substantially independent of its orientation.

The terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment. However, in other embodiments, the subject can be a normal subject.

“Treatment” refers to both therapeutic treatment and prophylactic or preventative measures. Those in need of treatment include those already with the disorder as well as those in whith the disorder is to be prevented. Hence, the mammal to be treated herein may have been diagnosed as having the disorder or may be predisposed or susceptible to the disorder.

“Mammal” for purposes of treatment refers to any animal classified as a mammal, including humans, domestic and farm animals, and zoo, sports, or pet animals, such as dogs, horses, cats, cows, monkeys etc. Preferably, the mammal is human.

The constructs as described herein may be used in gene transfer and gene therapy methods to allow the protected expression of one or more given genes that are stably transfected into the cellular DNA. The constructs of the invention would not only insulate a transfected gene or genes from the influences of DNA surrounding the site of integration, but would also prevent the integrated constructs from impacting on the DNA at the site of integration and would therefore prevent activation of the transcription of genes that are harmful or detrimental to the cell.

When used in gene transfer and gene therapy, the constructs described herein may be administered in the form of a pharmaceutical preparation or composition containing a pharmaceutically acceptable carrier, diluent, or a physiological excipient, in which preparation the vector may be a viral vector construct, or the like, to target the cells, tissues, or organs of interest. The composition may be formed by dispersing the components in a suitable pharmaceutically-acceptable liquid or solution such as sterile physiological saline or other injectable aqueous liquids. The composition may be administered parenterally, including subcutaneous, intravenous, intramuscular, or intrasternal routes of injection. Also contemplated are intranasal, peritoneal or intradermal routes of administration. For injectable administration, the composition is in sterile solution or suspension or may be emulsified in pharmaceutically- and physiologically-acceptable aqueous or oleaginous vehicles, which may contain preservatives, stabilizers, and material for rendering the solution or suspension isotonic with body fluids (i.e. blood) of the recipient. Excipients suitable for use are water, phosphate buffered saline, pH 7.4, 0.15 M aqueous sodium chloride solution, dextrose, glycerol, dilute ethanol, and the like, and mixtures thereof. The amounts or quantities, as well as routes of administration, used are determined on an individual basis, and correspond to the amounts used in similar types of applications or indications known to those of skill in the art.

A still further object is to provide a host cell comprising the isolated DNA molecule of the invention and/or at least one copy of the expression vector of the invention as described herein.

Preferably, the host cell consists of a stem cell, a cultured cell or an ex vivo transduced cell. Many types of cells and cell lines (e.g. primary cell lines or established cell lines) and tissues are capable of being stably transfected by or receiving the constructs of the invention. Examples of cells that may be used include, but are not limited to, stem cells, B lymphocytes, T lymphocytes, macrophages, other white blood lymphocytes (e.g. myelocytes, macrophages, monocytes), immune system cells of different developmental stages, erythroid lineage cells, pancreatic cells, lung cells, muscle cells, liver cells, fat cells, neuronal cells, glial cells, other brain cells, transformed cells of various cell lineages corresponding to normal cell counterparts (e.g. K562, HEL, HL60, and MEL cells), and established or otherwise transformed cells lines derived from all of the foregoing. In addition, the isolated DNA molecule of the present invention may be transferred by various means directly into tissues, where they would stably integrate into the cells comprising the tissues. Further, the vectors containing the insulator elements of the invention can be introduced into primary cells at various stages of development, including the embryonic and fetal stages, so as to effect gene therapy at early stages of development.

Another aspect of the invention is the use of the isolated DNA molecules as described herein as insulator or boundary sequences.

Finally, the invention also provides a mammalian cell stably transfected with the isolated DNA molecule and/or at least one copy of the expression vector of the invention.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1

### Materials and Methods

### 1.1 Plasmid Vectors

The minimal CMV promoter and EGFP and DsRed coding sequences (Clontech) were PCR amplified and cloned in both orientations in pBS-SK2 containing telomeric repeats, kindly provided by J. Baur (2). Puromycin resistance gene expressed from the CAG promoter was inserted upstream of DsRed, in a telomere-distal position. Four Gal4 binding site were introduced between EGFP and DsRed expression cassettes at AscI and BamHI restriction sites, yielding pGE1min-Gal and pGE2 min-Gal. Control plasmids were generated by deletion of the telomeric repeats. Plasmids encoding the Gal DNA binding domain alone (pCD-Gal-DBD), or fused to the CTF1 Proline rich (pCMV-Gal-Pro) or to the VP16 (pCMV-Gal-VP16) transcriptional activation domains were as described previously (31). Plasmids encoding Gal-CTF1 fusion mutations were previously described by Alevizopoulos et al. (1995). Plasmids used to generate stable populations expressing Gal4 derivatives were obtained by cloning Gal-fusion genes or the BFP gene in an expression vector carrying the MAR 1-68 and the SV40 promoter (15).

### 1.2 Cells Culture, Transfection and In Situ Hybridization

HeLa cells (Clontech) were cultivated at 37° C. and 5% CO2 in DMEM-F12 with 10% fetal bovine serum (Gibco). Histone deacetylation or DNA methylation studies were performed by supplementing the cell culture medium with either 1 μM of Trichostatin A (TSA, Wako) for 48 h, 1 mM of Sodium butyrate (Sigma) for one week, 3 μM of 5-aza-2′-deoxycytidine (5azadC, Sigma) for 48 h, or 50 μM of Bromo-deoxyuridine (BrdU, Applichem) for one week. Transfections were performed using the Fugene 6 transfection reagent following instructions from the manufacturer (Roche). Stable clones were obtained by transfection of linearized plasmids pGEmin-Gal, pGE2 min-Gal or their respective controls. Cells were selected with 2 μg/mL of puromycin for three weeks, and all analyses were performed at least two weeks after the end of selection, to allow for the silencing of the telomeric locus. Transient transfections were performed by co-transfection of a Gal-fusion encoding plasmid and a BFP encoding plasmid at a molar ratio of 9:1. Cytofluorometric assays of the fluorescent reporter proteins were performed 48 h later. Stable populations expressing Gal-DBD/Gal-Pro were obtained by co-transfecting the Gal-fusion expression plasmid, a BFP-encoding plasmid, and a Zeocin resistance plasmid at 45:45:10 weight ratio. Zeocin resistant cells displaying high BFP levels were sorted twice, and the amount of zeocin was increased to 1800 μg/mL with increments of 200 μg/mL, to ensure consistent and elevated levels of fusion protein expression. Fluorescence in situ hybridization (FISH) was performed as described previously (12, 15) using two colors labeling of the reporter plasmids and of the telomeric repeats.

### 1.3 Chromatin Immuno-Precipitation

Antibodies against acetylated H3 (06-599), acetylated H4 (06-866) and trimethylated H3K9 (07-442) were obtained from Upstate biotechnology. Antibody against H2A.Z (ab4174) was purchased from Abcam. HeLa cells were harvested at a confluence of 90% and cross-linked with 1% formaldehyde for 4 min. After lysis of the nuclei, chromatin was sonicated to obtain fragments of ˜1000 pb and digested with BamHI. The chromatin solution was diluted to a volume of 300 μL in a buffer containing 200 mM HEPES, 2M NaCl, 20 mM EDTA, 0.1% NaDoc, 1% Triton X-100, 1 mg/mL BSA. Chromatin fragments were precleared 30 min with 10 μL rProtein A Sepharose (Amersham Biosciences) and supernatants were incubated at 4° C. overnight with 5 μL of antibody. Immunoprecipitated complexes were incubated with 10 μL rProtein A Sepharose and pellets were washed 3 times with IP buffer (20 mM HEPES, 0.2M NaCl, 2 mM EDTA, 0.1% NaDoc, 1% Triton X-100). Immunoprecipitated complexes were eluted in 100 mM Tris/HCl, 1% SDS and cross-links were reversed at 65° C. for 1 hour. Precipitated DNAs were eluted in 50 μL TE.

### 1.4 Quantitative PCR

Quantitative PCR was performed on 7700 Sequence detector (Applied Biosystems) using SYBR Green reagent (Eurogentec). Chromatin immuno-precipitation samples and chromatin input were diluted 10 fold before analysis. GAPDH amplification was performed using 5′-CGCCCCCGGTTTCTATAA-3′ (SEQ ID No 4) and 5′-ACTGTCGAACAGGAGGAGCAG-3′ primers, EGFP using 5′-AGCAAAGACCCCACCGAGAA-3′ and 5′GGCGGCGGTCACGAA-3′ primers and DsRed using 5′-TTCCAGTACGGGTCCAAGGT-3′ and 5′-GGAAGGACAGCTTCTTGTAGTCG-3′ primers. EGFP and DsRed values were normalized by GAPDH.

### Example 2

### Results

### 2.1 Design of a Telomeric Gene Silencing Quantitative Assay

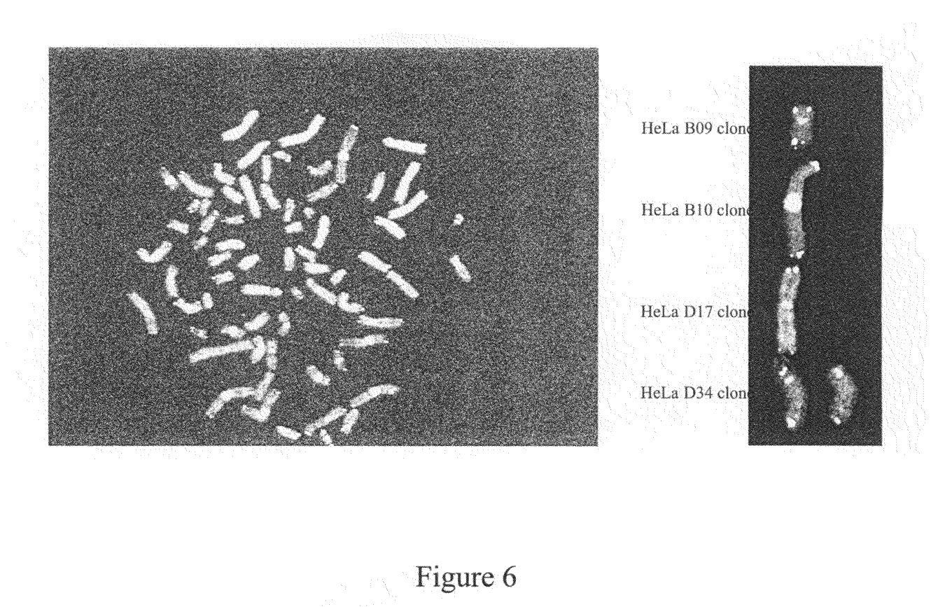

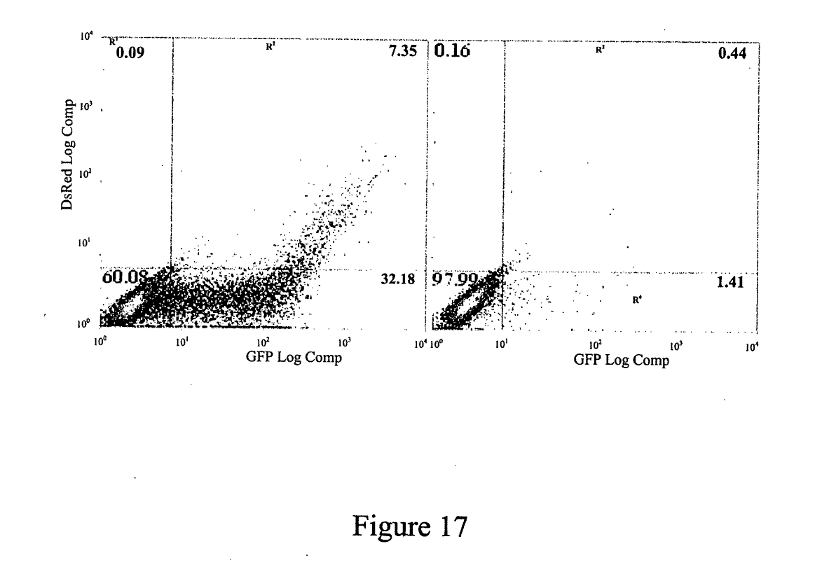

In order to analyze both telomere-insulated and non-insulated genes co-integrated at the same telomeric locus, we generated the reporter plasmids shown in FIG. 17A. Reporter vectors consist of the green fluorescent protein (GFP) and red fluorescent protein (DsRed) coding sequences placed on either side of four DNA binding sites for the yeast GAL4 protein. Each reporter gene was placed under the control of a minimal CMV promoter, in an orientation mediating either convergent or divergent directions of transcription. An antibiotic resistance gene was placed adjacent to DsRed, while telomeric (TTAGGG)n repeats were placed next to the GFP expression cassette. Previous studies have demonstrated that stable transfections of telomeric repeat-containing plasmids yield mostly single copy integration at a telomeric position, possibly because integration of the telomeric repeats induces a chromosomal break and the formation of a new telomere (2, 32, 35). These constructs were transfected, and antibiotic-resistant cells having stably integrated the transgenes in their genome were selected and sorted into monoclonal populations. Clones showing the following properties were discarded: (1) heterogeneous or disproportionate DsRed and GFP fluorescence, probably because of multiple insertions and/or a non-clonal nature, (2) no activation of DsRed and/or GFP upon transfection of a Gal-VP16 expression vector, which suggests the deletion of one or both reporter genes, and (3) high basal expression of GFP and DsRed, which may result from non-telomeric integrations. Fluorescence in situ hybridization (FISH) analysis indicated a telomeric or subtelomeric transgene position for all retained clones (see FIG. 6 in the supplemental material). Monoclonal populations were also generated from the transfection of reporter plamids deleted of the telomeric repeats, to obtain integration at non-telomeric loci, and cell clones were selected similarly according to the above criteria 1 and 2.

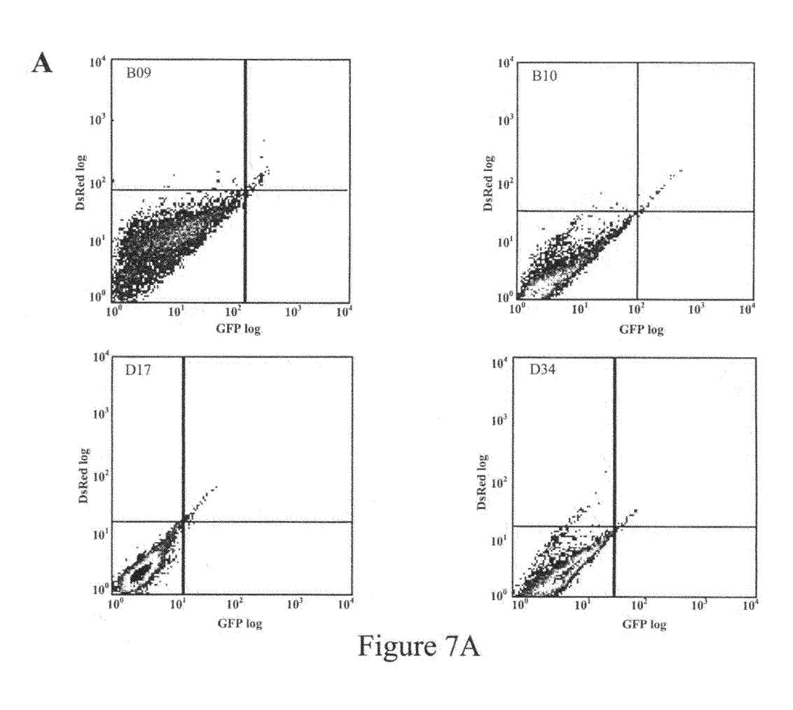

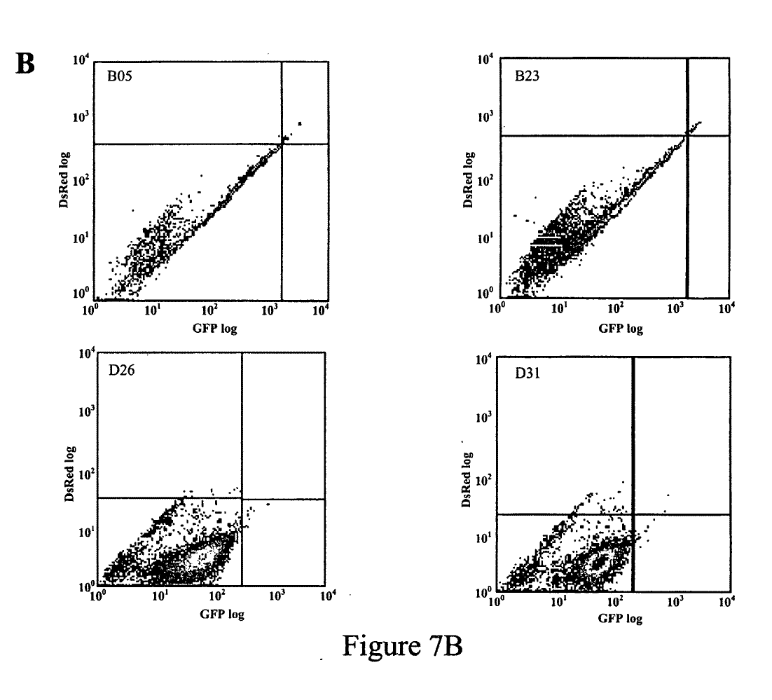

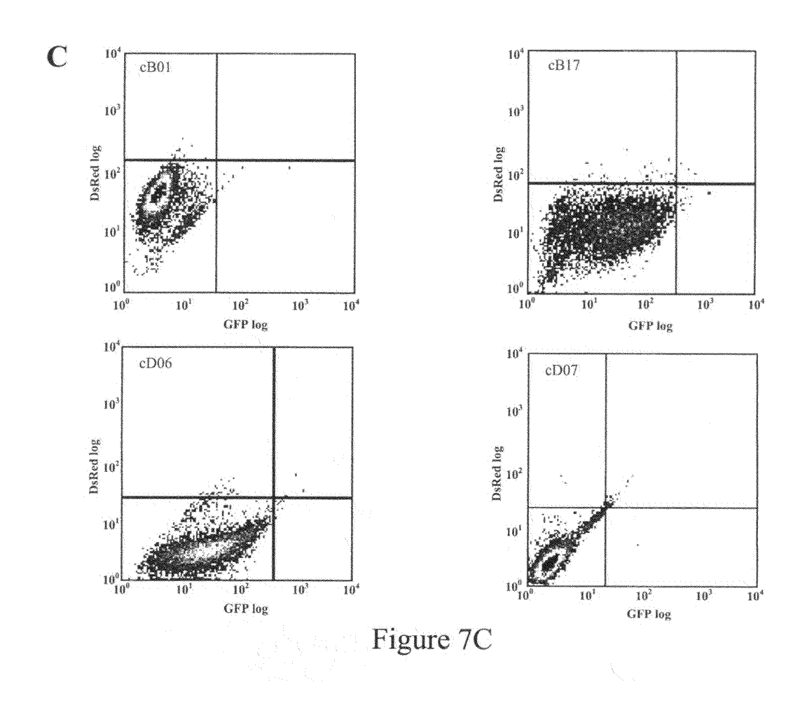

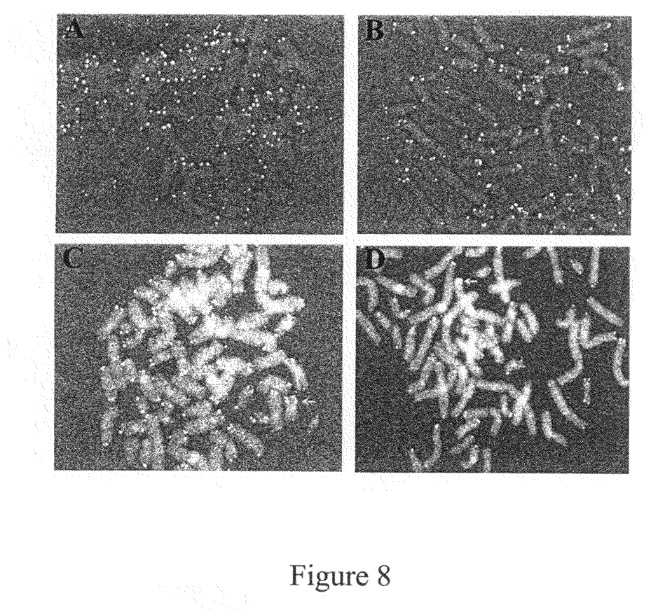

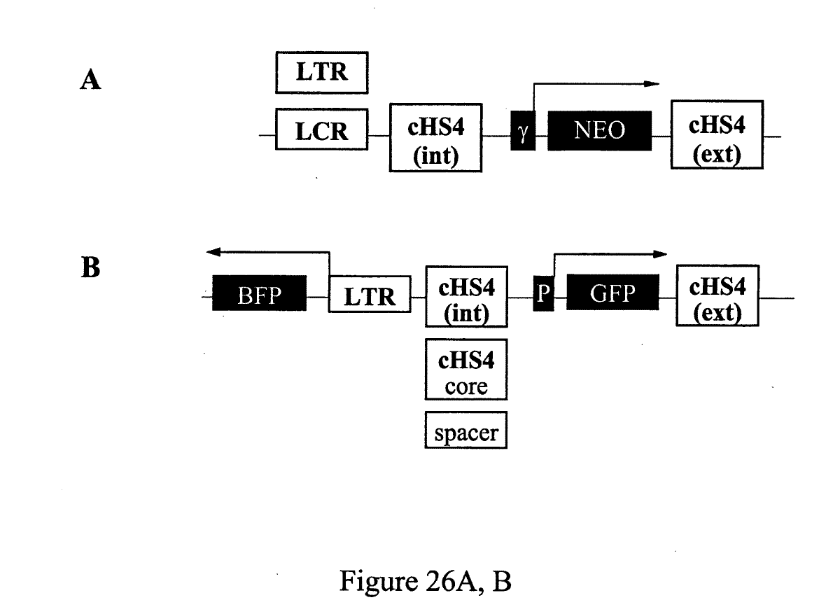

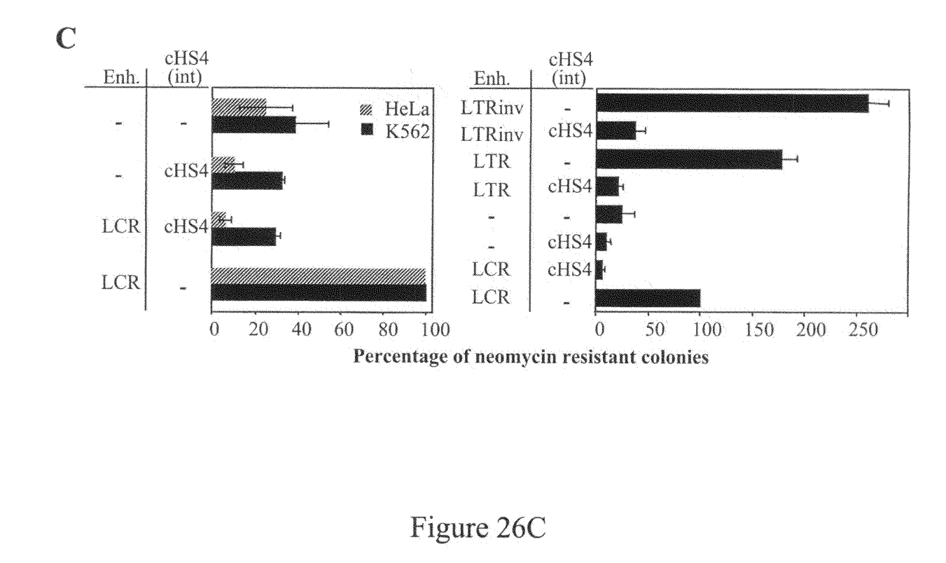

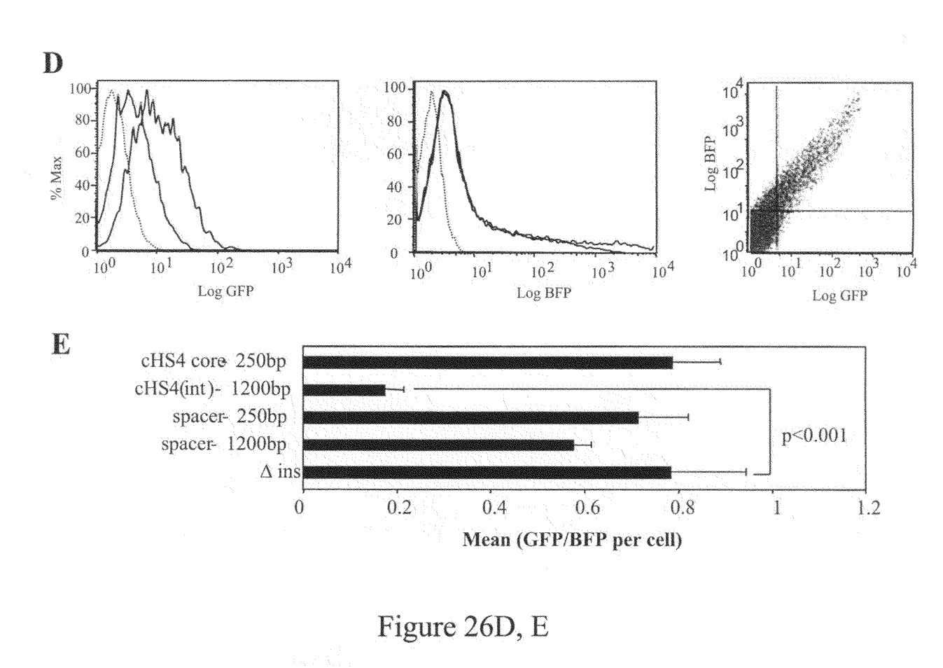

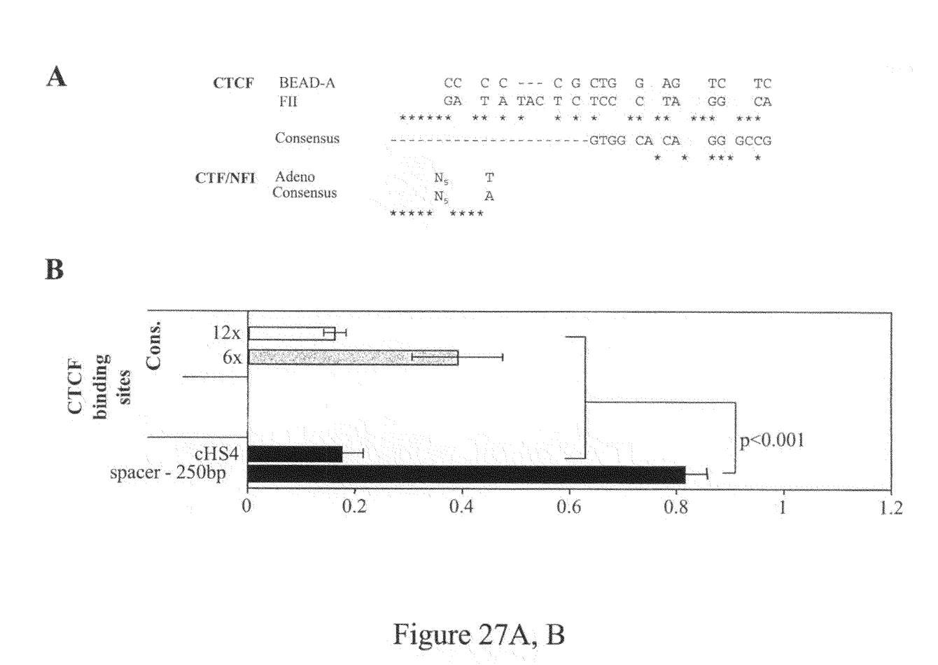

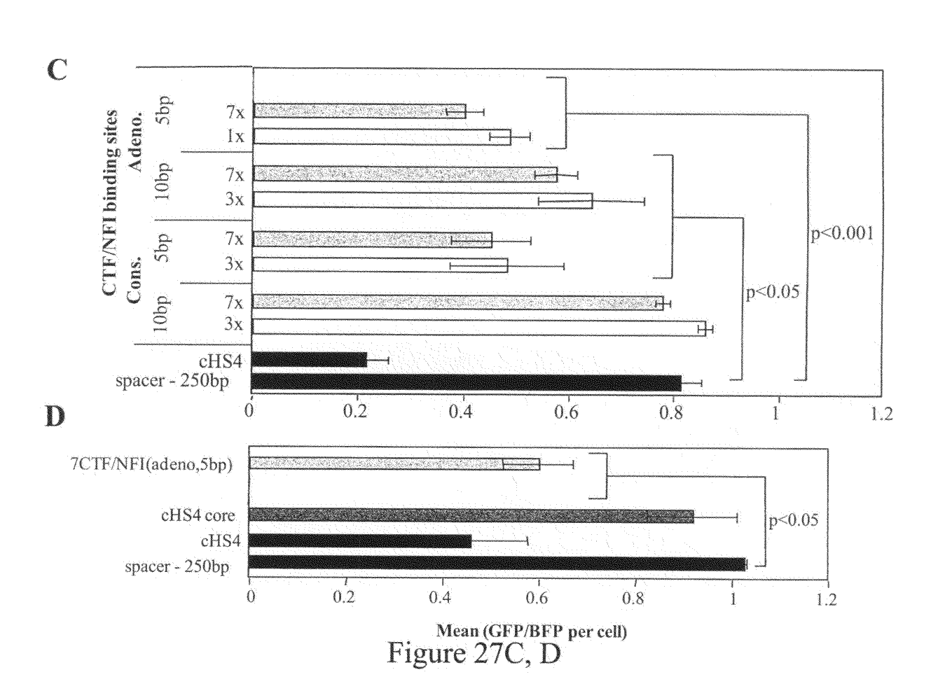

This yielded three categories of clonal populations. The first two categories, generated from telomeric repeat-containing plasmids, display a telomeric or subtelomeric transgene location and nearly undetectable reporter gene expression, or low but detectable transgene expression (see FIGS. 26 and 27). These results are consistent with previous reports of the low expression of telomeric transgenes in mammalian cells (2, 32, 35). The last category of clones generated using constructs devoid of telomeric repeats, displayed random chromosomal integration sites and variable levels of expression (see FIGS. 7 and 8). Clones displaying clear internal chromosome integration and relatively low expression levels were kept as controls.

### 2.2 CTF1 Protects Telomeric Transgenes from TPE

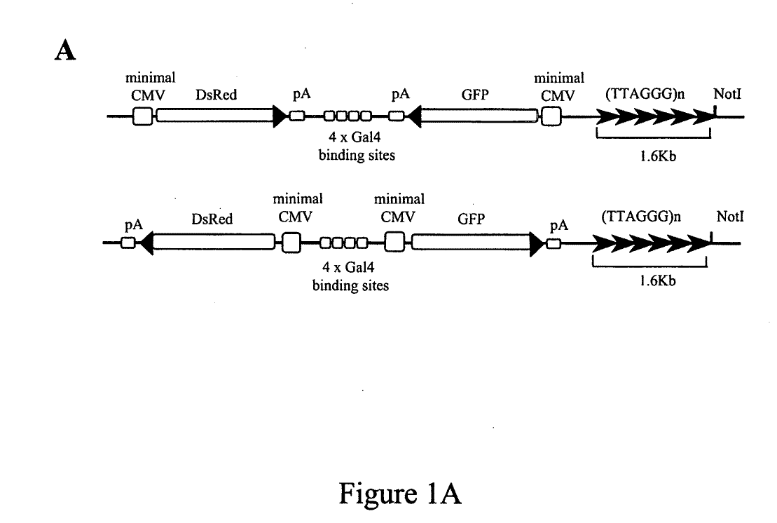

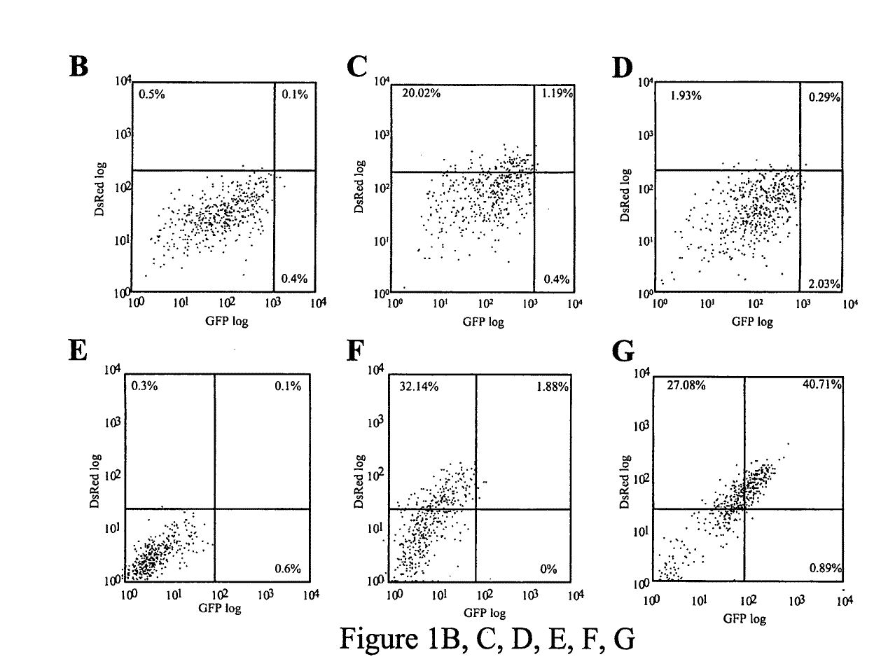

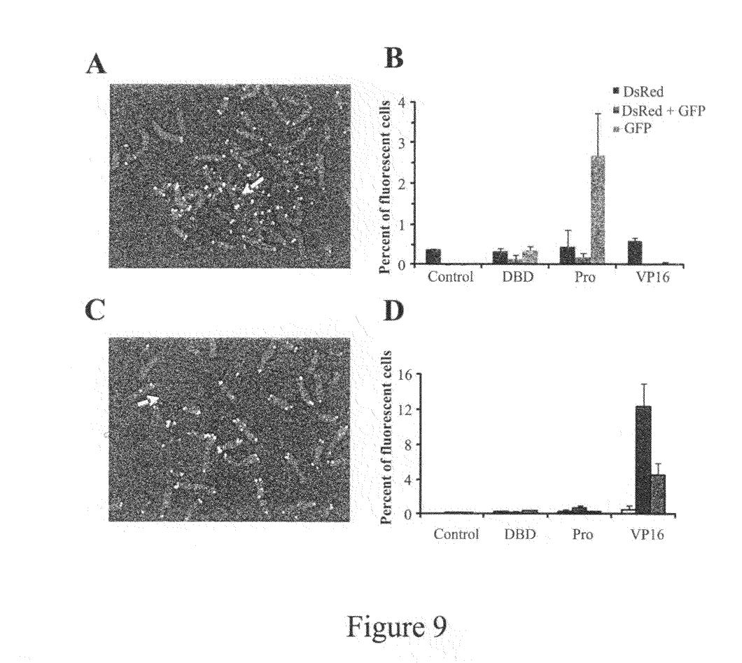

The proline-rich domain of CTF1 has been shown to interact with histone H3.3 and to activate gene transcription in response to growth factors in mammalian cells (1). To specifically assess CTF-1 activity at mammalian telomeres, and to exclude possible interference from other members of the HeLa cell CTF/NF1 family (36), the CTF1 proline-rich domain was transiently expressed as a fusion to the DNA binding domain of the yeast GALA protein (Gal-Pro). Expression vectors encoding either the unfused GALA DNA-binding domain (Gal-DBD), or a fusion with the strong herpes simplex virus VP16 activator (Gal-VP16), were used as controls. These plasmids were co-transfected with a blue fluorescent protein (BFP) expression vector as a transfection marker, and transiently transfected BFP expressing cells were analyzed for GFP and DsRed fluorescence. Gal-Pro expression resulted in an increase of DsRed fluorescence without an increase of GFP fluorescence in the telomeric clones (compare FIGS. 1B and 1E with 1C and 1F, respectively). Moreover, the use of derivative plasmid constructs carrying the DsRed and GFP in a reversed configuration confirmed that the expression of the telomere distal gene in clones expressing Gal-Pro is not gene specific, but is dependant on its position in relation to the telomere and to the GAL-Pro binding sites (FIG. 9). In contrast, the Gal-VP16 fusion did not significantly activate the transgenes, when transcribed in a convergent fashion (FIG. 1B, 1D, and FIG. 9B), while it activated DsRed and GFP divergent transcription to a similar extent (FIG. 1E, 1G and FIG. 9D). The lower activation of the convergent construct is explained by the more distant location of the Gal-VP16 binding sites from the promoters driving the reporter genes (FIG. 1A, top drawing). Assays of GAL4 fusions to other proteins that bind insulator and/or boundary elements, such as CTCF or USF1 (2, 32, 35), failed to affect DsRed or GFP expression (data not shown), confirming a specific function of CTF1 at the telomeric loci.

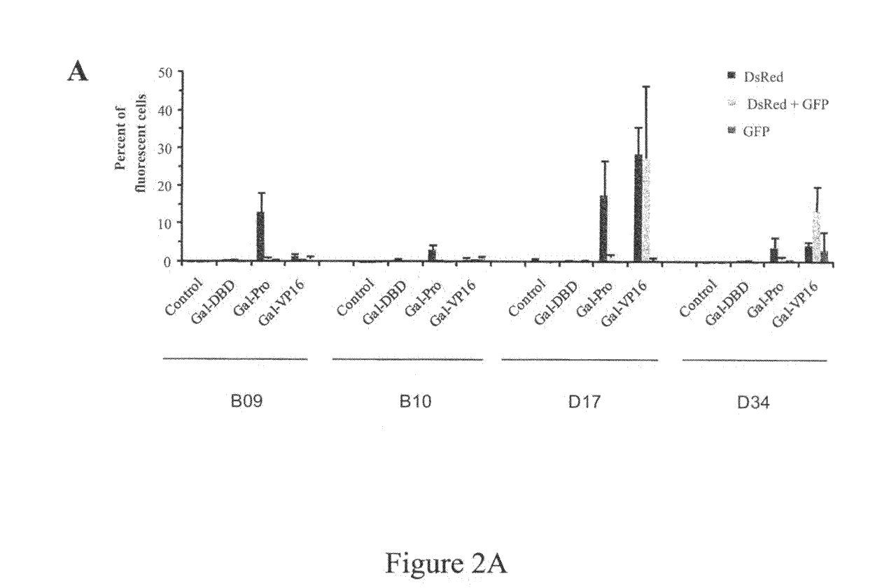

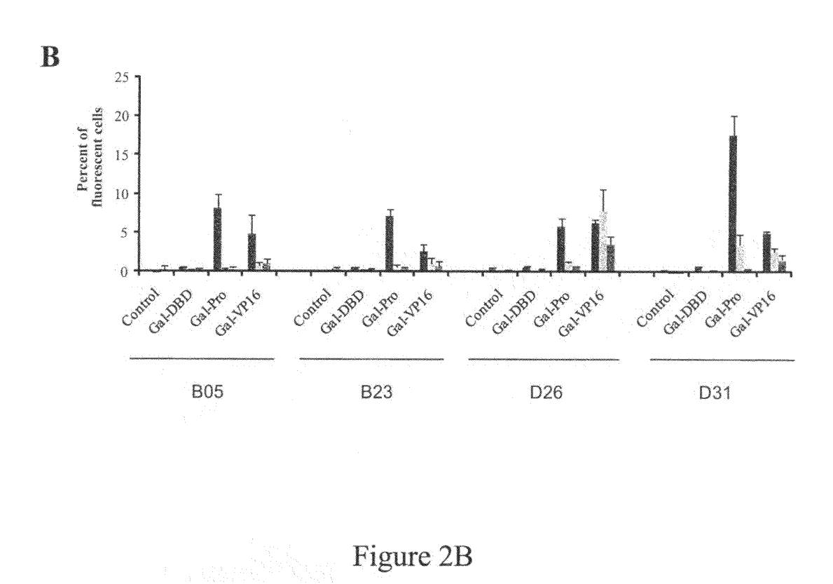

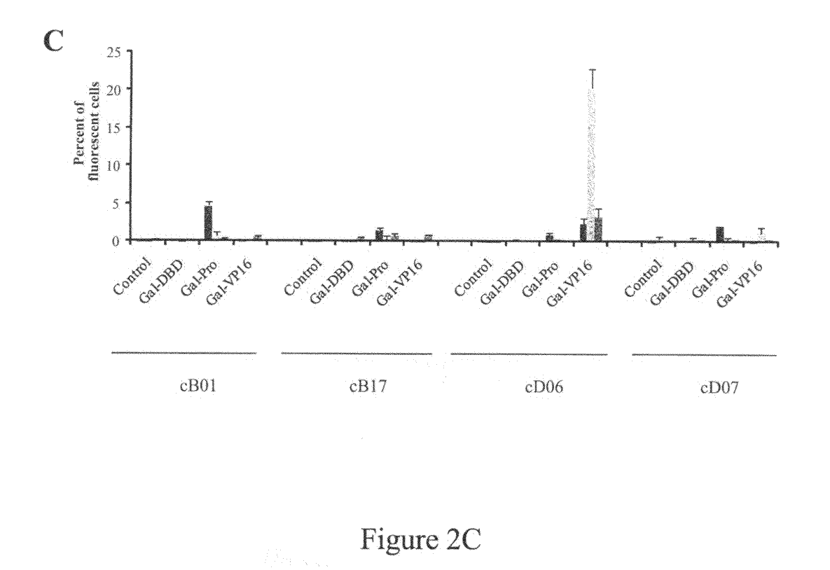

Quantification of the Gal-Pro effect indicated that it occurs in independent clones that have a telomeric transgenes integrated in various chromosomes (FIGS. 2A and 2B, and FIG. 7). In contrast, Gal-VP16 activated the expression of the reporter genes to a variable extent, but without a marked preference for the activation of DsRed over GFP. Gal-Pro had variable but generally smaller effects on the expression of transgenes integrated at non-telomeric positions, where it could also activate GFP expression (FIG. 2C).

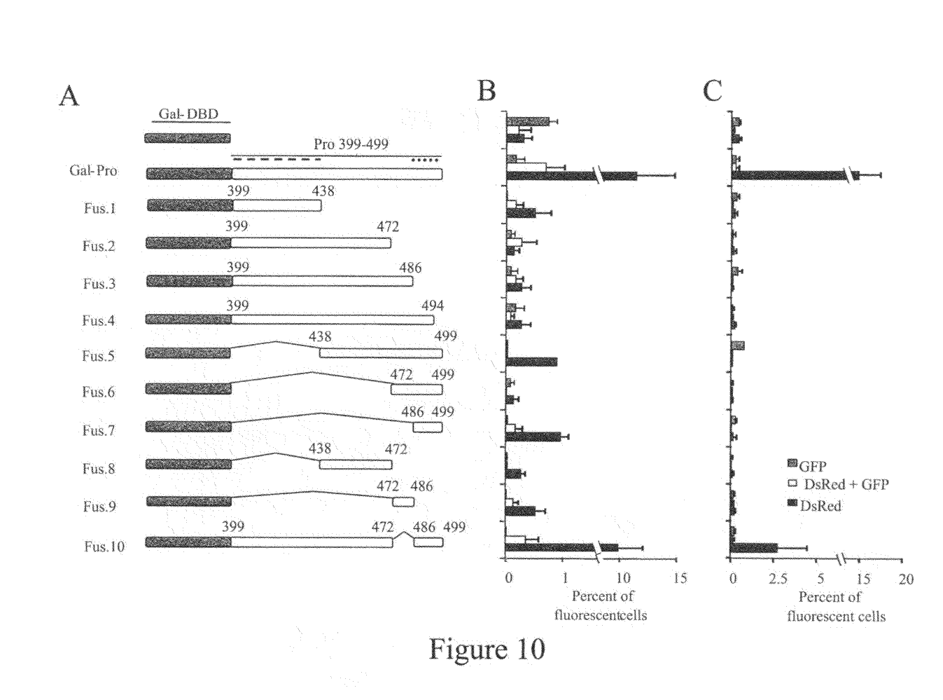

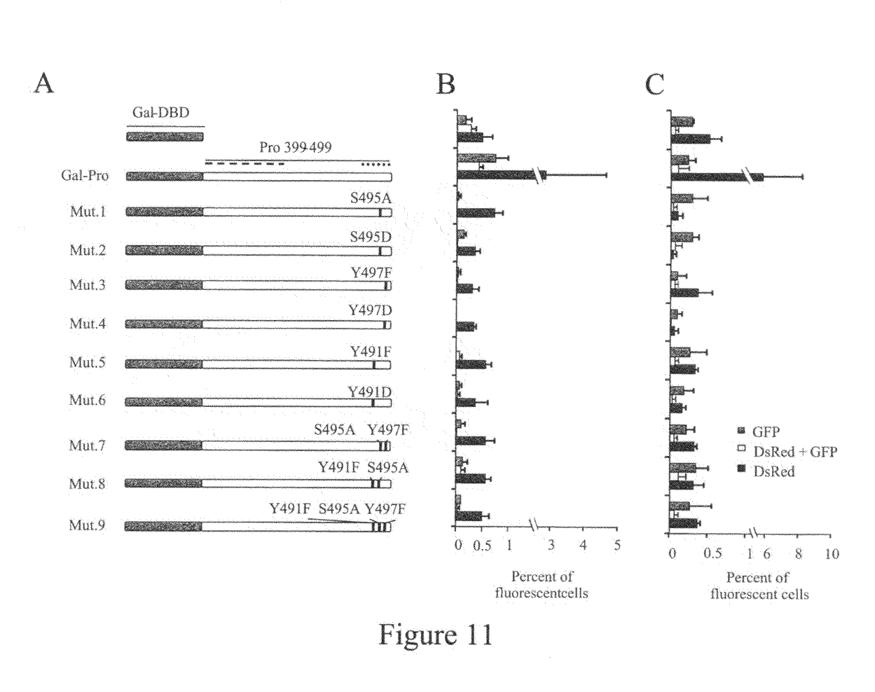

We previously showed that the proline-rich activation domain of CTF1 possesses two regions that cooperate to bind histone H3, and that this domain may reposition nucleosomes close to its binding site (11, 30, 31). Thus, we assessed whether the H3 interaction domains may mediate the boundary activity. Gal-fusions previously characterized by their ability to bind H3 were expressed in telomeric clones B09 and D17, where Gal-Pro shows strong boundary effects. In both cases, deletions in the H3 interaction domains were associated with a reduction of DsRed expression (see FIG. 10), matching precisely the results on interaction with histone H3.3 as probed by two-hybrid assays Alevizopoulos et al. (1995). Similarly, single proline rich domain point mutations known to decrease or abolish interaction with H3 reduced consistently the boundary effect (see FIG. 11). Taken together, these data are consistent with a role of the H3 interaction in the boundary activity.

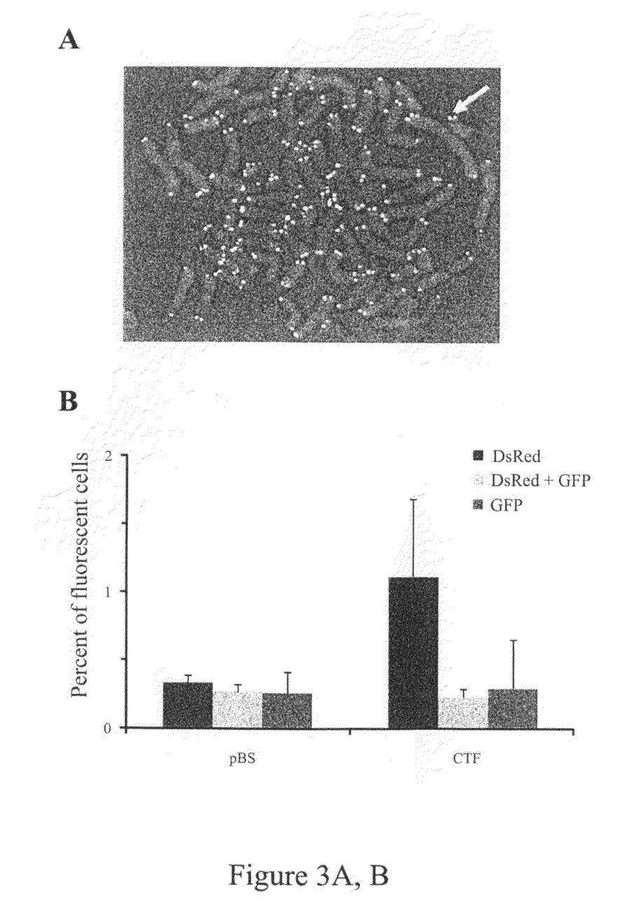

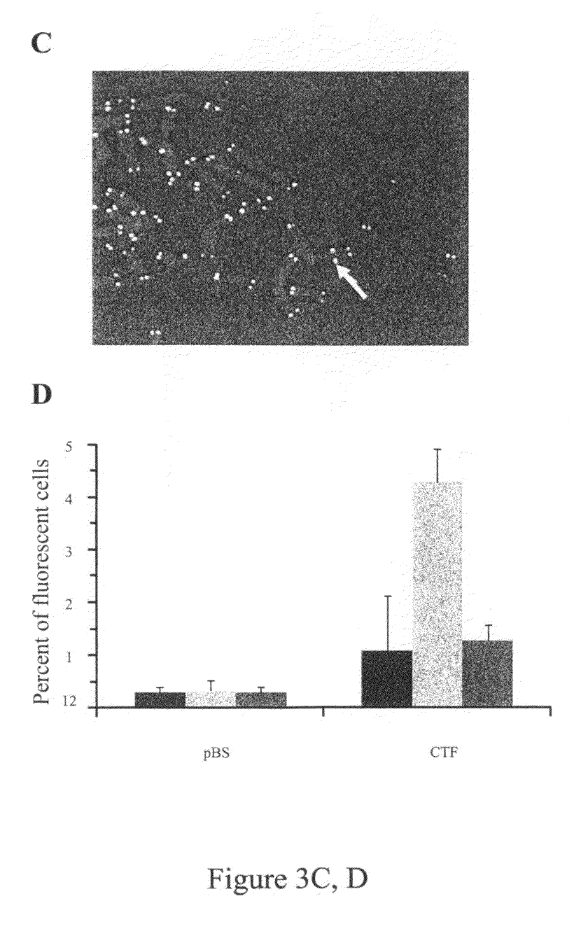

To assess if the boundary effect can also be observed from the expression of native transcription factors such as CTF1, we analyzed clones generated with reporter constructs carrying seven CTF/NF1 binding sites inserted between the two reporter genes instead of the GAL4 sites. Since various members of the family of CTF/NF1 proteins are expressed in HeLa cells (36), we sought to identify clones in which the additional expression of CTF1 may mediate a boundary effect. Clones having integrated the reporter genes in telomeric or internal chromosomal positions were thus isolated and analyzed after the transient expression of CTF1. The boundary effect was observed upon CTF1 expression in cells with telomeric transgenes (FIGS. 3A and 3B), while a commensurate activation occurred for both reporter genes inserted at an internal location on the chromosome (FIGS. 3C and 3D). The boundary effect at telomeric loci was observed in three independent clones with telomeric transgenes, but the boundary effect observed upon CTF1 over-expression was overall smaller that that obtained with the GAL4 fusion protein (data not shown). This may stem from the background of CTF/NF1 proteins, as they may already mediate some boundary effects on the reporter constructs containing CTF/NF1 binding sites, and/or from the stronger interaction of GAL4 to heterochromatic DNA as compared to CTF1 (31).

Taken together, these results indicate that CTF-1 and its fusion derivatives act specifically to prevent silencing of the telomere distal but not of the telomere-proximal gene, implying that they may prevent the propagation of a silencing signal from the telomere towards more centromeric sequences. Thus, these results suggested that this protein may act as a boundary or barrier element that blocks the spreading of a repressive chromatin structure from the telomere.

### 2.3 Chromatin Landscape at Mammalian Telomeric Loci

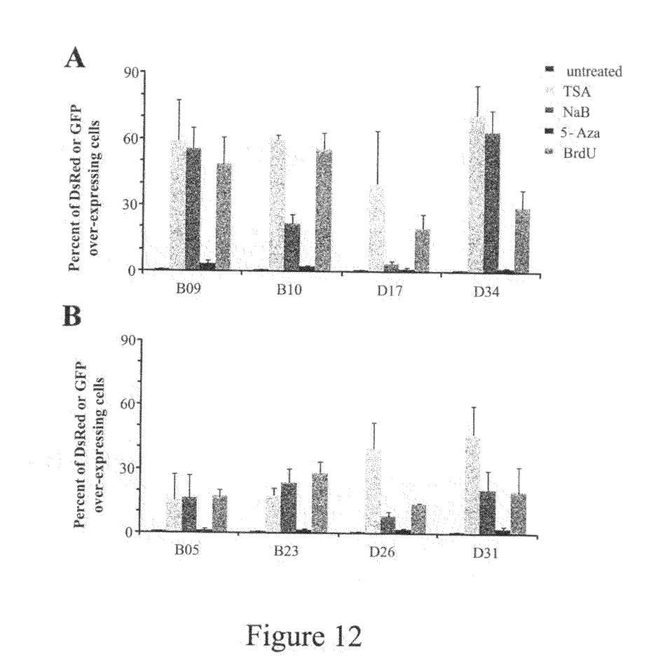

Chemical agents that affect histone acetylation or DNA methylation were used to assess whether telomeric transgenes are subjected to chromatin-mediated silencing effects. Trichostatine A (TSA), a broad-specificity inhibitor of class I and II histone deacetylase (HDAC) was found to strongly increase transgene expression at various telomeric positions in independent cell lines (see FIG. 12). In contrast, sodium butyrate (NaB), a more specific inhibitor of HDAC I and IIa classes, mediated lower unsilencing effects in some clones, suggesting an involvement of the HDAC IIb class in gene silencing at some but not all telomeres. Thus, several HDAC activities may be involved in telomeric gene silencing. HDAC inhibitor treatment of telomeric clones with lower transgene expression generally resulted in greater enhancement of gene expression, as would be expected from a chromatin-mediated silencing process (compare FIGS. 6A and 6B with 12A and 12B).

Treatment of telomeric clones with the 5-aza-2′-deoxycytidine (5azadC) DNA-methylation inhibitor had little effect on transgene expression (FIG. 11). Thus, DNA methylation is unlikely to be the primary determinant of telomeric silencing in this cellular model. Several studies have shown that Bromodeoxyuridine (BrdU) can abolish expression variegation, namely the cycling between semi-stable expressing and non-expressing states. Its mode of action remains unclear, but it may act by decreasing histones mobility (25). BrdU treatment of telomeric clones was associated with an increase in expression of the reporter genes, but to a lesser extent than that noted with TSA, suggesting that telomeric silencing involves chromatin remodeling.

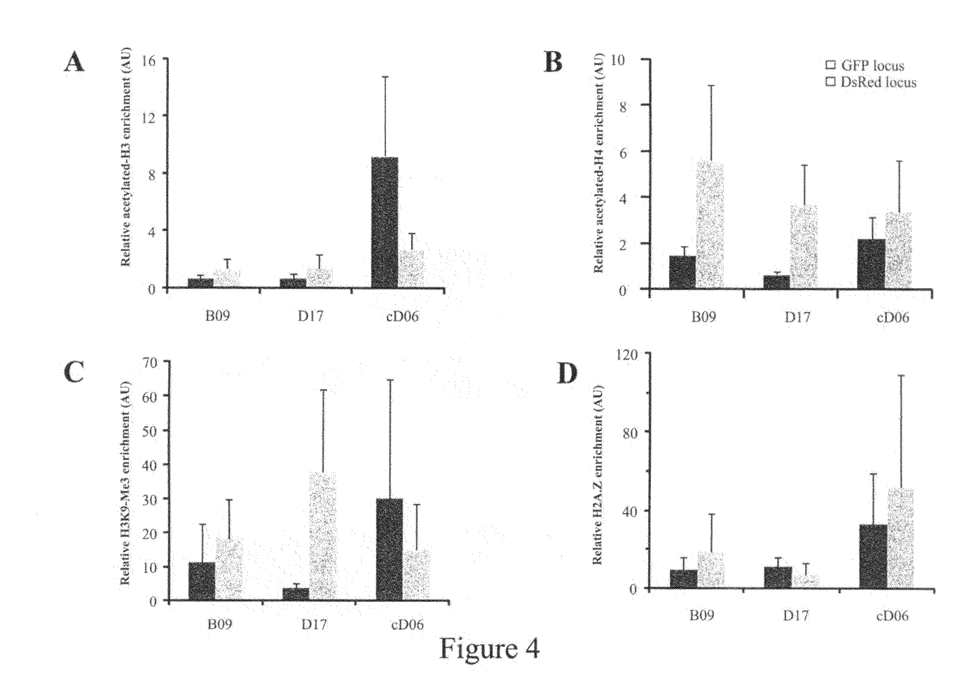

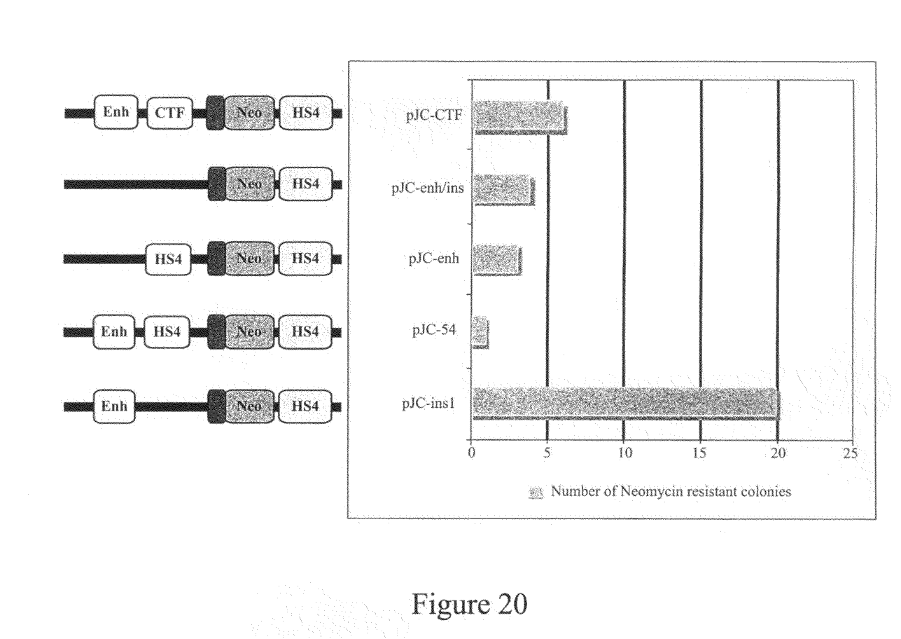

The involvement of nucleosome hypoacetylation in the silencing of telomeric genes was further analyzed by chromatin immunoprecipitation assays (ChIP) of two clonal populations showing strong telomeric silencing. This revealed hypoacetylation of H3 over both the GFP and DsRed telomeric sequences, but the effect was more prominent on the telomere-proximal GFP gene, as compared to the telomere distal DsRed sequence. This does not stem from preferential acetylation of the latter gene, as high levels of acetylated H3 were found on both transgenes integrated at an internal locus in the cD06 cells (FIGS. 4A and 4B). Hypoacetylation of histone H4 was only observed on the GFP sequence, further arguing for a correlation between telomere proximity and the histone hypoacetylation effect (FIG. 20B). This finding is consistent with the spread of a silencing signal from the telomeric repeats, and it is reminiscent of distance-related silencing effects associated with the propagation of a silent chromatin structure from yeast telomeres (23).

The trimethylation of histone H3 on lysine 9 (H3K9Me3) has been associated with heterochromatin-mediated gene silencing (4, 34, 37). However, H3K9Me3 levels were not significantly elevated in the telomeric clones as compared to the transgenes integrated at an internal position (FIG. 4C). Rather, low H3K9Me3 modifications on clone D17 GFP sequence correlates well with the low GFP expression, in contrast to clones B09 and cD06 which show moderate or high levels of both methylation and GFP expression, respectively (compare FIG. 4C and FIG. 6). Other histone modifications such as H4K20Me3, H3K27Me3 or H3K79Me2 did not have a preferred location on the telomeric genes (data not shown). The histone variant H2A.Z has often been located at the boundaries of silent and permissive chromatin domains (9, 27). Its low levels at the telomeric reporter genes of clones B09 and D17 indicate that it may be excluded from telomeric loci (FIG. 4D). Overall, these results link telomeric gene silencing to histone H3 hypoacetylation and H3K9 methylation, and they imply that a short-ranging gradient of such modifications stems from the telomere.

### 2.4 CTF1 Fusion Protein Delimits Distinct Chromatin Domains at Telomeric Boundaries

Given our conclusion that telomeric transgene silencing involves histone modifications, we next assessed if Gal-Pro expression may selectively oppose these changes over the DsRed-coding sequence. Clone B09 was stably transfected with Gal-DBD or Gal-Pro expression vectors to ensure stable expression of the GAL4 fusion in a significant proportion of the cell population. Expression of these GAL4 fusions was assessed indirectly, by measuring the fluorescence of the blue fluorescent protein (BFP) expressed from a co-transfected vector.

Gal-pro expression was associated with an increase of H3 and especially H4 acetylation on the DsRed sequence of clone B09. However, Gal-Pro expression did not affect histone acetylation on the GFP sequence, indicating that Gal-Pro mediates the formation of two chromatin domains of distinct acetylation status, but that it does not act by recruiting HATs that would acetylate bidirectionally the GFP and DsRed genes. Gal-Pro expression also strongly increased H3K9Me3 on DsRed but not GFP at the B09 telomere. The trimethylation of H31(27 and H4K20, which are modifications generally associated with gene silencing, were similarly increased on the expressed DsRed sequence in the presence of Gal-Pro (data not shown). The HDAC inhibitor TSA yielded an increase of the acetylation of both DsRed and GFP, as well as the trimethylation of H3K9, indicating that the latter modification may be a consequence of the increase in histone acetylation.

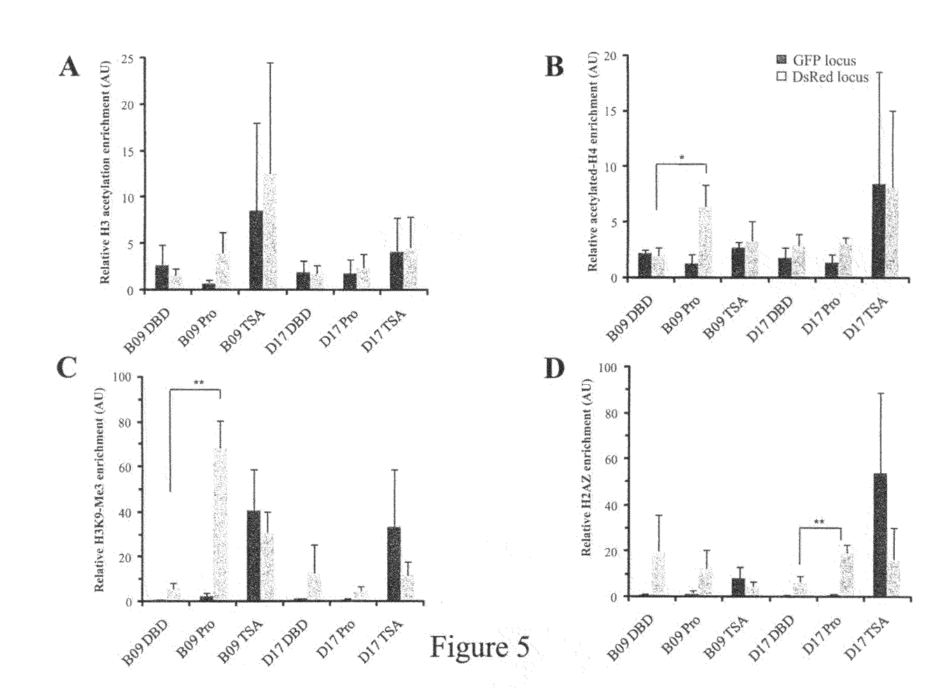

To determine if histone acetylation changes are always involved in the boundary effect, clone D17 was similarly tested, as GAL-Pro has strong boundary activity while the HDAC inhibitor NaB has little effect on telomeric gene expression (FIG. 2A and FIG. 12). Expression of Gal-Pro was not associated with an increase in H3 and H4 acetylation, nor with modifications such as H3K9Me3, H3K27Me3 or H4K20Me3 (FIG. 5 and data not shown). However, the occurrence of H2A.Z on DsRed was significantly increased. This indicates that several types of chromatin structures may be associated with telomeric silencing and insulation effects, and that Gal-Pro may act to separate chromosomal domains of distinct chromatin structures.

### Example 3

### Discussion

The eukaryotic genome is thought to be partitioned in euchromatic or heterochromatic domains in which chromatin may be either permissive for gene expression or rather silent. How the boundaries separating these chromatin domains are established, and how they may influence gene expression, remains poorly understood. In this work, we show that two genes co-localized at a telomeric locus can be partitioned into active and inactive chromatin structures by the CTF1 protein or fusions derived thereof. This mode of action is distinct from that of the VP16 transcriptional activator, which induces bi-directionally the expression of telomere proximal as well as telomere distal genes, but only over a short distance. This latter effect most likely results from the ability of VP16 to recruit HAT and components of the basal transcription machinery to the promoter (19). In contrast, CTF1 derivatives protect the telomere-distal gene from silencing effects without significantly affecting the expression of the telomere proximal gene, and irrespective of the gene orientation or distance to the promoter. This implies that CTF1 does not act as a classical transcriptional activator, but rather that it mediates the establishment of a barrier that blocks the propagation of a silent chromatin structure from the telomere, thereby forming a boundary between expressed and silent genes. The CTF-1 boundary effect is mediated by its histone-binding domain, and mutations that inhibit interactions with the histone also inhibit the boundary effect. Taken together with previous observations that CTF1 binds preferentially to the H3.3 and that this histone variant is enriched at chromatin boundaries (11, 29), these findings imply a mechanism whereby the interaction of CTF1 with nucleosomes may establish a chromosomal structure that blocks the auto-propagation of silencing signals from the telomere. These findings provide a mechanistic explanation for the previous observations that CTF1 may contribute to reversing chromatin-mediated gene silencing, but that alone it is unable to activate transcription (31).

In budding yeast, TPE is mediated by the spreading of the SIR protein complex from the telomere over subtelomeric regions, which results in histone deacetylation and gene silencing. However, a similar mechanism involving the propagation of SIR proteins has not been reported in mammalian cells. Rather, the establishment of a repressive telomeric structure has been associated with increased H3K9Me3 modifications at telomeres (4, 33). H3K9Me3 is known to bind HP1, which may in turn recruit the Suv39 HMTase to mediate further H3K9 methylation. Here, we find that histone deacetylation is linked to silencing at several of the analyzed telomeric loci, and that broad-range HDAC inhibitors such as TSA mediate not only an increase of histone acetylation, but also other types, of modification such as H3K9 trimethylation. This implies a causal effect of hypoacetylation on histone methylation levels and silencing effects in mammalian cells. This conclusion is further supported by the previous demonstration that H3K9Me3 modifications may occur as a result of gene transcription (39) and by the occurrence of H3K9Me3 on a transgene protected from a chicken telomere by the cHS4 beta-globin insulator (32, 35).

Although we observed variable degrees of histone hypoacetylation when comparing different telomeric integration loci, the extent of histone deacetylation was found to be associated with telomere proximity, as it is significantly lower over the telomeric-distal gene. This finding suggests a short-ranging spread of a hypoacetylation signal from the telomere. This contrasts with the long-ranging histone hypoacetylation and silencing that stem from yeast telomeres, and it may explain why telomeric gene silencing has been more difficult to detect in mammalian cells. In human cells, we find that expression of Gal-Pro results in the recovery of histone acetylation on the telomere-distal but not on the proximal gene, further supporting the notion that it acts to block the self-propagation of a deacetylated histone structure. This interpretation is consistent with the recent implication of the mammalian SIRT6 homolog of the yeast Sir2 HDAC in mammalian TPE, and with its H3K9 deacetylase activity (28). Thus, these results suggest a mechanism by which SIRT6 and possibly other proteins may propagate along the mammalian chromosome to silence subtelomeric regions.

Interestingly, our results imply that various chromatin structures and/or mechanisms may be implicated in the telomeric silencing and boundary effects. For instance, distinct telomeric clones display different responses to treatment with agents that affect chromatin-modifying activities. Furthermore, the boundary effect elicited by the CTF1 fusion protein is not always associated with major changes in histone acetylation, as it was rather associated with the incorporation of the histone H2A.Z variant in the insulated gene of one clone. This finding is reminiscent of the previous demonstration that the yeast H2A.Z homolog is capable of synergizing with boundary elements, and that it preferentially locates on insulated telomeric genes (24, 27, 43). Thus, in contrast to the view that the mammalian H2A.Z may have the distinct function of mediating a silent heterochromatin structure (10, 24, 27, 43), our results indicate that it can be associated with gene expression at human telomeres.

What distinguishes telomeric loci where the boundary effect may be associated with histone acetylation or with H2A.Z enrichment is unclear at present, but it may stem from different chromosomal contexts. It has been found that telomeric silencing is often counteracted by HDAC inhibitors in tumor cell lines but not in normal cells (2, 32, 35). While our results are consistent with these observations, they raise the possibility that distinct mechanisms may operate at distinct chromosomal loci, and that the previously observed cell-specific behaviors may also reflect distinct telomeric assay systems.

While the role of the CTCF transcription factor as an enhancer-blocking insulator has been well characterized, the occurrence of mammalian DNA-binding proteins that might mediate chromatin-domain boundary effects has remained elusive. For instance, the USF1 transcription factor binding site present in the chicken HS4 insulator has been proposed to mediate the boundary activity of this epigenomic regulator (41). However, while HS4 can shield transgenes from silencing at chicken telomeres, the USF1 protein was found to be dispensable for this effect (32, 35). Thus, evidence for the long sought DNA-binding activities that may mediate telomeric boundaries in higher eukaryotes could not be obtained. Our results indicate that binding sites for a single transcription factor, or the recruitment of its histone-binding domain by a heterologous DNA-binding activity, suffices to mediate a chromatin domain boundary effect and that it acts to shield transgenes from telomeric silencing effects. In addition, our study provides a means by which very short DNA sequences acting as boundaries may be identified and characterized, opening the way to their use to protect transgenes from silencing effects, for instance by their incorporation in viral or non-viral gene therapy vectors.

### Example 47

### Results

### 4.1. Establishment of a Plasmid-Based Assay System to Quantify the Potency of Genetic Insulator Elements

### System Design

The strategy elaborated by Applicants to address the potential enhancer-blocking activity of genetic elements consists in setting up a two-reporter genes-assay whereby the potency of suspected insulators can be quantified. A series of plasmids were thus designed, containing a combination of two reporter genes: a DsRed gene, used as the assay system internal control, under the control of a Fr-MLV LTR enhancer/promoter, and a GFP gene under the control of a minimal CMV promoter, but also subjected to the influence of the LTR enhancer. The enhancer-blocking activity of suspected insulators interposed between the enhancer and the GFP gene promoter can be revealed by a decrease in the GFP expression, the DsRed expression remaining stable. Drawing a parallel with gene therapy, the DsRed gene can mimic a therapeutic gene whose expression is driven by the retroviral LTR, and the GFP gene can stand fora cellular gene close to the viral vector integration site. A potent insulator flanking the viral vector is able to limit the range of action of the LTR enhancer, shielding the cellular gene from LTR-mediated up-regulation. By analyzing the expression of both reporter genes by flow cytometry, this system provides a standardized quantitative procedure to assess potential insulator elements.

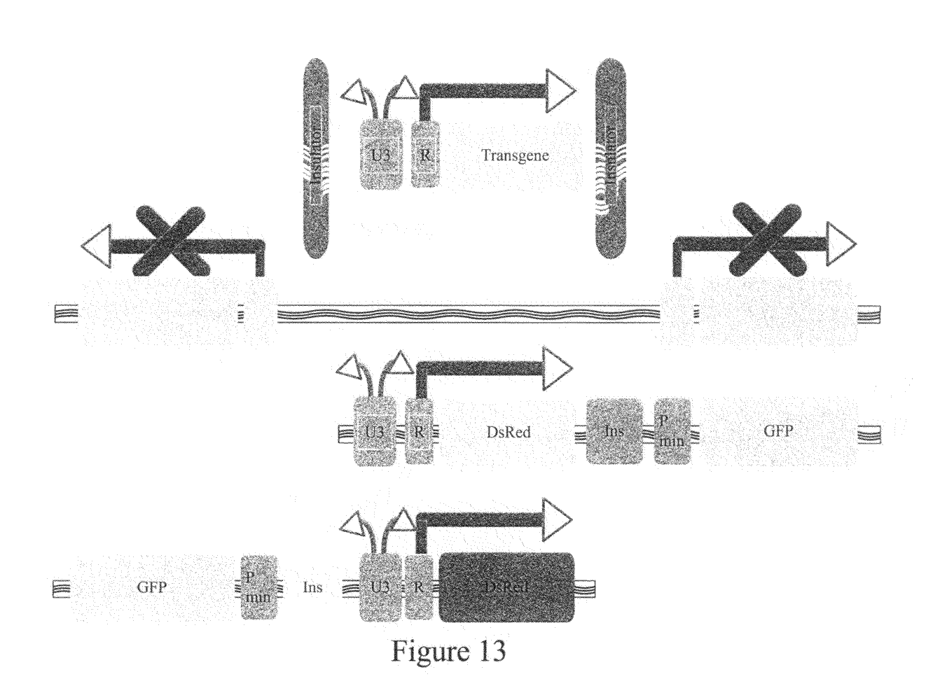

FIG. 13: Design of a Plasmid-Based Screening Procedure for Potential Insulator Elements that Parallels Gene Transfer-Mediated Insertional Activation

In order to mimic events leading to insertional gene activation in a plasmid-based assay system, two types of constructs were designed (FIG. 13). They both contain a DsRed gene under the control of a viral LTR and a GFP gene under the control of a minimal CMV promoter, but they differ by the respective orientation of these genes. Inserting a potent insulator in between the two reporter genes prevents the expression over-activation of the GFP gene without affecting the DsRed expression, as it can shield cellular gene from up-regulation at the site of integration of a viral vector without interfering with transgene expression.

### Available Plasmid Constructs

A first generation assay system was obtained combining different relevant genetic elements in a series of plasmids. The ubiquitous elements to all constructs are the following: one copy of DsRed gene preceded by the viral enhancer/promoter U3R (133 bp long fragment of the LTR extending from base 7708 to 7841 of the helper FB29 Fr-MuLV virus encompassing the viral enhancer (Cohen-Haguenauer, O., Restrepo, L. M., Masset, M., Bayer, J., Dal Cortivo, L., Marolleau, J. P., Benbunan, M., Boiron, M., and Marty, M. (1998). Efficient transduction of hemopoietic CD34+ progenitors of human origin using an original retroviral vector derived from Fr-MuLV-FB29: in vitro assessment. Hum Gene Ther 9, 207-216)) and one copy of GFP gene preceded by a minimal CMV promoter. In addition, in order to insulate the whole construct itself either from the viral enhancer bi-directional activation in transient transfection (circular form of the plasmid) or from a neighboring cellular enhancer at the site of integration in stable transfection (integrated linearized form of the plasmid), a copy of the known cHS4 insulator was inserted at the extremity of each plasmid. Then, in order to validate the ability of that system to reveal insulating activity of genetic elements, known insulators were firstly interposed between the two reporter genes.

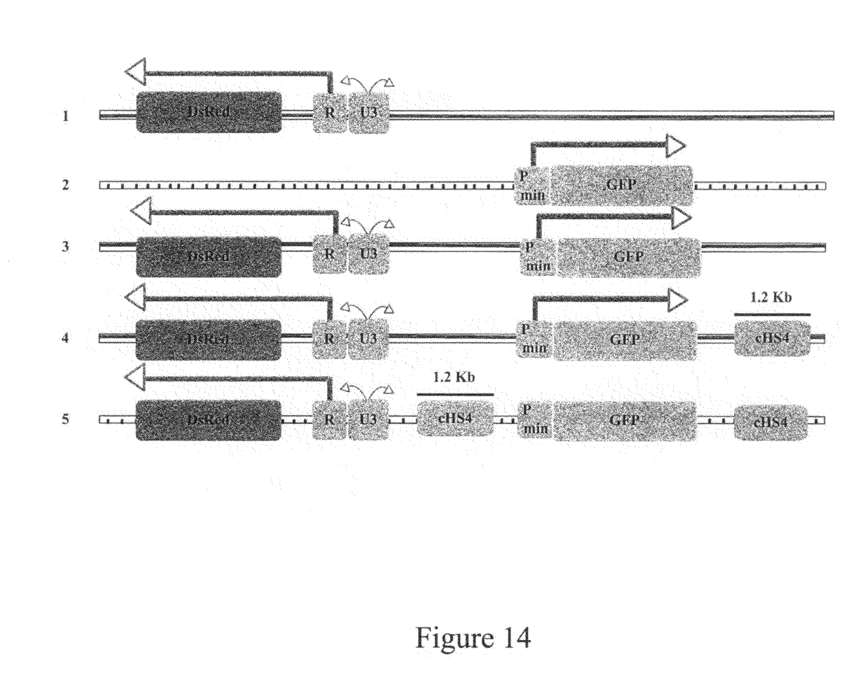

### FIG. 14: First Generation Assay System Plasmids

The following constructs have been made up: 1.pBSU3Rred, 2.pBSGFPmin, 3.pAG1-3-noins, 4.pAG1-3-HS4, 5.pAG1-3-2HS4. The cHS4 inserted is the 1.2 kb fragment. These plasmids are suitable for in vitro experiment in cell culture as well as for in vivo experiments in animal models (FIG. 14).

### 4.2. Validation of the Enhancer-Blocking Ability of the cHS4

As Applicants selected the cHS4 as reference insulator, Applicants firstly tried to reproduce its enhancer-blocker activity using established assays.

The cHS4 Enhancer-Blocking Activity in K562 and HeLa Cells

As part of the chicken β-globin locus, the cHS4 ability to block the extension of the LCR was firstly described in the erythroid cell line K562 (Chung et al., 1993). Using a colony assay based on G418 resistance, Applicants tested whether the cHS4 can insulate a γ-globin promoter/neo reporter gene from a strong β-globin LCR element in K562 cells. From the original pJC5-4 plasmid, Applicants established a series of deriving constructs, which were stably transfected into K562 cells (bottom bars of each pair). The number of G418-resistant colonies was counted 2 to 3 weeks later. Applicants showed that a single copy of the cHS4 was capable of insulating the reporter gene by nearly 4-fold. Transfections of plasmids #2 and #3, containing either no LCR or a cHS4 fragment interposed between the LCR and the promoter, yielded to comparable number of G418-resistant colonies. This result shows that the cHS4 was able to block the LCR-mediated up-regulation of the reporter gene expression. The highest variability was obtained transfecting cells with the construct #1, presumably because of the random integration in the host cell chromatin and making the expression of the reporter gene dependant on any neighboring regulatory structure.

For experimental convenience, Applicants wanted to validate the cHS4 enhancer-blocking activity in HeLa cells (top bars). Using the same approach, the pJC5-4 deriving constructs were stably transfected in HeLa cells. The cHS4 was able to insulate the reporter gene from the LCR activation of nearly 15-fold.

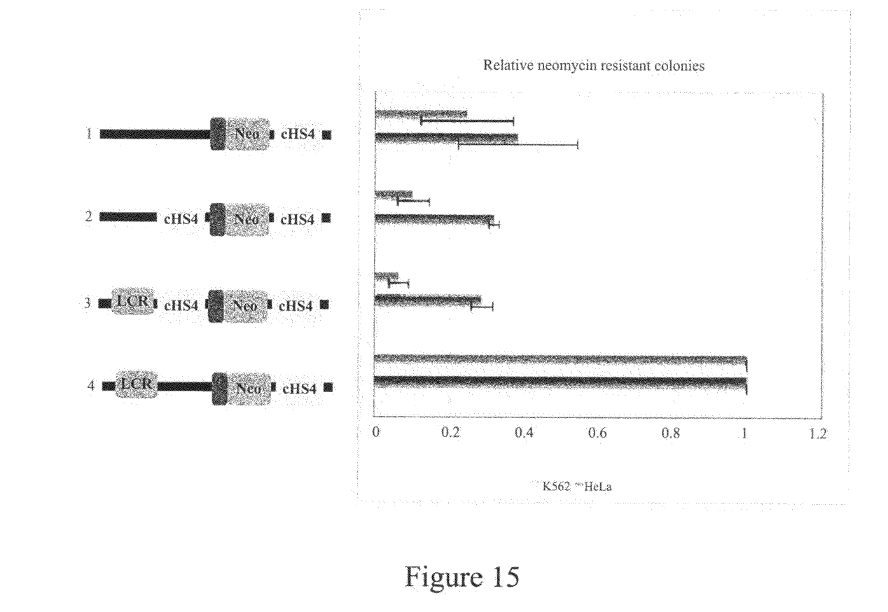

### FIG. 15: Comparison of the cHS4 Insulator Effect in HeLa and K562 Cells

From the pJC5-4 (3) plasmid containing 2 copies of the cHS4 flanking a γ-globin promoter/neo reporter gene, and a β-globin LCR element, 3 other constructs were made removing the interposed cHS4 (4) or the LCR (2), or even both cHS4 and LCR (1). In order to compare the results obtained in both cell lines, the number of G418-resistant colonies for the construct # 4 was set to 1.0. Red bars refer to HeLa results and blue bars refer to K562 results.

The cHS4 Insulator is Able to Block the Fr-MLV LTR Enhancer

The capacity of the cHS4 to block the Fr-MLV LTR strong enhancer was assessed using the same system in HeLa cells. Additional plasmids were constructed, replacing the β-globin LCR element by the Fr-MLV LTR in both orientations. The cHS4 was shown to insulate the reporter gene by approximately 8-fold.

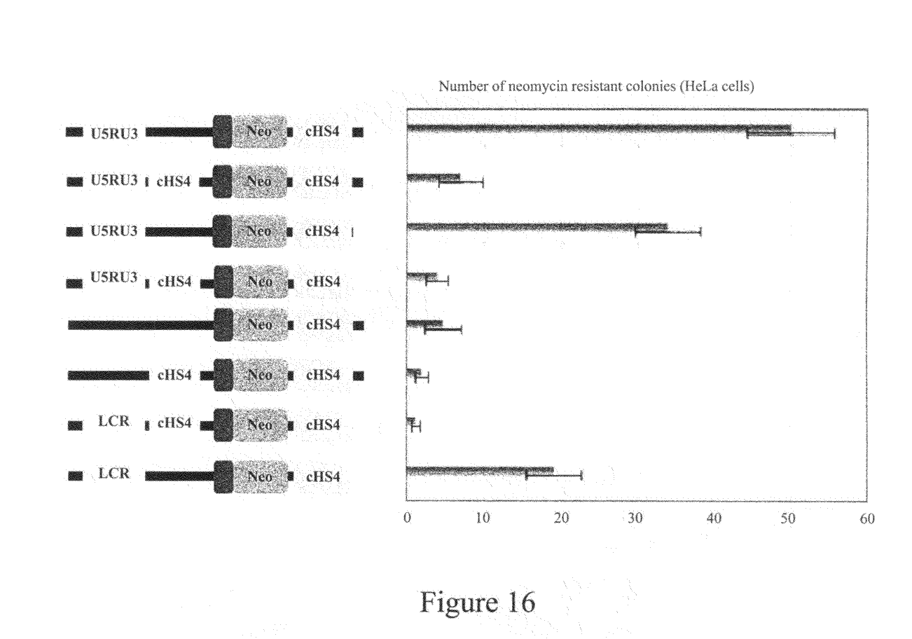

### FIG. 16: Evaluation of cHS4 Ability to Insulate the Fr-MLV LTR Enhancer in HeLa Cells

Four additional constructs were made, each containing the Fr-MLV LTR as enhancer in either orientation (U3RU5 or U5RU3) and with or without a copy of the 1.2 kb cHS4 inserted upstream of the β-globin gene.

### 4.3. Assay System and Results

The cHS4-Mediated Insulation of the Fr-MLV LTR Enhancer in the Two-Reporter Genes-Assay

The constructs described in FIG. 14 were used to transfect transiently HeLa cells. The expression of both DsRed and GFP was analyzed by FACS. The first step was to test the ability of that system to point up the cHS4 enhancer-blocking property, as the cHS4 was shown to function as enhancer-blocker in transient transfection experiments (Recillas-Targa et al., 1999). Applicants showed that the cHS4 was able to block the communication between the viral enhancer and the GFP gene when interposed. Focusing on GFP-positive cells, only less than 2% of the total cell population expressed GFP with the cHS4, as compared to almost 40% without it.

### FIG. 17: FACS Analysis of the cHS4 Insulator Effect in an Enhancer Blocking Assay

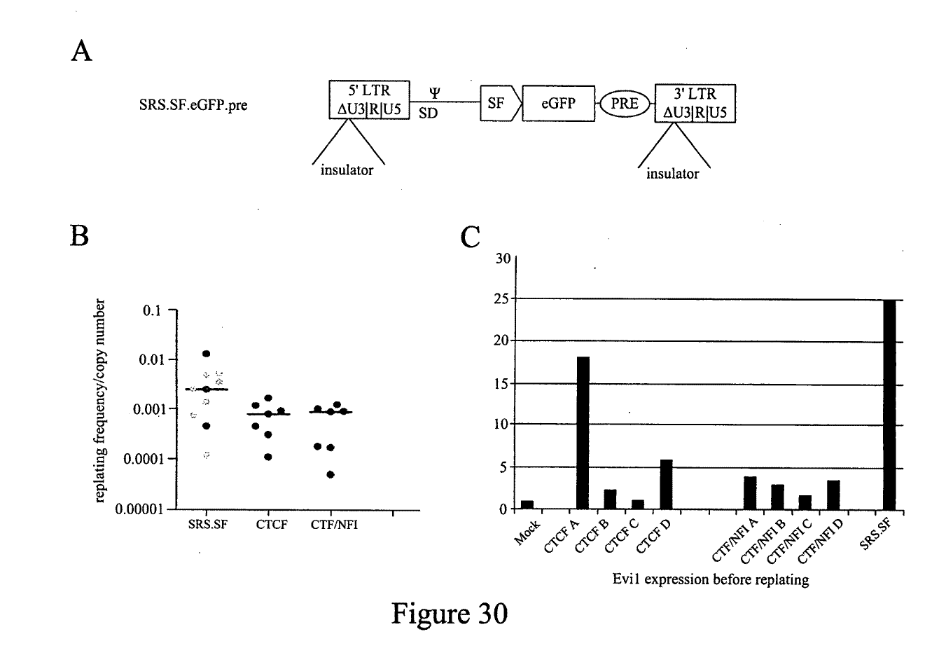

HeLa cells were transfected with pAG1-3-HS4 (left panel) or pAG1-3-2HS4 (right panel) constructs (respectively constructs #4 and #5 on FIG. 30), and 100′000 cells were analyzed 48 hours later. The pAG1-3-2HS4 contains a cHS4 inserted between the viral enhancer and the promoter of the GFP gene. The quadrants have been adjusted to obtain 99% of non-transfected cells in the bottom-left region. Numbers in each quadrant correspond to the percentage of cells in the designated region.

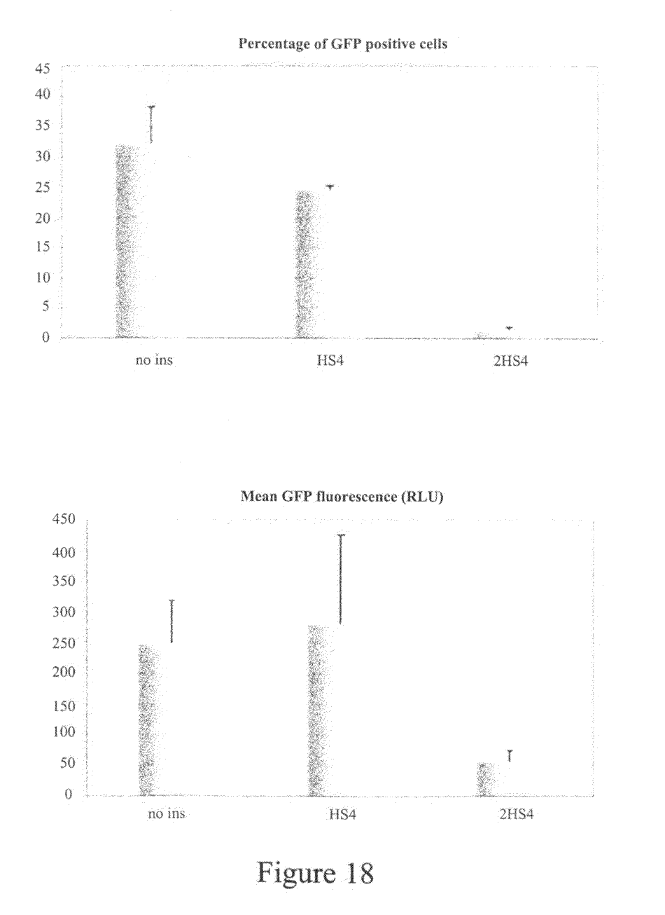

However, very few DsRed-positive cells were detected in this assay. It was hypothesized that the fluorescence intensity of GFP was much higher than DsRed's. Setting aside the unquantifiable DsRed gene expression, decreasing numbers of GFP-positive cells and levels of fluorescence revealed the cHS4 insulator effect. As shown in FIG. 18, interposing the cHS4 between the LTR and the GFP gene leads to a decrease of GFP-positive cells of nearly 20-fold and a decrease of the mean fluorescence of GFP-positive cells of approximately 5-fold (2HS4). Interestingly, the presence of a cHS4 fragment at the extremity of the GFP gene seems to slightly decrease its level of expression (HS4), suggesting that the presence of the cHS4 at the 3′ end of the DsRed gene might have also decreased its expression level.

### FIG. 18: Semi-Quantitative Analysis of the cHS4 Insulator Effect

HeLa cell populations transiently transfected with constructs containing either no cHS4 (noins) or a copy at the GFP extremity (HS4) or two copies flanking the GFP gene (2HS4) were analyzed by FACS. Percentages of GFP-expressing cells (left panel) and GFP mean fluorescence in RLU (right panel) are plotted for each case.

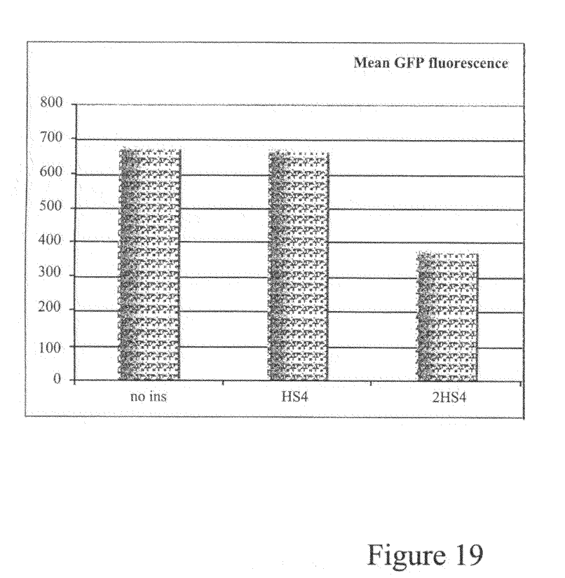

### FIG. 19: Semi-Quantitative Analysis of the CTCF-Binding Sites Insulator Effect

HeLa cells were transiently transfected with either pAG1-3-noins, or pAG1-3-HS4, or pAG1-3-2HS4, or pAG1-3-6CTCF/HS4 (see FIG. 2 for plasmids description) and analyzed by FACS. GFP mean fluorescence in RLU is plotted for each population.

### Assessment of CTF-Mediated Enhancer-Blocking Capacity

Focusing first on the established neomycin-gene expression assay system, Applicants evaluated the functioning of the binding of another transcription factor termed CTF. CTF has been found to mediate a boundary activity when fused to a heterologous GALA protein (Ferrrari et al., 2004). However, a potential activity of the native protein in the boundary or in the insulator assay is not known. FIG. 20 shows that the insertion of 7 CTF binding sites between an enhancer and a minimal promoter driving the expression of a neomycin-resistance gene significantly reduced the occurrence of colonies of neomycin-resistant cells, indicating that these elements may decrease expression of the test gene to levels close to those seen from the construct without any enhancer (pJC-enh/ins).

FIG. 20. Assessment of the insulator activity of multimerized CTF binding sites using the commonly-used neomycin-resistance insulator assay. The number of neomycin resistant colonies were determined as in FIGS. 15 and 16 after 2 weeks of G418 selection of HeLa cells transfected using the indicated reporter constructs. Reporter constructs contain either the 1.2 kb HS4 insulator and/or a 164 base pair element containing 7 CTF binding sites, the ft-globin enhancer (enh) and the neomycin resistance gene expressed from a minimal gamma-globin promoter (neo). pJC vectors (except for pJC-CTF) were described by Chung et al., 74:505-514, 1993.

The insulator activity of CTF sites was further evaluated using the semi-quantitative assay relying on the quantification of GFP fluorescence in flow cytometry. This assay consistently indicated that interposition of the CTF binding sites between the enhancer and GFP reporter gene decreased the average fluorescence of the population of transfected (GFP expressing) cells by approximately three-fold, while it had little or no effect of the efficacy of transfection, as indicated by the similar percentile of GFP+ cells.

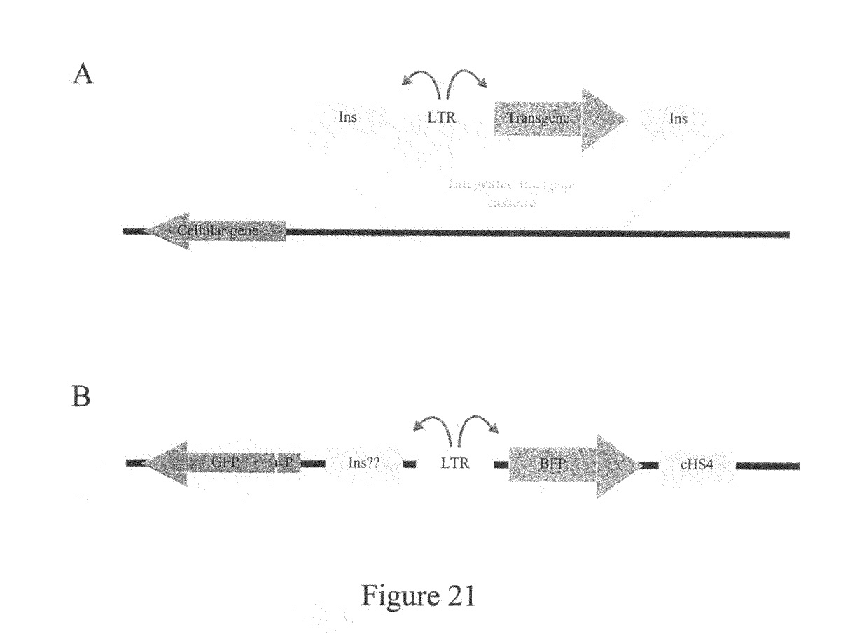

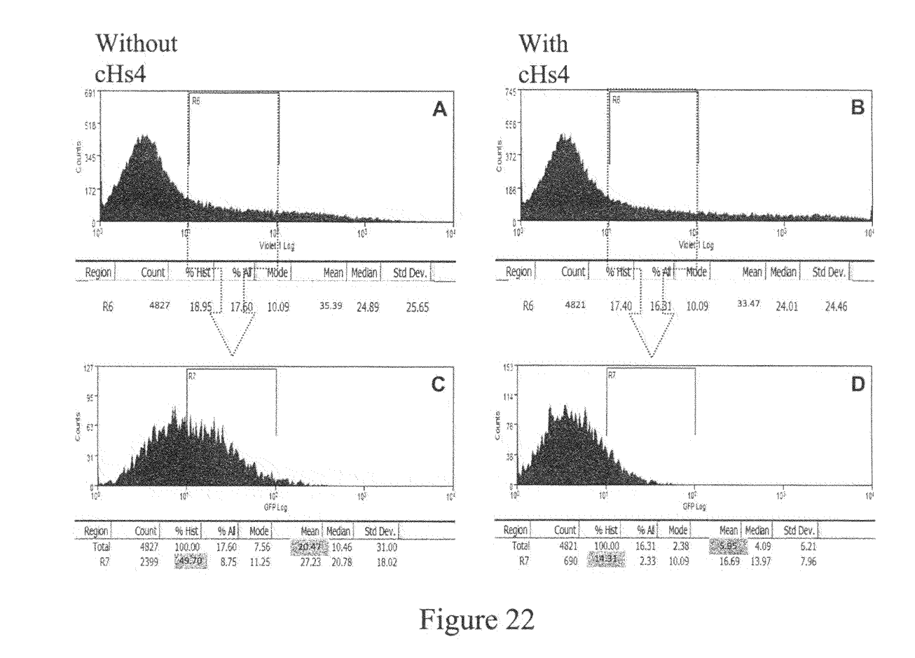

As before, DsRed could not be quantified easily in this assay. Therefore, Applicants turned to another assay where the DsRed gene was removed from the plasmids shown in FIG. 14, and the resulting constructs were co-transfected with a BFP (blue fluorescent protein) expression vector serving as a control for the transfection efficiency. The organization of the elements of the improved insulators screening vectors is shown in FIG. 21. This assay was validated as before with the known cHS4 insulator (FIG. 22).

### FIG. 21: Schematic Illustration of the Improved Plasmid-Based Screening Assay for Potential insulator elements

Panel A is a schematic illustration of the retroviral-mediated integration of a transgene expression cassette into a host cell genome upon viral infection. This cassette contains a transgene under the control of a retroviral LTR and is flanked by a genetic insulator element shielding a cellular gene at the site of integration from LTR-mediated up-regulation. In order to mimic events leading to gene transfer-mediated insertional activation in a plasmid-based assay system (B), plasmid constructs were designed containing a BFP gene under the control of a viral LTR enhancer-promoter and a GFP gene under the control of a minimal CMV promoter (P). Inserting a potent insulator in between the two reporter genes should prevent the expression over-activation of the GFP gene without affecting the BFP expression, as it should shield cellular gene from up-regulation at the site of integration of a viral vector without interfering with transgene expression. In addition, in order to insulate the whole construct itself either from the viral enhancer bi-directional activation in transient transfection (circular form of the plasmid) or from a neighboring cellular enhancer at the site of integration in stable transfection (integrated linearized form of the plasmid), a copy of the cHS4 insulator is inserted at the edge of the reporter gene expression cassettes

### FIG. 22: Comparison of FACS Profiles of BFP and GFP Expression Levels of HeLa Cells Transfected with the Improved Insulator Assay Constructs Either with or without the cHS4

HeLa cells were transiently transfected with insulator assay constructs described in FIG. 22, with (right) or without (left) a copy of the cHS4 interposed in between the enhancer and the promoter of the GFP gene. Profiles of BFP expression over total cell population are similar in each case (A and B). Same number of BFP positive cells with expression levels comprised between 10¹ and 10² RLU were analyzed for their GFP expression levels (C and D respectively). In the presence of the cHS4 (D), the GFP profile is shifted to the left in comparison with the profile in the absence of cHS4 (C). For similar levels of BFP, cells express significantly lower levels of GFP when the cHS4 is interposed in between the enhancer and the promoter driving the GFP gene.

The next assessed systematically series of potential insulator elements using the quantitative assay described above and shown in FIG. 26.

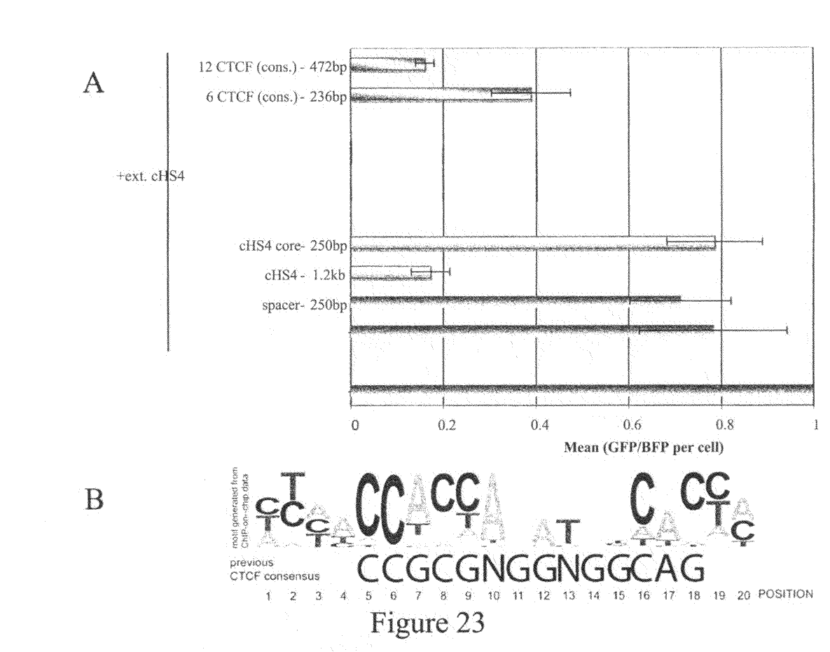

To identify stronger enhancer-blocking genetic elements, another version was synthesized containing six repeats of the consensus binding site based on the CTCF-binding motive defined from ChIP-on-chip experiments (Kim et al., 2007) [FIG. 23 B].

FIG. 23: Quantitative analysis of CTCF binding sites insulator activity compared to the cHS4. HeLa cells were transiently transfected with constructs described in FIG. 13 and FACS analyses were performed 48 hours after transfection (A). The mean of GFP expression normalized to BFP expression per cell is plotted for each construct. All constructs contain a copy of the cHS4 insulator at the edge of the reporter genes expression cassette at an external position except the control construct, at the very bottom. Elements interposed in between the enhancer and the promoter of the GFP gene are indicated on the Y-axis as well as their respective size. Assessed elements are a series of binding sites for CTCF containing the consensus binding site (cons.) based on the CTCF-binding motive defined from ChIP-on-chip experiments (Kim et al., 2007) (B); and they are compared to the cHS4 and the cHS4 core elements for their enhancer-blocking activities.

Linkers between each binding sites were added up to the size of a footprint of CTCF protein and their sequence was randomly defined in order to limit the repetitive elements. Doubling this element gave rise to a sequence of 12 binding sites for CTCF, also assessed in the insulator assay.

Consensus binding sites for CTCF were able to show the same activity, with no significant improvement. However, when doubling the number of binding sites, from 6 to 12, the insulator activity obtained became comparable to the one of the full length cHS4 with a genetic element of hardly more than one third of its length [FIG. 23 A].

### Analysis of Enhancer-Blocking Activity of the CTF Element

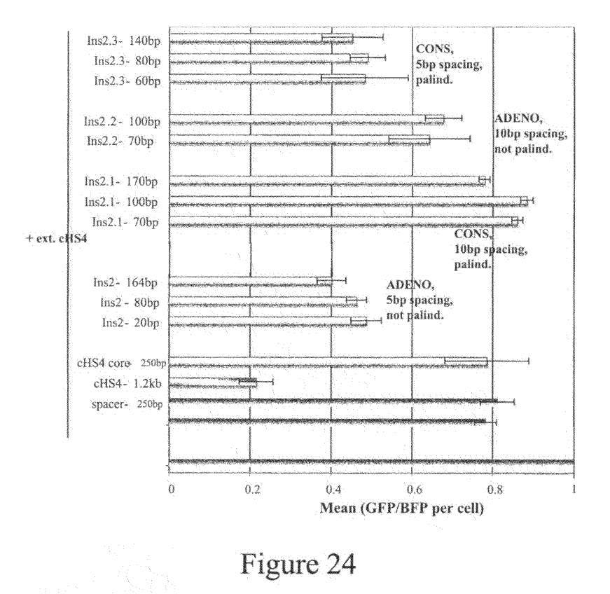

Native CTF binding site are called INS2 in the following text and figures, whereas its derivatives are called INS2.X, where X represents the variant number. INS2 showed comparable insulator activity with binding sites for CTCF, i.e. approximately half of the full length cHS4 [FIG. 24]. Interestingly, the insulator activity observed is proportional to the number of binding sites for INS2. One binding site (20 bp element) seems to be responsible for most of the effect. Possible explanations may be that the cellular levels of INS2 proteins would be already limiting with one binding site or that the additional binding sites are not optimally occupied or that just one protein suffices for the effect.

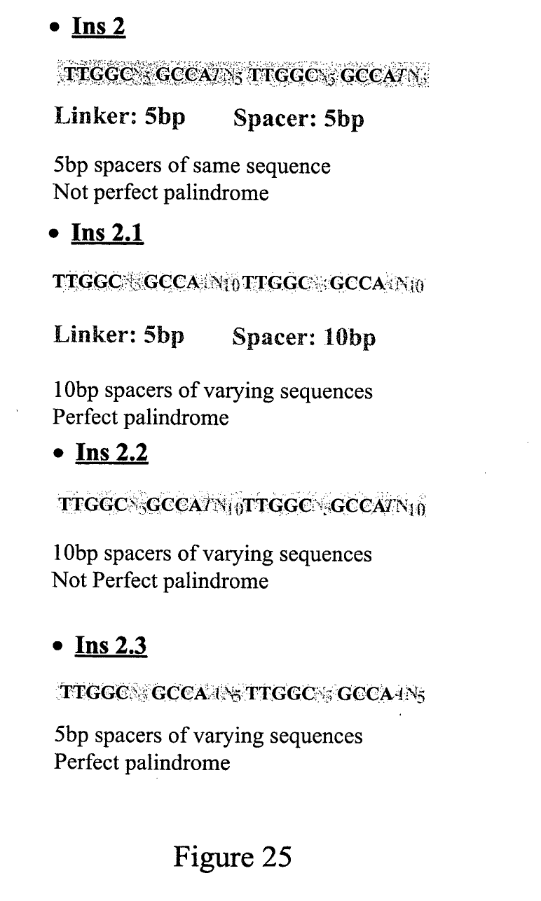

The first variant of Ins2 element is the Ins2.1 element, which is composed of the consensus binding sites for INS2 and has been designed in order to have the size of a footprint (20 bp) of INS2 protein at each binding site (with a spacing of 10 bp between individual sites) [FIG. 25]. This series of binding sites do not show enhancer-blocking activity, even combining 7 binding sites. Taking the size of a footprint for each binding sites actually places the nucleotides recognized by the DNA binding domain of INS2 on the same side of the DNA molecule possibly impairing proper binding of two INS2 molecules at a time. To address this point, variants Ins2.2 and In2.1 have been assessed. As expected, Ins2.2 containing native binding sites but with the same spacing as Ins2.1, and it failed to show potent enhancer-blocking activity, whereas Ins2.3 element, containing the consensus binding site for INS2 and the same type of spacing than Ins2 element reproduce similar insulator activity than Ins2 [FIG. 24]. Even though Ins2.3 does not show better insulator activity than Ins2, it contains much less repeated sequences and should thus exhibit better compatibility with retroviral vectors.

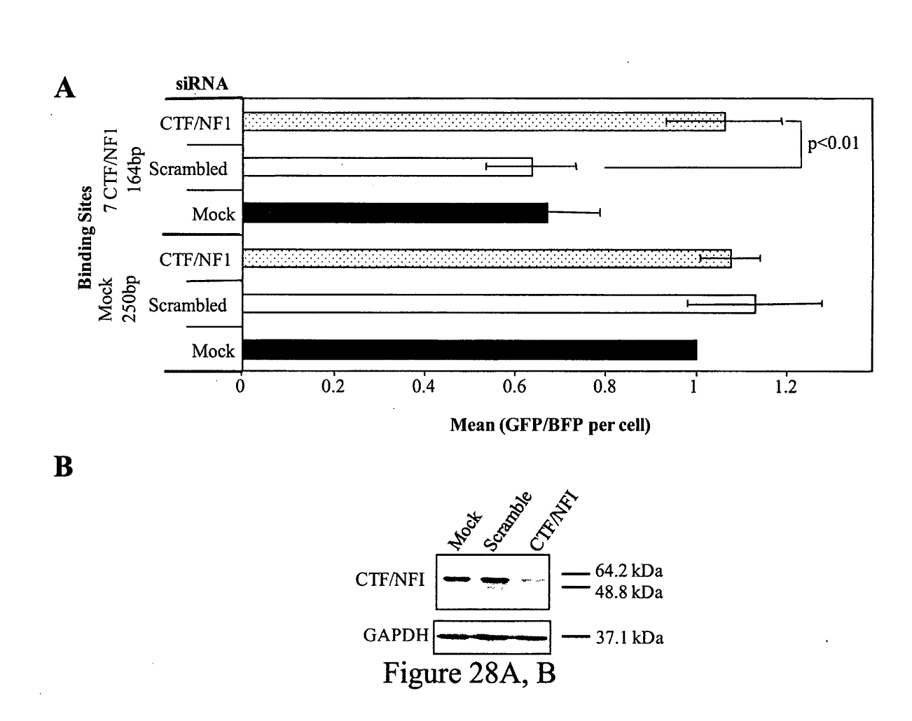

In order to further address the insulator activity of these insulators to the INS2 proteins themselves (i.e. CTF/NF1 proteins) and to exclude possible recruitment of other factors that could contribute to the effect observed, INS2 expression was knocked down in HeLa cells before transfection of the insulator assay constructs using siRNA. After mock transfection or transfection of a scramble siRNA, no modification of Ins2 insulator effect was observed although it was completely abolished after knock-down of the CTF proteins (FIG. 28).

The present invention describes novel types of assays for insulator elements based on the combination of distinct fluorescent-expressing proteins, some being insulated by genetic elements while other act as control for the efficacy of transgene expression. These new assay principles and vectors were validated using a known insulator element (cHS4). Applicants' results further imply that the two reporter genes can be either on the same plasmid or co-transfected on separate plasmids.

FIG. 24: Quantitative analysis of Ins2 binding sites insulator activity compared to the cHS4. HeLa cells were transiently transfected with constructs described in FIGS. 25 and 26A and FACS analyses were performed 48 hours after transfection. The mean of GFP expression normalized to BFP expression per cell is plotted for each construct. All constructs contain a copy of the cHS4 insulator at the edge of the reporter genes expression cassette at an external position except the control construct, at the very bottom. Elements interposed in between the enhancer and the promoter of the GFP gene are indicated on the Y-axis as well as their respective size. Assessed elements are a series of binding sites for Ins2, either native (Ins2, Ins2.2) or containing the consensus Ins2-binding site (Ins2.1, Ins 2.3) deduced from SELEX-SAGE screening experiments (Roulet et al., 2002) as well as other sequences. Different types of Ins2 elements were synthesized (Ins2.1, Ins2.2, Ins 2.3), varying from one another by the spacing sequences surrounding the binding sites, as well as various length-variants for each sub-type.

### FIG. 25: Description of Ins2 Binding Site Derivatives

Using a combination of conventional antibiotic resistance assay and of the new assay, Applicants identify CTF and CTCF binding sites as short insulator elements capable of shielding a gene from the activation mediated by a potent LTR enhancer element nearby on the DNA. Applicants demonstrate that the insulator activity is preserved when the multimerized elements are imbedded in a deleted viral LTR, in a context similar to the one occurring after integration of the insulator element in a viral gene therapy vector. Applicants therefore conclude that these elements, alone or in various combinations, can be used to generate safer gene therapy vector. These elements can allow efficient expression of the therapeutic gene borne by the viral vector while preventing the activation of cellular genes neighboring the site of vector integration, including genes which activation may lead to sever adverse effects in patients such as cancers.

Various modifications and combinations of these elements are tested, as well as their combination with the binding sites of other proteins that show insulator effect, including the sea urchin arylsulfatase insulator (Hino et al., 2006), and/or boundary elements such as those binding the transcription factors USF1 and USF2 (West et al., 2004). This can be done by combining different binding sites in homopolymers or heteropolymers, making point mutations to optimize the sequence, changing the number of combined binding sites, or combinations thereof, to obtain most potent insulator elements.

Elements showing potent insulation in vitro and characteristics suitable for gene therapy vectors are then assessed in vivo. Their insulator effect can be validated by in situ electroporation of mouse muscle using the plasmid-based assay system (McMahon et al., 2001). The safety of the new vectors can also be evaluated in animal models to follow possible tumor formation (Montini et al., 2006) or in vitro to follow clonal cell expansion as a marker for tumor formation (Schambach et al., 2006a; Modlich et al., 2006).

### Example 5

### Material and Methods

### 5.1 Plasmid Vectors and Insulator Sequences

The plasmid constructs described in FIG. 26A were constructed from the pJC5-4 plasmid kindly provided by Dr. Gary Felsenfeld (Physical Chemistry Section, National Institutes of Health, Laboratory of Molecular Biology, Bethesda, Md., USA) (Chung et al., 1993), which originally contains the following elements in a pGEMZ backbone (Promega): the mouse 5′HS2 LCR, the human ^{A}γ-globin promoter linked to the neomycin (G418) resistance gene and flanked by one copy of the 1.2 kb cHS4 insulator. The 5′HS2 LCR was substituted by the Friend-murine leukemia virus (Fr-MuLV, FB29 strain, Cohen-Haguenauer et al., 1998) LTR either in its 5′-3′ native orientation or in the inverted orientation. The cHS4 was deleted by restriction digestion and re-ligation of the vector when indicated.

The plasmid constructs described in FIG. 26B were obtained as follows. The EGFP gene expressed from a minimal CMV promoter was PCR amplified from a pcDNA3-EGFP plasmid excluding the CMV enhancer. The EBFP gene was PCR amplified from the pEBFP-NI plasmid (Clontech). Both reporters were subcloned in a pBS2-SKP (Stratagene). The Fr-MuLV LTR was inserted upstream from the EBFP gene such that transcription from the LTR is directed towards EBFP, and a copy of the 1.2 kb cHS4 was inserted downstream from the EGFP gene. Insulator sequences were inserted between the Fr-MuLV LTR and the minimal CMV promoter driving GFP expression. The 250 bp cHS4 core was PCR amplified from the full-length cHS4 (GenBank accession number: U78775.2, amplification from position 1 to 250). A series of neutral DNA spacers of various lengths were PCR amplified from the mouse utrophin cDNA (GenBank accession number: BC062163.1, amplifications form position 355 to positions 605 and 1555).

Binding sites for CTCF and CTF/NFI were obtained by annealing complementary oligonucleotides ended by cohesive and compatible extremities (XbaI-SpeI), which were phosphorylated and multimerized by ligation to obtain multiple binding sites. Native CTCF binding sites refer to the BEAD-A and the FII sequences (Bell et al., 1999). Consensus CTCF binding sites correspond to direct repeats of the consensus binding motif (Kim et al., 2007) separated from one another with spacers up to the size of a native binding site (40 bp). CTF/NFI binding sites are composed of direct repeats of the CTF/NFI binding site from the adenovirus type II origin of replication isolated from the pNF7CAT plasmid (Tarapore et al., 1997). The consensus CTF/NF1 binding site was obtained from SELEX-SAGE experiments (Roulet et al., 2002). Sequences of the spacers separating two adjacent CTCF or CTF/NFI consensus binding sites were randomly chosen. The complete sequences of the synthetic insulators are detailed in the Supplementary Materials and Methods section.

### 5.2 DNA Transfection of K562 and Colony Assay

DNA transfection of K562 cells were performed as previously described (Chung et al., 1993). Briefly, 10⁷ cells were electroporated in cold PBS with 0.25 μg of linearized DNA (Bio-Rad Gene Pulser II, 200 V, 960 μF). To generate neomycin-resistant colonies, transfected cells were grown in semi-solid medium composed of Iscove's modified Dulbecco's medium (ATCC), 10% fetal bovine serum (GIBCO), 0.3% cell culture agar (Sigma) and 500 μg/mL G418 (GIBCO). Resistant colonies were counted after 2 to 3 weeks of selection for G418 resistance.

### 5.3 DNA Transfection of HeLa Cells and Flow Cytometry Analyses

HeLa cells were transfected using FuGENE 6 reagent (Roche Diagnostics) according to the manufacturer's recommendations. Equimolar amounts of the different plasmids were transfected in each experiment (using the pBS2-SKP backbone plasmid as carrier). Circular plasmids were used for transient transfections, while plasmids were linearized before stable transfections. To obtain stable populations, the reporter constructs were co-transfected with a puromycin resistance-encoding plasmid (pPUR, Clontech) with a molar ratio of 10:1 and cells were grown in Dulbecco's modified Eagle medium containing 10% fetal bovine serum and 0.5 μg/mL puromycin (all from GIBCO). Fluorescence analyses were acquired on the FACS Cyan (Dakocytomation) with the settings of 450 V on the SSC channel, 340 V for the GFP and 450 V for the BFP. Data analysis of the double-reporter assay consisted in normalizing the GFP fluorescence to the BFP fluorescence for each cell and averaging these values over the total cell population. FACS analyses were performed 48 hours after transfection for transient expression and after 2 to 3 weeks of selection post-transfection for stable expression. Data processing was performed using the FlowJo software.

### 5.4 siRNA Experiment

Where indicated, HeLa cells were transfected with 50 nM siRNA targeting the mRNA of all CTF/NFI isoforms (sc-43561, Santa Cruz) or with a non-targeting control (scrambled siRNA, sc-37007, Santa Cruz) using Oligofectamine (Invitrogen) according to the manufacturer's recommendations. Cells were transfected with the double-reporter construct 24 hours later and FACS analyses were performed 48 hours after DNA transfection as described above.

5.5 Western Blot Analysis

Western blotting was done following standard protocols: protein extracts from a defined number of cells were separated in SDS-polyacrylamide gels (7.5% polyacrylamide in running gel), transferred to nitrocellulose membrane (Schleicher and Schuell), and incubated with the primary antibodies: anti-NFI (H-300, Santa Cruz, dilution 1:200) applied overnight and anti-GAPDH (sc-32233, Santa Cruz) applied 2 hours after blocking of the membrane in 5% dried-milk (in PBS). After incubation with a goat anti-rabbit horseradish peroxidase-coupled secondary antibody (SIGMA) or a goat anti-mouse horseradish peroxidase-coupled secondary antibody (Jackson Lab), the membrane was subjected to the enhanced chemiluminescence's immunodetection (Amersham). Bands intensity was quantified using ImageJ software.

### 5.6 Retroviral Vector Design and Titer Determination

The gammaretroviral self-inactivating (SEN) vector has been described previously (Schambach et al., 2006b). The insulator sequences were inserted into the NheI site of the 3′ ΔU3 region, which will be copied into the 5′ LTR after reverse transcription, and thus results in a design flanking the introduced expression cassette.

Gammaretroviral supernatant production was performed using 293T cells as previously described, with the co-expression of ecotropic envelope proteins (Schambach et al., 2006a; Schambach et al., 2006b). Cells were maintained in Dulbecco's modified Eagles Medium (DMEM) supplemented with 10% FCS, 100 U/ml penicillin/streptomycin, and 2 mM glutamine. Viral titers, determined on SC-1 cells by flow cytometry, were in the range of 5×10⁶ to 2×10⁷ IU/mL in unconcentrated supernatants.

### 5.7 Isolation of Lineage-Negative Bone Marrow Cells and Retroviral Transduction, In Vitro Immortalization (IVIM) Assay and TaqMan Real-Time PCR Analysis

Lineage-negative (Lin-) bone marrow (BM) cells of untreated C57BL6/J mice (Charles River Laboratories, Wilmington, Mass., USA) were transduced as previously described (Li et al., 2003). Briefly, Lin-cells were isolated from complete BM by magnetic sorting using lineage-specific antibodies (Lineage Cell depletion kit, Miltenyi, Bergisch Gladbach, Germany) and were cryopreserved in aliquots. Before retroviral transduction, Lin-BM cells were prestimulated for 2 days in Stem Span medium (Stem Cell Technologies) containing 50 ng/ml mSCF, 100 ng/ml hFlt-3 ligand, 100 ng/ml hIL-11, 10 ng/ml mIL-3 (PeproTech, Heidelberg, Germany), 1% penicillin/streptomycin, and 2 mM glutamine at a density of 1-5×105 cells/ml. Cells were transduced on two to three following days (days 3, 4 and 5, FIG. 28) using 10⁵ cells and a multiplicity of infection (MOI) of 10 per transduction. Virus preloading was carried out on RetroNectin-coated (10 μg/cm2; TaKaRa, Otsu, Japan) suspension culture dishes by spinoculation for 30 minutes at 4° C. 1×10⁵ cells were seeded into 500 μl medium, which was completed with 250 μl medium incrementson the following days, so that the final culture volume was 1.25 ml on day 6. DNA samples for real-time PCR analysis (copy number) and flow cytometry (FACSCalibur, Becton-Dickinson, Heidelberg, Germany) were taken four days after the last transduction.

The in vitro immortalisation (NIM) assay was performed as previously described (Modlich et al., 2006). Briefly, after retroviral transduction, BM cells were expanded as mass cultures for 2 weeks in IMDM containing 50 ng/ml mSCF, 100 ng/ml hFlt-3 ligand, 100 ng/ml hIL-11, 10 ng/ml mIL-3, 10% FCS, 1% penicillin/streptomycin, and 2 mM glutamine. During this time, cell density was adjusted to 5×105 cells/ml every 3 days. After mass culture expansion for 14 days, BM cells were plated into 96-well plates at a density of 100 cells/well or 10 cells/well (Modlich et al., 2006). Two weeks later the positive wells were counted, and the frequency of replating cells was calculated based on Poisson statistics using L-Calc software (Stem Cell Technologies, Vancouver, BC, Canada). Selected clones were expanded for further characterization.

Quantitative PCR was performed on an Applied Biosystems 7300 Real-Time PCR System (Foster City, Calif., USA) using the Quantitect SYBR Green Kit (Qiagen, Hilden, Germany) as previously described (Modlich et al., 2009). To measure vector copy numbers, the vector insertions were detected by the wPre element and normalized to the signal of the housekeeping gene Flk (wPRE for primer: GAG GAG TTG TGG CCC TT GT, wPRE rev. primer: TGA CAG GTG GTG GCA ATG CC, flk.intron for: GGT TFC AAT GTC CCG TAT CCTT, flk.intron rev: CTT TGC CCC AGT CCC AGT TA). Results were quantified using the efficiency corrected comparative CT method. To evaluate mRNA expression, RNA was extracted from expanded clones using RNAzol (WAK chemicals, Steinbach, Germany) and the RNAeasy micro kit (Qiagen, Hilden, Germany). Reverse transcription was performed with 0.5 to 2 μg RNA using PowerScript MLV reverse transcriptase (Becton Dickinson), and real-time PCR for Evi1 expression as described (Modlich et al., 2008).

### Example 6

### Results

### 6.1 Design of a Quantitative Enhancer-Blocking Insulator Activity Assay

Using an assay based on the transfection of a plasmid conferring neomycin (G418) resistance (FIG. 26A), we firstly assessed the capacity of the cHS4 to insulate a γ-globin promoter/neo reporter gene from activation by the mouse 5′HS2 locus control region (LCR). K562 cells were transfected with linear forms of the plasmid and the number of G418-resistant colonies was counted after 2 to 3 weeks of culture under antibiotic selection. Presence of the 5′HS2 LCR significantly increased the occurrence of G418-resistant colonies in the human erythroleukemia K562 cell line, while the cHS4 insulator was able to block the LCR-mediated up-regulation of the reporter gene when interposed between the enhancer and the promoter, as expected from prior work (FIG. 26C left panel) (Moon and Ley, 1990) (Chung et al., 1993). The original pJC5-4 construct kindly provided by Dr. G. Felsenfeld (FIG. 26A), was used to construct derivatives in which the enhancer and/or the interposed copy of the cHS4 insulator (cHS4 int.) were deleted. The cHS4 insulator was shown to decrease the number of G418-resistant colonies by nearly 4-fold and to fully prevent the LCR-mediated up-regulation to levels comparable to those observed from the γ-globin promoter without LCR and insulator. Similar results were obtained from the transfection of HeLa cells (FIG. 26C, left panel).

The ability of the cHS4 insulator to block activation from the potent enhancer present on the Friend-murine leukemia virus long terminal repeat (Fr-MuLV LTR) was then similarly assessed in HeLa cells. Substitution of the β-globin LCR by the Fr-MuLV LTR in either orientation strongly increased the occurrence of resistant colonies (FIG. 26C, right panel). Although the Fr-MuLV LTR proved to be a much stronger enhancer than the β-globin LCR in this cell type, the cHS4 was able to decrease the growth of resistant colonies nearly 8-fold when interposed between the enhancer and the promoter of the reporter construct, yielding levels similar to those obtained in the absence of any enhancer.

The insulator assay based on resistant colony counting remains semi-quantitative, and it does not clearly distinguish insulating activities from direct effects on the expression of the reporter gene. Therefore, we designed a two-reporter gene assay whereby the potency of enhancer-blocker insulators can be quantified, and in which polar insulating activities can be distinguished from enhancer inhibition or from global gene silencing effects. As compared with the previous set-up, a CMV promoter/GFP gene cassette substituted the y-globin promoter/neo reporter, and a blue fluorescent protein (BFP) reference gene was inserted into the plasmid so as to be expressed from the enhancer and promoter located on the viral LTR (FIG. 26 B). Changes in the expression of the reporter and reference genes were assessed by cytofluorometry from the GFP and BFP fluorescence profiles of single cells within populations of transiently transfected HeLa cells. Interposition of a copy of an enhancer-blocking insulator between the CMV minimal promoter driving the GFP gene and the LTR enhancer is then expected to decrease GFP expression but not that of BFP, as illustrated in FIG. 26D. The interposed cHS4 (cHS4 int.) induced a significant drop in GFP levels, as revealed by a shift of the whole GFP fluorescence profile towards lower expression levels, whereas BFP profiles were not affected by the insulator (FIG. 26D left and middle panels, respectively). Single cell imaging of the relative GFP and BFP levels showed an homogeneous decrease of GFP expression relative to BFP (FIG. 26D, right panel). These results confirmed a robust enhancer-blocking activity of the full-length cHS4, i.e. the ability to disrupt enhancer-promoter communication when interposed between the two elements without neither altering the enhancer's ability to activate another promoter nor silencing the adjacent promoter.

This effect was quantified by normalizing the GFP fluorescence to that of BFP in each cell of the population to differentiate GFP expression variations due to the insulator effect from differences in expression levels that result from the variability in transfection efficiency. When interposed, the cHS4 induced a significant decrease in GFP expression down to approximately 20% of the enhancer-activated level (FIG. 26E). Only a small proportion of this effect (approximately 20%) may be attributed to the increased distance between the enhancer and the promoter driving the GFP, as interposition of a 1.2 kb-long neutral fragment, portion of the utrophin gene, had little effect on the GFP/BFP fluorescence ratio. Insertion of a single copy of the cHS4 core of 250 bp did not result in a significant insulator effect in this assay.

### 6.2 Optimized CTCF and CTF/NFI Binding Sites Display Potent Enhancer-Blocking Activities

Applicants designed a composite element made of 3 copies of the CTCF FIE binding site in the cHS4 insulator and 3 copies of a homologous site in the BEAD-1 element from the human T cell receptor α/δ locus (Bell et al., 1999) (FIG. 27A). This element showed half of the insulator activity of full-length cHS4, despite its shorter size (270 bp). This element has also displayed high insulating activity when embedded within an inactivated LTR, thus mimicking the context in which the insulator would be in a retroviral or lentiviral vector (FIG. 27B). Another version of this element was synthesized to contain 6 repeats of the consensus binding site based on the CTCF-binding motive defined from ChIP-on-chip experiments (Kim et al., 2007) (CTCF cons; FIG. 27A). Linkers were added between each binding sites to make up for the size of a CTCF footprint and the linker sequences were randomly defined in order to limit the occurrence of repetitive DNA sequences. Six copies of this consensus element already showed significant activity. However, doubling the number of consensus biding sites, giving rise to 12 consecutive binding sites, fully reproduced the insulation effect the entire 1.2 kb cHS4 element (FIG. 27B).

A series of binding sites for CTF/NF1 proteins were also evaluated for a possible enhancer-blocking activity using the double reporter-assay system. Binding sites derivatives were generated to alter the nature of the last base of the binding site, either a T like in the native CTF/NF1 binding site from the adenovirus type II origin of replication (referred to as adeno.), or an A to fit the consensus CTF/NF1 binding site, as obtained from SELEX-SAGE experiments (referred to as cons.). The length of the spacing between two adjacent binding sites was also altered, with either 5 or 10 base-pairs so as to orient binding sites on similar or opposite sides of the DNA double helix (FIG. 27A and Supplementary Table 2). Adeno. binding sites with a spacing of 5 bp appeared to be the most potent elements, even when embedded within a LTR. Unlike for CTCF sites, decreasing the number of repeats did not lead to a significant loss of insulating activity, as even as a single binding site of 20 bp still mediated approximately half of the insulating effect seen with the full-length cHS4 (FIG. 27C). Even though the 10 bp spacing should provide sufficient length to accommodate all directly contacted nucleotides within the binding sites (Roulet et al., 2002), the spacing of 5 bp gave the best insulating activity for all of the tested CTF/NFI-binding sequences. This may result from CTF/NFI adjacent binding sites lying on opposite sides of the DNA double helix, which may limit steric hindrance effects.

To ascertain that CTCF as well as CTF/NFI binding sites may also display an enhancer-blocking activity in the context of a native chromatin structure and in a chromosomal environment, the assays were also performed in stable HeLa cell transfections. The insulating window of the full-length cHS4 was reduced to approximately 2.5 fold decrease of the reporter gene expression, while the cHS4 core showed no activity as before (FIG. 27D).

In order to directly identify the protein responsible for the CTF/NF1 binding site-mediated enhancer-blocking activity, HeLa cells were co-transfected with siRNA targeting all CTF/NF1 isoforms, and the insulator assay was performed with constructs containing either a neutral spacer of 250 bp or the most active combination of CTF/NF1 binding sites. The enhancer-blocking activity of CTF/NF1 was observed with mock-transfected cells or with cells transfected with a scrambled non-specific siRNA (FIG. 28A). However, insulator activity was entirely lost upon an 80% knock-down of CTF/NF 1 protein levels with the specific siRNA, demonstrating the role of the CTF/NF1 transcription factors family as enhancer-blocking insulators in mammalian cells (FIGS. 28A and 28B).

### 6.3 CTF/NFI Binding Sites Also Show Barrier Activities

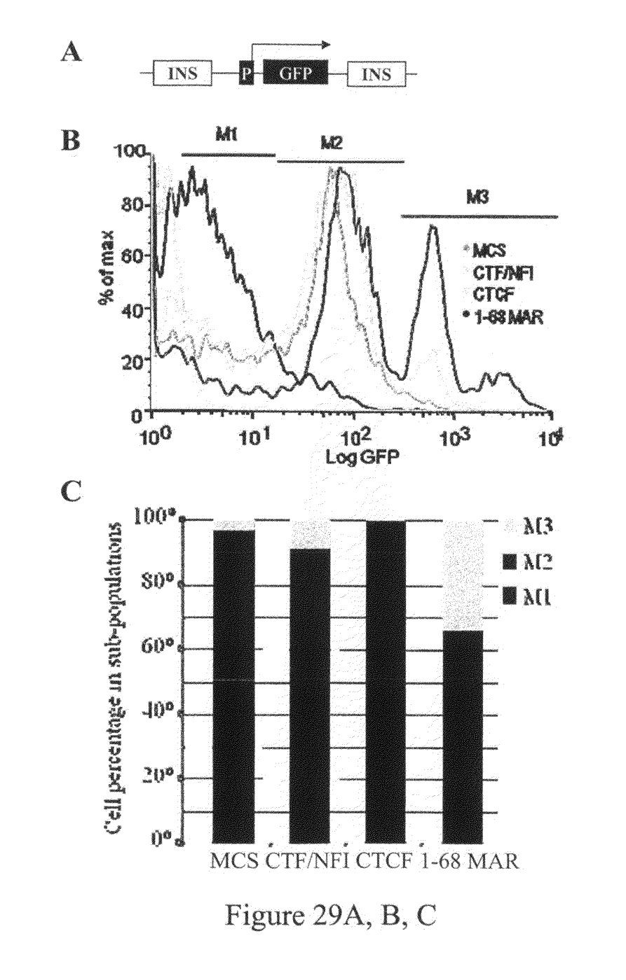

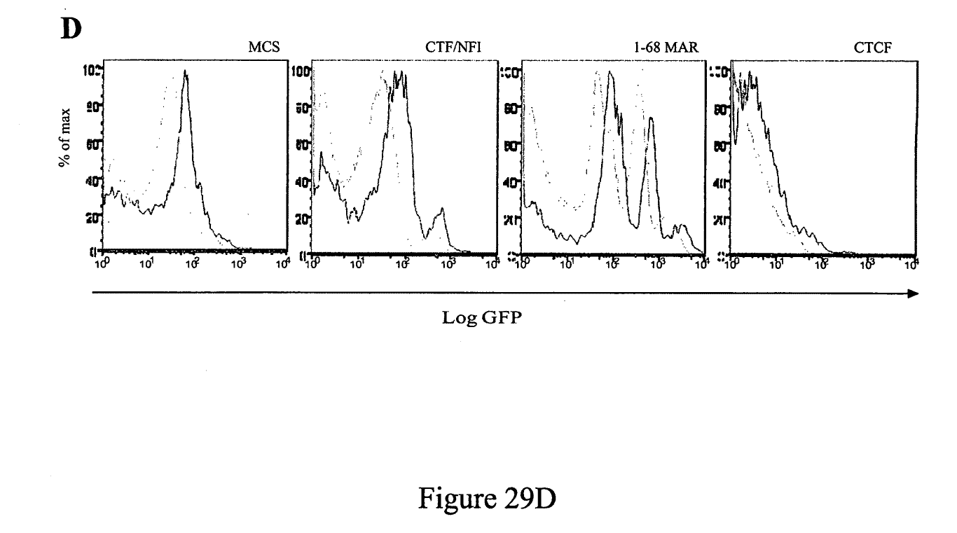

CTF binding sites have been shown to function as barrier elements that can prevent the silencing of telomeric genes (Ferrari et al., 2004; Fourel et al., 2001; Pankiewicz et al., 2005). Nevertheless, whether it may also function as a barrier element upon transgene integration at internal chromosomal loci has not been assessed. The CTF/NFI adeno. (5 bp spacing) or CTCF binding sites were sub-cloned on each side of a SV40 promoter/GFP gene cassette, to address the potential barrier properties of these sequences at random chromosomal locations (FIG. 29A). A multiple cloning site spacer element was cloned in place of the insulators in the negative control plasmid, while the 1-68 matrix attachment region (MAR) element that potently abrogates silencing effects was used as positive control (Girod et al., 2007).

These constructs were stably transfected in HeLa cells, and the GFP fluorescence profile was assessed for each construct on the polyclonal cell population. The distribution of the cell fluorescence in the populations varied from one construct to another but nevertheless yielded reproducible patterns of cells displaying distinct levels of GFP expression. The fluorescence profiles were used to define 3 sub-populations of GFP-positive cells termed M1, M2 and M3, which designate low, medium, and high GFP expression ranges (FIG. 29B). Cells whose fluorescence profile overlapped with the profile of non-transfected cells were considered as non-expressing cells.

In presence of the MAR, most of the cells expressed GFP and their distribution within the population of GFP-positive cells is of 65% in M2 and 35% in M3 (FIG. 29C). This distribution may be explained by a barrier activity of MARs that would shield the transgene from silencing at the site of integration in the host cell chromosomes. In contrast, only about 2% of the GFP-expressing cells are high expressers (M3) for the construct containing a neutral MCS sequence.

An intermediate picture was obtained for the CTF/NF 1 construct when comparing with the MCS and the MAR constructs. Overall, the GFP expression was improved compared to that of cells transfected with the MCS-containing control, with only one third of the cells expressing at low levels (M1), 10% in M2, and a percentile in M3 reaching nearly 10%. The profile from the CTF/NF1 construct was shifted to the right compared to the MCS but to a lesser extent than with the 1-68 MAR (FIG. 29B). These data strongly suggest that CTF/NF1 possesses barrier properties at internal chromosomal positions, and that it should favor transgene expression by limiting silencing effects, in addition to its enhancer-blocking activity.

Populations generated from the MCS, CTF/NF1 and MAR constructs contained around 50%, 60% and 65% of M2 cells, respectively. However, the CTCF construct showed only 10% of cells in the M2 sub-population while the majority of the cells were either expressing at low levels or did not display detectable GFP fluorescence. Thus, flanking a transgene with CTCF binding sites may be deleterious for gene expression, and CTCF may exert a silencing activity, at least in this context.

Applicants next tested whether these elements may act to slow down transgene silencing over time. Applicants performed a time course analysis of GFP expression in stably transfected polyclonal cell pools up to one month (FIG. 29D). The global pattern of expression for each construct appeared to be conserved over time, although a slight shift towards lower fluorescence was generally observed between days 16 and 20. Overall, this indicates that the MAR and CTF/NF1-mediated anti-silencing effects are stable and can withstand cell division.

### 6.4 Insulator-Containing Retroviral Vectors Yield High Titers and Reduced Genotoxicity

Applicants next assessed whether CTCF and CTF/NF1 may shield off the retroviral vector enhancer from activating the expression of cellular genes and/or mediating clonal cell proliferation. The insulators were inserted into the U3 region of both LTRs of the gammaretroviral self-inactivating (SIN) vector SRS.SF.eGFP.pre (Schambach et al., 2006a) (FIG. 30A). Inclusion of CTCF and CTF/NF1 binding sites had little effect on gammaretroviral vector titers, which remained above 10⁷ transducing units per ml, as determined on SC-1 murine fibroblasts. Expression from insulator vectors was slightly slower compared to the control vector, in a range, which is normally seen after introduction of comparable sizes of heterologous sequences into the ΔU3 deletion that is in agreement with observations in an earlier study (Zychlinski et al., 2008). The insulator activity was assessed using the in vitro immortalization (IVIM) assay. The IVIM assay is based on the in vitro selection of insertional mutant clones that gain a proliferative advantage after stable transduction by retroviral vectors. Immortalized mutant clones typically contain a vector insertion within the first intron of the Evi1 gene that results in the insertional upregulation of the Evi1 messenger RNA level. The IVIM Assay measures the replating frequency of mutant clones within the transduced culture (“clonal fitness”) as well as the incidence of mutation events between different transduced cultures, because not every culture may produce a replating clone. For the testing of the newly developed insulators we chose a SIN retroviral vector that contains the strong viral SFFV enhancer/promoter as internal promoter and was previously shown to be transforming in every culture tested (incidence of 2×10-5), with a replating frequency/copy number of ˜0.0035 (mean of n=10). Both CTCF and CTF/NF1 insulators were able to reduce the replating fequency/copy number by 4 and 5-fold, respectively, when compared to the uninsulated vector (SRS.SF. CTCF versus SRS.SF. p=0.055; SRS.SF. CTF/NF1 versus SRS.SF. p=0.043; n=7 each, Wilcoxon two sample test, FIG. 30B). The lower replating frequency was paralleled by lower Evi1 expression levels in presence of the insulated vectors within the mass cultures before replating (FIG. 30C).

### 6.5 Discussion

Designing new generations of gene transfer viral vectors is a promising avenue to achieve safer gene therapy. The implementation of genetic insulator elements in retroviral vectors is intended to allow the transgene cassette to behave as an autonomously regulated expression unit once integrated in the host cell genome. When flanking the transgene cassette, insulators may be beneficial in two ways: i) enhancer-blockers would limit the range of action of the viral vector enhancer on nearby cellular genes, thus decreasing the risk of insertional activation of cellular genes, ii) barrier elements would stop the spreading of silent chromatin, to ensure long-term transgene expression and counteract position effect (Gaszner and Felsenfeld, 2006).

This study describes the design of a standardized screening procedure to assess the enhancer-blocking activity of insulator elements. Unlike approaches based on the assay of mRNA levels, secretion of a reporter protein or antibiotic resistance, this assay can be used to process quickly large cell populations to provide a quantitative estimation of the insulating activity with a single-cell resolution. This complements a recently described quantitative assay of the barrier function of insulators specifically integrated at mammalian cell telomeres (Esnault et al., 2009).

Use of this screening procedure allowed the identification and assay of a collection of novel insulating sequences comprising optimized binding sites for different types of insulator proteins. For instance, a 472 bp element comprising 12 CTCF binding sites was able to reconstitute the enhancer blocking activity mediated by the full-length 1.2 kb cHS4, whereas one copy of the cHS4 core showed little activity in these conditions. These findings are consistent with recent studies showing that a single copy of the cHS4 250 bp core does not recapitulate the insulating function of the full-length element (Arumugam et al., 2009). Binding sites for the CTF/NF1 transcription factors family were also shown to have significant enhancer blocking activity, even from a single binding site. The insulator activity could be fully attributed to CTF/NF1 proteins upon knock-down assays, thus establishing a previously unknown enhancer-blocking activity for this family of transcriptional regulators. The compatibility of the insulator size with retroviral vectors had to be considered, as the insertion of long DNA elements in the 3′LTR has been directly linked to reduced vector titers and impairment in the transduction efficiency (Nielsen et al., 2009; Urbinati et al., 2009). Therefore, insulator elements of varying size were designed, so as to fit the LTR of retro and/or lentiviral vectors without affecting negatively viral vector preparation or transgene expression. We find that the insulator potency correlates overall well with insulator length, but that it can be clearly distinguished from simple distance effects, as mediated by the interposition of non-specific spacer DNA sequences between the enhancer and the promoter. Nevertheless, we find that elements as short as 20 bp can still mediate significant enhancer-blocking function.

Derivatives of CTF/NF1 and CTCF-binding insulator sequences yielded reduced genotoxicity when inserted in gammaretroviral self-inactivating vectors without altering titers significantly. These results are promising, as insulators able to block 50% of the activation mediated by a strong LTR enhancer led to a 4 to 5-fold reduction of the retroviral vector genotoxicity in an in vitro immortalization assay. Furthermore, the decreased occurrence of clonal cell proliferation correlated well with the 5 to 10-fold lower expression levels of Evi1 message noted in presence of the CTF/NF1 insulator. This implies that the enhancer-blocking activity detected with the plasmid-based assay was preserved in the context of the viral vector LTR. The barrier activity of the novel insulating elements was also assessed in the context of random transgene chromosomal integration upon stable transfection. Surprisingly, flanking the transgene with CTCF binding sites led to a decrease, in expression that was stably propagated upon cell population growth. Prior studies on the cHS4 insulator had shown that deletion of the CTCF binding sites were associated with a loss of the enhancer blocking activity but that it did not alter the barrier function of the element (Bell et al., 1999; Burgess-Beusse et al., 2002; Chung et al., 1997). However, prior work on the natural cHS4 locus could not easily assess a potential silencing effect of CTCF in addition to its enhancer-blocking activity. Extensive investigations on the functions of CTCF in various genomic contexts led to the conclusion that it is a ubiquitous key-player in genome-wide organization of the chromatin architecture. Besides its insulator activity, it has also been implicated in imprinting and in either the repression or the activation of transcription. Even though current prevailing models of CTCF action rely on a looping mechanism, CTCF might also orchestrate genome architecture through epigenetic chromatin modifications such as the recruitment of chromatin modifying proteins able to locally alter the chromatin structure (Phillips and Corces, 2009; Zlatanova and Caiafa, 2009a). Overall, the multiplicity of functions of CTCF suggests that its mode of action may depend on the biological context (Zlatanova and Caiafa, 2009b). As such, implementing CTCF-binding synthetic sequences in vectors that integrate at multiple and relatively random loci in the cell genome, a mediated by viral transduction, may yield effects that may not be fully predicted from CTCF mode of action at the cHS4 or at imprinted loci (D'Apolito et al., 2009). Large scale analysis of the effect of insulated and non-insulated vectors will be required to address these issues.

Transgenes flanked with binding sites for CTF/NF 1 appeared to be protected from silencing effects when integrated at random internal chromosomal loci. This observation is consistent with previous studies that demonstrated a role for CTF/NF I proteins as barrier elements that can block the propagation of silent chromatin structures, and thus protect transgenes from silencing effects (Esnault et al., 2009; Ferrari et al., 2004; Fourel et al., 2001; Pankiewicz et al., 2005). As such, the CTF/NF1 insulator appears to act both as enhancer-blocker and as a barrier insulator element. Thus, CTF/NF1 binding sites may be able to maintain a euchromatic status of the provirus, which may contribute favorably to the stable production of retroviral vectors. This finding may also be of advantagious for the perspective of using tissue specific promoters to drive transgene expression, which may be potentially weaker than strong ubiquitous promoters of viral origin, thus reducing the likelihood of the silencing of the therapeutic gene over time. Finally, the tropism of retroviral vectors for specific genomic regions such as certain proto-oncogenes still remains a major issue for gene therapy safety (Metais and Dunbar, 2008; Modlich et al., 2008), despite many recent progress (Cassani et al., 2009). Thus, it would be advantageous to develop targeted integration strategies of insulated or non-insulated vectors. However, this remains difficult at present. Counteracting position effects and the occurrence of poor expression of some integrated vectors with insulators may allow favorable therapeutic outcome from lower multiplicities of infections and reduced vector integration events (Urbinati et al., 2009), which should further reduce the risk of both activating and inactivating integration events.

### Example 7

### FIG. 26: Schematic Diagrams of Insulator/Enhancer-Blocker Assay Systems and Reporter Genes Expression Analysis

(A) Schematic representation of the vectors used for the insulator assay based on the quantitation of neomycin-resistant colonies. A reporter gene (neo) conferring resistance to the neomycin (G418) antibiotic is driven by the y-globin promoter under the control of either the β-globin LCR element or the FrMu-LV LTR-containing enhancer (in both orientations). The level of expression of that reporter gene is assessed by the number of neomycin-resistant colonies obtained after stable transfections. The insulated neo gene is flanked by two copies of the 1.2 kb cHS4 insulator (interposed and external positions, referred to as int. and ext., respectively), while its non-insulated counterpart is flanked by just one cHS4 copy at the external position.

(B) Schematic illustration of the quantitative assay for enhancer-blockers. Constructs are composed of a BFP gene under the control of the promoter and enhancer-containing FrMu-LV LTR, and a GFP gene under the control of the minimal CMV promoter. The insulated GFP gene is flanked by two copies of the 1.2 kb cHS4 while the BFP gene serves as an internal reference for transfection efficacy and transgene expression level in each analyzed cell. Without an insulator at the int. position, the FrMu-LV LTR enhancer is driving expression of both reporter genes. The interposed copy of the cHS4 (int.) has been substituted by the 250 bp cHS4 core or by DNA spacers of various lengths.

(C) Percentage of neomycin-resistant colonies counted 2 to 3 weeks after transfection and G418 selection of HeLa (dashed bars) and K562 (black bars) cells. The presence of an enhancer (Enh.) and/or of an interposed cHS4 insulator are indicated as depicted in panel A. Transfected constructs contained as an enhancer either the β-globin LCR element (LCR) or the FrMu-LV LTR in one orientation (LTR) or in the inverted orientation (LTRinv). The percentile of resistant colonies obtained in the absence of the interposed copy of the cHS4 was set to 100%.

(D) Cytofluorometric analysis of the cHS4 insulator activity using the quantitative assay in transiently transfected HeLa cells. The three panels show data of the same two representative cell populations obtained 48 hours after transfection: cell populations transfected with the assay construct containing the interposed (int.) copy of the cHS4 (as described in panel B) are shown in blue, while profiles obtained with constructs without an interposed cHS4 are depicted in red. From left to right, panels present respectively the GFP expression of BFP positive cells, the BFP expression of total cell population, and the fluorescence levels of BFP as a function of GFP for the total cell population. Black profiles correspond to non-transfected cells control.

(E) Quantitative analysis of the cHS4 insulator enhancer-blocking activity. GFP fluorescence values were determined 48 hours after HeLa cells transfection and were normalized to those of BFP for each analyzed cell. Averages of the normalized fluorescence are plotted for cell populations transfected with constructs containing the indicated insulator as described in panel B, while Δ ins. refers to the construct without any interposed insulator sequence. Data were normalized to the values obtained with a construct lacking both copies of the insulator. Elements interposed between the enhancer and the promoter driving GFP expression, as well as their respective size are indicated. Spacers refer to portions of coding sequences of designated sizes. The p value was determined by a two-tailed t-test.

### FIG. 27: Quantitative Analysis of Synthetic CTCF and CTF/NFI Binding Sites Enhancer-Blocking Activity Compared to the cHS4

(A) Sequence description and pairwise alignment of the different types of CTCF and CTF/NFI binding sites constructed and assessed. Conserved nucleotides between two sequences are highlighted in red and a star marks their position.

(B) (C) Quantitative analysis of the enhancer-blocking activity of the CTCF and CTF/NFI binding sites. Hela cells transfections, determination of GFP to BFP fluorescence ratio and normalization to the values obtained without any insulator are as described in the legend to FIG. 26E. The numbers of repeats are indicated for each designated binding site, and the spacing between to adjacent CTF/NFI binding sites is specified (5 or 10 bp).

(D) Quantitative analysis of the enhancer-blocking activity of CTCF and CTF/NFI binding sites in stable transfections. The mean GFP expression normalized to BFP expression per cell is plotted for each HeLa cell population 2 to 3 weeks after the antibiotic selection of cells transfected with the constructs depicted in FIG. 26B. Data were normalized to the values obtained with construct lacking both insulator copies. Elements interposed between the enhancer and the promoter of the GFP are indicated on the Y-axis. p values of two-tailed t-tests are indicated.

### FIG. 28: CTF/NFI Proteins Mediate the Enhancer-Blocking Activity of Cognate DNA Binding Sites

(A) Quantitative analysis of the enhancer-blocking properties of CTF/NFI binding sites enhancer-blocking properties in comparison with a 250 bp DNA spacer upon siRNA-mediated knocking-down of CTF/NFI expression. HeLa cells were transfected with siRNA targeting CTF/NF1 (controls: mock transfection or scrambled siRNA) and subsequently transfected with the insulator assay constructs containing either a neutral spacer of 250 bp or 7 binding sites for CTF/NF1 (Adeno, 5 bp spacing.). FACS analyses were performed 48 hours after DNA transfection of HeLa cells. The average of the GFP to BFP fluorescence ratio was determined and plotted as described in the legend of FIG. 26E. The fluorescence ratios were normalized to those obtained from the mock transfection of the siRNA and the transfection of the DNA construct containing the 250 bp spacer. The p value of two-tailed t-test is indicated.

(B) Western-blot analysis of the cell populations analyzed in panel A. The immunoblot was performed on same day as FACS analysis. GAPDH was used as a loading control.

### FIG. 29: CTF/NFI Binding Sites Dampen Chromosomal Position-Effect

(A) Schematic representation of the insulated GFP transgene. GFP expression was driven by a SV40 promoter and the effect of elements inserted on both sides of the transgene was evaluated in stable transfections of HeLa cells.

(B) Results of representative FACS analysis for GFP expression of HeLa cell populations stably transfected with constructs described in panel A (16 days after transfection). The GFP transgene was flanked by either a multiple cloning site (MCS), or 7 binding sites for CTF/NFI (Adeno., 5 bp spacing), or one copy of the 1-68 MAR element. The profile of non-transfected cells is depicted in grey. The population of GFP-positive cells, i.e. the total cell population excluding non-expressing cells, was divided in 3 sub-populations as following: M1 designates cells expressing low levels GFP, while M2 and M3 designate cells with medium or high ranges of GFP levels respectively.

(C) Relative distribution of each sub-population of cells according to GFP expression levels. M1, 2 and 3 sub-populations are defined as described in panel B. Results are expressed in percentage of cells in the designated sub-population relatively to the population of GFP-positive cells (excluding non-expressing cells).

(D) Time course FACS analysis of the GFP transgene expression when flanked with various insulators in stably transfected HeLa cells. Results of FACS analysis were acquired after 16, 20, 27 and 30 days of antibiotic selection post-transfection.

### FIG. 30: CTF/NFI and CTCF Binding Sites Decrease the Genotoxicity or Retroviral Vectors

(A) Vector architecture of the gammaretroviral self-inactivating (SIN) vector SRS.SF.eGFP.pre shown as provirus. It contains a splice-competent leader region and posttranscriptional regulatory element (PRE) of the woodchuck hepatitis virus. The U3 region is almost completely deleted, leaving only the integrase attachment sites intact. eGFP is driven by the enhancer/promoter elements derived from spleen focus-forming virus SF enhancer/promoter. In the insulated vectors the insulator sequences were inserted into the U3 region of the vector's LTRs.

(B) The introduction of the insulator sequences into the LTRs of the SRS.SF.eGFP.pre vectors reduced its transformation potential. The replating frequencies of Lin⁻ cells corrected to the mean copy number as measured in the DNA of mass cultures were plotted for insulated vectors and for the parental uninsulated SRS.SF gammaretroviral vector. Insulators implemented in retroviral vectors are 6 copies of CTCF binding sites and 7 copies of CTF/NF1 binding sites (Adeno, 5 bp spacing). The replating frequency/copy number of the SRS.SF.eGFP.pre.CTCF vector was ˜4 fold reduced and the SRS.SF.eGFP.pre.CTF/NF1˜5 fold. The data points shown for the SRS.SF.eGFP.pre vector contains those generated in this study (black dots) and previously published data (grey dots, Modlich et al., 2009). The horizontal lines indicate the respective medians of the populations.

(C) Quantitative real-time PCR analysis of Evi1 expression levels in the mass cultures of vector transduced lineage negative BM cells at the day of replating. Evi1 expression was lower in cultures transduced with insulated vectors compared to non-insulated control (SRS.SF.eGFP.pre). Evi1 expression in expanded and untransduced mock cells was set to 1.

### Example 8

### 8.1 Consensus CTCF Binding Sites

Consensus CTCF binding sites correspond to direct repeats of the consensus binding motif (Kim et al., 2007) and separated from one another with spacers up to the size of a native binding site (40 bp).

SEQ ID N^{o} 12
Gcgatgccgccccctggtggccagtaatcgcaaggctaagtaatcact
gccccctggtggccgccagtctgatacgcgttttacaaccgccccctg
gtggccgtgggagacatctagtgcacgagagtgccccctggtggccaa
accgtagcctaggcatattgtactgccccctggtggccggcaatatgg
ctagcgatgactcggcgccccctggtggccactacgttctagtg

### 8.2 Consensus CTCF Binding Sites: (12 Copies) SEQ ID No 11

gcgatgccgccccctggtggccagtaatcgcaaggctaagtaatcact
gccccctggtggccgccagtctgatacgcgttttacaaccgccccctg
gtggccgtgggagacatctagtgcacgagagtgccccctggtggccaa
accgtagcctaggcatattgtactgccccctggtggccggcaatatgg
ctagcgatgactcggcgccccctggtggccactacgttctagtggcga
tgccgccccctggtggccagtaatcgcaaggctaagtaatcactgccc
cctggtggccgccagtctgatacgcgttttacaaccgccccctggtgg
ccgtgggagacatctagtgcacgagagtgccccctggtggccaaaccg
tagcctaggcatattgtactgccccctggtggccggcaatatggctag
cgatgactcggcgccccctggtggccactacgttctagtg
(472 bp)

### 8.3 CTF/NF1 Binding Sites from the Adenovirus type II Origin of Replication:

Adeno. CTF/NF1 binding sites are composed of direct repeats of the CTF/NF1 binding site from the adenovirus type II origin of replication isolated from the pNF7CAT plasmid (Tarapore et al., 1997).
1 CTF/NF1 binding site: SEQ ID No 1
   ttggcaacgtgccataagca (20 bp)
1 CTF/NF1 binding site, 5 bp flanking: SEQ ID No 13
   actagttggc aacgtgccat aagc (24 bp)
1 CTF/NF1 binding site, 5 bp spacing: SEQ ID No 24
   attggcaacgtgccataagc (20 bp)
3 CTF/NF1 binding sites, 5 bp spacing: SEQ ID No 25
   taagcttgcattggcaacgtgccataagcattggcaacgtgccataagcattggcaacgtgccataagcgaattgggggat
3 CTF/NF1 binding sites, 5 bp spacing (reverse strand): SEQ ID No 26
   atcccccaattcgcttatggcacgttgccaatgcttatggcacgttgccaatgcttatggcacgttgccaatgcaagctta
4 CTF/NF1 binding sites, 5 bp spacing (reverse strand): SEQ ID No 27
   atcccccaattcgcttatggcacgttgccaatgcttatggcacgttgccaatgcttatggcacgttgccaatgcttatggcacgttgccaat gcaagctta
7 CTF/NF1 binding sites, 5 bp spacing: SEQ ID No 14
   atcgataagcttgcattggcaacgtgccataagcattggcaacgtgccataagcattggcaacgtgccataagcattggcaacgtgcca taagcattggcaacgtgccataagcattggcaacgtgccataagcattggcaacgtgccataagcggggggatcc
3 CTF/NF1 binding sites, 10 bp spacing: SEQ ID No 15
   ctagattggcaatctgccatgctagcttgtttggcagactgccatcctaggtcagttggcatgatgccat
4 CTF/NF1 binding sites, 10 bp spacing: SEQ ID No 28
   atgtcattggcaaactgccattgcatctgtattggcagtatgccatgttactcttgttggcactgtgccatgatacagatattggcaccttgcc atctag
7 CTF/NF1 binding sites, 10 bp spacing: SEQ ID No 16
   ctagattggcaatctgccatgctagcttgtaggcagactgccatactaggtcagttggcatgatgccatctagatgtcattggcaaactgc cattgcatctgtattggcagtatgccatgttactcttgttggcactgtgccatgatacagatattggcaccttgccat

### 8.4 Consensus CTF/NF1 Binding Sites

The consensus CTF/NF1 binding site was obtained from SELEX-SAGE experiments (Roulet et al., 2002). SEQ ID No 29
   ttggcNNNNNgccaa
3 CTF/NF1 binding sites, 5 bp spacing: SEQ ID No 17.
   ctagattggcaatctgccaagctgtttggcagactgccaacccagttggcatgatgccaa
4 CTF/NF1 binding sites, 5 bp spacing: SEQ ID No 23
   actagattggcaaactgccaatggtattggcagtatgccaagtttgttggcactgtgccaagaatattggcaccttgcca
7 CTF/NF1 binding sites, 5 bp spacing: SEQ ID No 18
   ctagattggcaatctgccaagctgtttggcagactgccaacccagttggcatgatgccaactagattggcaaactgccaatggtattggc agtatgccaagtttgttggcactgtgccaagaatattggcaccttgccaa
3 CTF/NF1 binding sites, 10 bp spacing: SEQ ID No 19
   ctagattggcaatctgccaagctagcttgtttggcagactgccaacctaggtcagttggcatgatgccaa
4 CTF/NF1 binding sites, 10 bp spacing: SEQ ID No 22
   actagatgtcattggcaaactgccaatgcatctgtattggcagtatgccaagttactcttgttggcactgtgccaagatacagatattggca ccttgccaa
7 CTF/NF1 binding sites, 10 bp spacing: SEQ ID No 20
   ctagattggcaatctgccaagctagcttgtttggcagactgccaacctaggtcagttggcatgatgccaactagatgtcattggcaaactg ccaatgcatctgtattggcagtatgccaagttactcttgttggcactgtgccaagatacagatattggcaccttgccaa

### 8.5 CTCF Consensus Sequence: SEQ ID No 21

gccccctggtggcc

### 8.6 CTCF Consensus Sequence (Complementary): SEQ ID No 30

ggccaccagg gggc

### 8.7 CTCF Binding Sites

SEQ ID No 2
   cccagggatg taattacgtc cctcccccgc tagggggcag ca
SEQ ID No 3
   cccaggcctg cactgccgcc tgccggcagg ggtccagtc

### REFERENCES

Aker, M., Tubb, J., Groth, A. C., Bukovsky, A. A., Bell, A. C., Felsenfeld, G., Kiem, H. P., Stamatoyannopoulos, G., and Emery, D. W. (2007). Extended core sequences from the cHS4 insulator are necessary for protecting retroviral vectors from silencing position effects. Hum Gene Ther 18, 333-343.
Alevizopoulos, A., and Mermod, N. (1996). Antagonistic regulation of a proline-rich transcription factor by transforming growth factor beta and tumor necrosis factor alpha. J Biol Chem 271, 29672-29681.
Alevizopoulos, A., Y. Dusserre, M. Tsai-Pflugfelder, T. von der Weid, W. Wahli, and N. Mermod. 1995. A proline-rich TGF-beta-responsive transcriptional activator interacts with histone H3. Genes Dev 9:3051-66.
Baum, C., Dullmann, J., Li, Z., Fehse, B., Meyer, J., Williams, D. A., and von Kalle, C. (2003). Side effects of retroviral gene transfer into hematopoietic stem cells. Blood 101, 2099-2114.
Baum, C., Richters, A., and Ostertag, W. (1999). Retroviral vector-mediated gene expression in hematopoietic cells. Curr Opin Mol Ther 1, 605-612.
Baum, C., von Kalle, C., Staal, F. J., Li, Z., Fehse, B., Schmidt, M., Weerkamp, F., Karlsson, S., Wagemaker, G., and Williams, D. A. (2004). Chance or necessity? Insertional mutagenesis in gene therapy and its consequences. Mol Ther 9, 5-13.
Baur, J. A., Y. Zou, J. W. Shay, and W. E. Wright. 2001. Telomere position effect in human cells. Science 292:2075-7.
Bell, A. C., A. G. West, and G. Felsenfeld. 2001. Insulators and boundaries: versatile regulatory elements in the eukaryotic. Science 291:447-50.
Bell, A. C., West, A. G., and Felsenfeld, G. (1999). The protein CTCF is required for the enhancer blocking activity of vertebrate insulators. Cell 98, 387-396.
Blasco, M. A. 2007. The epigenetic regulation of mammalian telomeres. Nat Rev Genet 8:299-309.
Burgess-Beusse, B., Farrell, C., Gaszner, M., Litt, M., Mutskov, V., Recillas-Targa, F., Simpson, M., West, A., and Felsenfeld, G. (2002). The insulation of genes from external enhancers and silencing chromatin. Proc Natl Acad Sci USA 99 Suppl 4, 16433-16437.
Bushman, F. D. (2003). Targeting survival: integration site selection by retroviruses and LTR-retrotransposons. Cell 115, 135-138.
Cassani, B., Montini, E., Maruggi, G., Ambrosi, A., Mirolo, M., Selleri, S., Biral, E., Frugnoli, I., Hernandez-Trujillo, V., Di Serio, C., et al. (2009). Integration of retroviral vectors induces minor changes in the transcriptional activity of T cells from ADA-SCID patients treated with gene therapy. Blood.
Cavazzana-Calvo, M., Hacein-Bey, S., de Saint Basile, G., Gross, F., Yvon, E., Nusbaum, P., Selz, F., Hue, C., Certain, S., Casanova, J. L., et al. (2000). Gene therapy of human severe combined immunodeficiency (SCID)-X1 disease. Science 288, 669-672.
Chalmers, D., Ferrand, C., Apperley, J. F., Melo, J. V., Ebeling, S., Newton, I., Duperrier, A., Hagenbeek, A., Garrett, E., Tiberghien, P., and Garin, M. (2001). Elimination of the truncated message from the herpes simplex virus thymidine kinase suicide gene. Mol Ther 4, 146-148.
Chung, J. H., Bell, A. C., and Felsenfeld, G. (1997). Characterization of the chicken beta-globin insulator. Proc Natl Acad Sci USA 94, 575-580.
Chung, J. H., Whiteley, M., and Felsenfeld, G. (1993). A 5′ element of the chicken beta-globin domain serves as an insulator in human erythroid cells and protects against position effect in Drosophila. Cell 74, 505-514.
Cloos, P. A., J. Christensen, K. Agger, A. Maiolica, J. Rappsilber, T. Antal, K. H. Hansen, and K. Helin. 2006. The putative oncogene GASC1 demethylates tri- and dimethylated lysine 9 on histone H3. Nature 442:307-11.
Cohen, R. B., Sheffery, M., and Kim, C. G. (1986). Partial purification of a nuclear protein that binds to the CCAAT box of the mouse alpha 1-globin gene. Mol Cell Biol 6, 821-832.
Cohen-Haguenauer, O., Restrepo, L. M., Masset, M., Bayer, J., Dal Cortivo, L., Marolleau, J. P., Benbunan, M., Boiron, M., and Marty, M. (1998). Efficient transduction of hemopoietic CD34+ progenitors of human origin using an original retroviral vector derived from Fr-MuLV-FB29: in vitro assessment. Hum Gene Ther 9, 207-216.
Defossez, P. A., and Gilson, E. (2002). The vertebrate protein CTCF functions as an insulator in Saccharomyces cerevisiae. Nucleic Acids Res 30, 5136-5141.
Dhillon, N., and R. T. Kamakaka. 2000. A histone variant, Htz1p, and a Sir1p-like protein, Esc2p, mediate silencing at HMR. Mol Cell 6:769-80.
Eckert, H. G., Stockschlader, M., Just, U., Hegewisch-Becker, S., Grez, M., Uhde, A., Zander, A., Ostertag, W., and Baum, C. (1996). High-dose multidrug resistance in primary human hematopoietic progenitor cells transduced with optimized retroviral vectors. Blood 88, 3407-3415.
Emery, D. W., Yannaki, E., Tubb, J., and Stamatoyannopoulos, G. (2000). A chromatin insulator protects retrovirus vectors from chromosomal position effects. Proc Natl Acad Sci USA 97, 9150-9155.
Emery, D. W., Yannaki, E., Tubb, J., Nishino, T., Li, Q., and Stamatoyannopoulos, G. (2002). Development of virus vectors for gene therapy of beta chain hemoglobinopathies: flanking with a chromatin insulator reduces gamma-globin gene silencing in vivo. Blood 100, 2012-2019.
Esnault, G., Majocchi, S., Martinet, D., Besuchet-Schmutz, N., Beckmann, J. S., and Mermod, N. (2009). Transcription factor CTF1 acts as a chromatin domain boundary that shields human telomeric genes from silencing. Mol Cell Biol.
Fan, J. Y., D. Rangasamy, K. Luger, and D. J. Tremethick. 2004. H2A.Z alters the nucleosome surface to promote HP1 alpha-mediated chromatin fiber folding. Mol Cell 16:655-61.
Ferrari, S., K. C. Simmen, Y. Dusserre, K. Muller, G. Fourel, E. Gilson, and N. Mermod. 2004. Chromatin domain boundaries delimited by a histone-binding protein in yeast. J Biol Chem 279:55520-30.
Flahaut, M., A. Muhlethaler-Mottet, D. Martinet, S. Fattet, K. B. Bourloud, K. Auderset, R. Meier, N. B. Schmutz, O. Delattre, J. M. Joseph, and N. Gross. 2006. Molecular cytogenetic characterization of doxorubicin-resistant neuroblastoma cell lines: evidence that acquired multidrug resistance results from a unique large amplification of the 7q21 region. Genes Chromosomes Cancer 45:495-508.
Fourel, G., C. Boscheron, E. Revardel, E. Lebrun, Y. F. Hu, K. C. Simmen, K. Muller, R. Li, N. Mermod, and E. Gilson. 2001. An activation-independent role of transcription factors in insulator function. EMBO Rep 2:124-32.
Fourel, G., Magdinier, F., and Gilson, E. (2004). Insulator dynamics and the setting of chromatin domains. Bioessays 26, 523-532.
Garcia-Cao, M., R. O'Sullivan, A. H. Peters, T. Jenuwein, and M. A. Blasco. 2004. Epigenetic regulation of telomere length in mammalian cells by the Suv39h1 and Suv39h2 histone methyltransferases. Nat Genet 36:94-9.
Gaszner, M., and Felsenfeld, G. (2006). Insulators: exploiting transcriptional and epigenetic mechanisms. Nat Rev Genet 7, 703-713.
Girod, P. A., D. Q. Nguyen, D. Calabrese, S. Puttini, M. Grandjean, D. Martinet, A. Regamey, D. Saugy, J. S. Beckmann, P. Bucher, and N. Mermod. 2007. Genome-wide prediction of matrix attachment regions that increase gene expression in mammalian cells. Nat Methods 4:747-53.
Hacein-Bey-Abina, S., Garrigue, A., Wang, G. P., Soulier, J., Lim, A., Morillon, E., Clappier, E., Caccavelli, L., Delabesse, E., Beldjord, K., et al. (2008). Insertional oncogenesis in 4 patients after retrovirus-mediated gene therapy of SCID-X1. J Clin Invest 118, 3132-3142.
Hacein-Bey-Abina, S., Le Deist, F., Carlier, F., Bouneaud, C., Hue, C., De Villartay, J. P., Thrasher, A. J., Wulffraat, N., Sorensen, R., Dupuis-Girod, S., et al. (2002). Sustained correction of X-linked severe combined immunodeficiency by ex vivo gene therapy. N Engl J Med 346, 1185-1193.
Hacein-Bey-Abina, S., Von Kalle, C., Schmidt, M., McCormack, M. P., Wulffraat, N., Leboulch, P., Lim, A., Osborne, C. S., Pawliuk, R., Morillon, E., et al. (2003). LMO2-associated clonal T cell proliferation in two patients after gene therapy for SCID-X1. Science 302, 415-419.
Harraghy, N., Gaussin, A., and Mermod, N. (2008). Sustained transgene expression using MAR elements. Curr Gene Ther 8, 353-366.
Hino, S., Akasaka, K., and Matsuoka, M. (2006). Sea urchin arylsulfatase insulator exerts its anti-silencing effect without interacting with the nuclear matrix. J Mol Biol 357, 18-27.
Hockemeyer, D., J. P. Daniels, H. Takai, and T. de Lange. 2006. Recent expansion of the telomeric complex in rodents: Two distinct POT1 proteins protect mouse telomeres. Cell 126:63-77.
Hoppe, G. J., J. C. Tanny, A. D. Rudner, S. A. Gerber, S. Danaie, S. P. Gygi, and D. Moazed. 2002. Steps in assembly of silent chromatin in yeast: Sir3-independent binding of a Sir2/Sir4 complex to silencers and role for Sir2-dependent deacetylation. Mol Cell Biol 22:4167-80.
Ishii, K., G. Arib, C. Lin, G. Van Houwe, and U. K. Laemmli. 2002. Chromatin boundaries in budding yeast: the nuclear pore connection. Cell 109:551-62.
Ito, T., T. Ikehara, T. Nakagawa, W. L. Kraus, and M. Muramatsu. 2000. p300-mediated acetylation facilitates the transfer of histone H2A-H2B dimers from nucleosomes to a histone chaperone. Genes Dev 14:1899-907.
Jones, K. A., Yamamoto, K. R., and Tjian, R. (1985). Two distinct transcription factors bind to the HSV thymidine kinase promoter in vitro. Cell 42, 559-572.
Kalman, L., Lindegren, M. L., Kobrynski, L., Vogt, R., Hannon, H., Howard, J. T., and Buckley, R. (2004). Mutations in genes required for T-cell development: IL7R, CD45, IL2RG, JAK3, RAG1, RAG2, ARTEMIS, and ADA and severe combined immunodeficiency: HuGE review. Genet Med 6, 16-26.
Kelleher, C., I. Kurth, and J. Lingner. 2005. Human protection of telomeres 1 (POT1) is a negative regulator of telomerase activity in vitro. Mol Cell Biol 25:808-18.
Kellum, R., and Schedl, P. (1992). A group of scs elements function as domain boundaries in an enhancer-blocking assay. Mol Cell Biol 12, 2424-2431.
Kim, T. H., Abdullaev, Z. K., Smith, A. D., Ching, K. A., Loukinov, D. I., Green, R. D., Zhang, M. Q., Lobanenkov, V. V., and Ren, B. (2007). Analysis of the vertebrate insulator protein CTCF-binding sites in the human genome. Cell 128, 1231-1245.
Klose, R. J., K. Yamane, Y. Bae, D. Zhang, H. Erdjument-Bromage, P. Tempst, J. Wong, and Y. Zhang. 2006. The transcriptional repressor JHDM3A demethylates trimethyl histone H3 lysine 9 and lysine 36. Nature 442:312-6.
Kourmouli, N., P. Jeppesen, S. Mahadevhaiah, P. Burgoyne, R. Wu, D. M. Gilbert, S. Bongiomi, G. Prantera, L. Fanti, S. Pimpinelli, W. Shi, R. Fundele, and P. B. Singh. 2004. Heterochromatin and tri-methylated lysine 20 of histone H4 in animals. J Cell Sci 117:2491-501.
Kruse, U., Qian, F., and Sippel, A. E. (1991). Identification of a fourth nuclear factor I gene in chicken by cDNA cloning: NFI-X. Nucleic Acids Res 19, 6641.
Kurdistani, S. K., and M. Grunstein. 2003. Histone acetylation and deacetylation in yeast. Nat Rev Mol Cell Biol 4:276-84.
Kutler, D. I., Singh, B., Satagopan, J., Batish, S. D., Berwick, M., Giampietro, P. F., Hanenberg, H., and Auerbach, A. D. (2003). A 20-year perspective on the International Fanconi Anemia Registry (IFAR). Blood 101, 1249-1256.

Laufs, S., Nagy, K. Z., Giordano, F. A., Hotz-Wagenblatt, A., Zeller, W. J., and Fruehauf, S. (2004). Insertion of retroviral vectors in NOD/SCID repopulating human peripheral blood progenitor cells occurs preferentially in the vicinity of transcription start regions and in introns. Mol Ther 10, 874-881.
Leonard, W. J., and O'Shea, J. J. (1998). Jaks and STATs: biological implications. Annu Rev Immunol 16, 293-322.
Li, B., S. G. Pattenden, D. Lee, J. Gutierrez, J. Chen, C. Seidel, J. Gerton, and J. L. Workman. 2005. Preferential occupancy of histone variant H2AZ at inactive promoters influences local histone modifications and chromatin remodeling. Proc Natl Acad Sci USA 102:18385-90.
Li, C. L., Xiong, D., Stamatoyannopoulos, G., and Emery, D. W. (2009). Genomic and functional assays demonstrate reduced gammaretroviral vector genotoxicity associated with use of the cHS4 chromatin insulator. Mol Ther 17, 716-724.
Li, Z., Dullmann, J., Schiedlmeier, B., Schmidt, M., von Kalle, C., Meyer, J., Forster, M., Stocking, C., Wahlers, A., Frank, O., et al. (2002). Murine leukemia induced by retroviral gene marking. Science 296, 497.
Li, Z., Schwieger, M., Lange, C., Kraunus, J., Sun, H., van den Akker, E., Modlich, U., Serinsoz, E., Will, E., von Laer, D., et al. (2003). Predictable and efficient retroviral gene transfer into murine bone marrow repopulating cells using a defined vector dose. Exp Hematol 31, 1206-1214.
Lin, S., D. Lin, and A. D. Riggs. 1976. Histones bind more tightly to bromodeoxyuridine-substituted DNA than to normal DNA. Nucleic Acids Res 3:2183-91.
Liu, J. M., Kim, S., Read, E. J., Futaki, M., Dokal, I., Carter, C. S., Leitman, S. F., Pensiero, M., Young, N. S., and Walsh, C. E. (1999). Engraftment of hematopoietic progenitor cells transduced with the Fanconi anemia group C gene (FANCC). Hum Gene Ther 10, 2337-2346.
Loh, Y. H., W. Zhang, X. Chen, J. George, and H. H. Ng. 2007. Jmjd1a and Jmjd2c histone H3 Lys 9 demethylases regulate self-renewal in embryonic stem cells. Genes Dev 21:2545-57.
Mankad, A., Taniguchi, T., Cox, B., Akkari, Y., Rathbun, R. K., Lucas, L., Bagby, G., Olson, S., D'Andrea, A., and Grompe, M. (2006). Natural gene therapy in monozygotic twins with Fanconi anemia. Blood 107, 3084-3090.
Maruggi, G., Porcellini, S., Facchini, G., Perna, S. K., Cattoglio, C., Sartori, D., Ambrosi, A., Schambach, A., Baum, C., Bonini, C., et al. (2009). Transcriptional enhancers induce insertional gene deregulation independently from the vector type and design. Mol Ther 17, 851-856.
McMahon, J. M., Signori, E., Wells, K. E., Fazio, V. M., and Wells, D. J. (2001). Optimisation of electrotransfer of plasmid into skeletal muscle by pretreatment with hyaluronidase—increased expression with reduced muscle damage. Gene Ther 8, 1264-1270.
Meneghini, M. D., M. Wu, and H. D. Madhani. 2003. Conserved histone variant H2A.Z protects euchromatin from the ectopic spread of silent heterochromatin. Cell 112:725-36.
Metais, J. Y., and Dunbar, C. E. (2008). The MDS1-EVI1 gene complex as a retrovirus integration site: impact on behavior of hematopoietic cells and implications for gene therapy. Mol Ther 16, 439-449.
Michishita, E., R. A. McCord, E. Berber, M. Kioi, H. Padilla-Nash, M. Damian, P. Cheung, R. Kusumoto, T. L. Kawahara, J. C. Barrett, H. Y. Chang, V. A. Bohr, T. Ried, O. Gozani, and K. F. Chua. 2008. SIRT6 is a histone H3 lysine 9 deacetylase that modulates telomeric chromatin. Nature 452:492-6.
Mito, Y., J. G. Henikoff, and S. Henikoff. 2007. Histone replacement marks the boundaries of cis-regulatory domains. Science 315:1408-11.
Modin, C., Pedersen, F. S., and Duch, M. (2000). Lack of shielding of primer binding site silencer-mediated repression of an internal promoter in a retrovirus vector by the putative insulators scs, BEAD-1, and HS4. J Virol 74, 11697-11707.
Modlich, U., Bohne, J., Schmidt, M., von Kalle, C., Knoss, S., Schambach, A., and Baum, C. (2006). Cell-culture assays reveal the importance of retroviral vector design for insertional genotoxicity. Blood 108, 2545-2553.
Modlich, U., Navarro, S., Zychlinski, D., Maetzig, T., Knoess, S., Brugman, M. H., Schambach, A., Charrier, S., Galy, A., Thrasher, A. J., et al. (2009). Insertional Transformation of Hematopoietic Cells by Self-inactivating Lentiviral and Gammaretroviral Vectors. Mol Ther.
Modlich, U., Schambach, A., Brugman, M. H., Wicke, D.C., Knoess, S., Li, Z., Maetzig, T., Rudolph, C., Schlegelberger, B., and Baum, C. (2008). Leukemia induction after a single retroviral vector insertion in Evi1 or Prdm16. Leukemia 22, 1519-1528.
Montini, E., Cesana, D., Schmidt, M., Sanvito, F., Ponzoni, M., Bartholomae, C., Sergi Sergi, L., Benedicenti, F., Ambrosi, A., Di Serio, C., et al. (2006). Hematopoietic stem cell gene transfer in a tumor-prone mouse model uncovers low genotoxicity of lentiviral vector integration. Nat Biotechnol 24, 687-696.
Moon, A. M., and Ley, T. J. (1990). Conservation of the primary structure, organization, and function of the human and mouse beta-globin locus-activating regions. Proc Natl Acad Sci USA 87, 7693-7697.
Muller, H. P., and Varmus, H. E. (1994). DNA bending creates favored sites for retroviral integration: an explanation for preferred insertion sites in nucleosomes. Embo J 13, 4704-4714.
Muller, K., and Mermod, N. (2000). The histone-interacting domain of nuclear factor I activates simian virus 40 DNA replication in vivo. J Biol Chem 275, 1645-1650.
Nielsen, T. T., Jakobsson, J., Rosenqvist, N., and Lundberg, C. (2009). Incorporating double copies of a chromatin insulator into lentiviral vectors results in less viral integrants. BMC Biotechnol 9, 13.
Nishino, T., Tubb, J., and Emery, D. W. (2006). Partial correction of murine beta-thalassemia with a gammaretrovirus vector for human gamma-globin. Blood Cells Mol Dis 37, 1-7.
Noguchi, M., Nakamura, Y., Russell, S. M., Ziegler, S. F., Tsang, M., Cao, X., and Leonard, W. J. (1993). Interleukin-2 receptor gamma chain: a functional component of the interleukin-7 receptor. Science 262, 1877-1880.
Pankiewicz, R., Karlen, Y., Imhof, M. O., and Mermod, N. (2005). Reversal of the silencing of tetracycline-controlled genes requires the coordinate action of distinctly acting transcription factors. J Gene Med 7, 117-132.
Pedram, M., C. N. Sprung, Q. Gao, A. W. Lo, G. E. Reynolds, and J. P. Murnane. 2006. Telomere position effect and silencing of transgenes near telomeres in the mouse. Mol Cell Biol 26:1865-78.
Perrini, B., L. Piacentini, L. Fanti, F. Altieri, S. Chichiarelli, M. Berloco, C. Turano, A. Ferraro, and S. Pimpinelli. 2004. HP1 controls telomere capping, telomere elongation, and telomere silencing by two different mechanisms in Drosophila. Mol Cell 15:467-76.
Peters, A. H., D. O'Carroll, H. Scherthan, K. Mechtler, S. Sauer, C. Schofer, K. Weipoltshammer, M. Pagani, M. Lachner, A. Kohlmaier, S. Opravil, M. Doyle, M. Sibilia, and T. Jenuwein. 2001. Loss of the Suv39h histone methyltransferases impairs mammalian heterochromatin and genome stability. Cell 107:323-37.
Phillips, J. E., and Corces, V. G. (2009). CTCF: master weaver of the genome. Cell 137, 1194-1211.
Pike-Overzet, K., de Ridder, D., Weerkamp, F., Baert, M. R., Verstegen, M. M., Brugman, M. H., Howe, S. J., Reinders, M. J., Thrasher, A. J., Wagemaker, G., et al. (2006). Gene therapy: is IL2RG oncogenic in T-cell development? Nature 443, E5; discussion E6-7.
Puthenveetil, G., Scholes, J., Carbonell, D., Qureshi, N., Xia, P., Zeng, L., Li, S., Yu, Y., Hiti, A. L., Yee, J. K., and Malik, P. (2004). Successful correction of the human beta-thalassemia major phenotype using a lentiviral vector. Blood 104, 3445-3453.
Qasim, W., Gaspar, H. B., and Thrasher, A. J. (2004). Gene therapy for severe combined immune deficiency. Expert Rev Mol Med 2004, 1-15.
Qiao, J., Diaz, It M., and Vile, R. G. (2004). Success for gene therapy: render unto Caesar that which is Caesar's. Genome Biol 5, 237.
Ramezani, A., Hawley, T. S., and Hawley, R. G. (2003). Performance- and safety-enhanced lentiviral vectors containing the human interferon-beta scaffold attachment region and the chicken beta-globin insulator. Blood 101, 4717-4724.
Recillas-Targa, F., Bell, A. C., and Felsenfeld, G. (1999). Positional enhancer-blocking activity of the chicken beta-globin insulator in transiently transfected cells. Proc Natl Acad Sci USA 96, 14354-14359.
Reitman, M., and Felsenfeld, G. (1988). Mutational analysis of the chicken beta-globin enhancer reveals two positive-acting domains. Proc Natl Acad Sci USA 85, 6267-6271.
Rincon-Arano, H., M. Furlan-Magaril, and F. Recillas-Targa. 2007. Protection against telomeric position effects by the chicken cHS4 beta-globin insulator. Proc Natl Acad Sci USA 104:14044-9.
Rivella, S., Callegari, J. A., May, C., Tan, C. W., and Sadelain, M. (2000). The cHS4 insulator increases the probability of retroviral expression at random chromosomal integration sites. J Virol 74, 4679-4687.
Roulet E, Busso S, Camargo AA, Simpson AJG, Mermod N, and Bucher P. (2002). High resolution and characterization of transcription factor DNA binding specificity by coupled protocols for SAGE-assisted SELEX and computer modeling. Nature Biotechnol., 20, 831-835
Roulet, E., Busso, S., Camargo, A. A., Simpson, A. J., Mermod, N., and Bucher, P. (2002). High-throughput SELEX SAGE method for quantitative modeling of transcription-factor binding sites. Nat Biotechnol 20, 831-835.
Rupp, R. A., Kruse, U., Multhaup, G., Gobel, U., Beyreuther, K., and Sippel, A. E. (1990). Chicken NFI/TGGCA proteins are encoded by at least three independent genes: NFI-A, NFI-B and NFI-C with homologues in mammalian genomes. Nucleic Acids Res 18, 2607-2616.
Santoro, C., N. Mermod, P. C. Andrews, and R. Tjian. 1988. A family of human CCAAT-box-binding proteins active in transcription and DNA replication: cloning and expression of multiple cDNAs. Nature 334:218-24.
Schambach, A., Bohne, J., Chandra, S., Will, E., Margison, G. P., Williams, D. A., and Baum, C. (2006a). Equal potency of gammaretroviral and lentiviral SIN vectors for expression of O6-methylguanine-DNA methyltransferase in hematopoietic cells. Mol Ther 13, 391-400.
Schambach, A., Mueller, D., Galla, M., Verstegen, M. M., Wagemaker, G., Loew, R., Baum, C., and Bohne, J. (2006b). Overcoming promoter competition in packaging cells improves production of self-inactivating retroviral vectors. Gene Ther 13, 1524-1533.
Schotta, G., M. Lachner, K. Sarma, A. Ebert, R. Sengupta, G. Reuter, D. Reinberg, and T. Jenuwein. 2004. A silencing pathway to induce H3-K9 and H4-K20 trimethylation at constitutive heterochromatin. Genes Dev 18:1251-62.
Schroder, A. R., Shinn, P., Chen, H., Berry, C., Ecker, J. R., and Bushman, F. (2002). HIV-1 integration in the human genome favors active genes and local hotspots. Cell 110, 521-529.
Splinter, E., Heath, H., Kooren, J., Palstra, R. J., Klous, P., Grosveld, F., Galjart, N., and de Laat, W. (2006). CTCF mediates long-range chromatin looping and local histone modification in the beta-globin locus. Genes Dev 20, 2349-2354.
Stocking, C., Bergholz, U., Friel, J., Klingler, K., Wagener, T., Starke, C., Kitamura, T., Miyajima, A., and Ostertag, W. (1993). Distinct classes of factor-independent mutants can be isolated after retroviral mutagenesis of a human myeloid stem cell line. Growth Factors 8, 197-209.

Sugamura, K., Asao, H., Kondo, M., Tanaka, N., Ishii, N., Ohbo, K., Nakamura, M., and Takeshita, T. (1996). The interleukin-2 receptor gamma chain: its role in the multiple cytokine receptor complexes and T cell development in XSCID. Annu Rev Immunol 14, 179-205.
Sun, F. L., and Elgin, S.C. (1999). Putting boundaries on silence. Cell 99, 459-462.
Tarapore, P., Richmond, C., Zheng, G., Cohen, S. B., Kelder, B., Kopchick, J., Kruse, U., Sippel, A. E., Colmenares, C., and Stavnezer, E. (1997). DNA binding and transcriptional activation by the Ski oncoprotein mediated by interaction with NFI. Nucleic Acids Res 25, 3895-3903.
Thrasher, A. J., Gaspar, H. B., Baum, C., Modlich, U., Schambach, A., Candotti, F., Otsu, M., Sorrentino, B., Scobie, L., Cameron, E., et al. (2006). Gene therapy: X-SCID transgene leukaemogenicity. Nature 443, E5-6; discussion E6-7.
Tolhuis, B., Palstra, R. J., Splinter, E., Grosveld, F., and de Laat, W. (2002). Looping and interaction between hypersensitive sites in the active beta-globin locus. Mol Cell 10, 1453-1465.
Tsukada, Y., and Y. Zhang. 2006. Purification of histone demethylases from HeLa cells. Methods 40:318-26.
Urbinati, F., Arumugam, P., Higashimoto, T., Perumbeti, A., Mitts, K., Xia, P., and Malik, P. (2009). Mechanism of reduction in titers from lentivirus vectors carrying large inserts in the 3′LTR. Mol Ther 17, 1527-1536.
Vakoc, C. R., S. A. Mandat, B. A. Olenchock, and G. A. Blobel. 2005. Histone H3 lysine 9 methylation and HPlgamma are associated with transcription elongation through mammalian chromatin. Mol Cell 19:381-91.
Verma, I. M., and Somia, N. (1997). Gene therapy—promises, problems and prospects. Nature 389, 239-242.
von Kalle, C., Fehse, B., Layh-Schmitt, G., Schmidt, M., Kelly, P., and Baum, C. (2004). Stem cell clonality and genotoxicity in hematopoietic cells: gene activation side effects should be avoidable. Semin Hematol 41, 303-318.
West, A. G., Gaszner, M., and Felsenfeld, G. (2002). Insulators: many functions, many mechanisms. Genes Dev 16, 271-288.
West, A. G., Huang, S., Gaszner, M., Litt, M. D., and Felsenfeld, G. (2004). Recruitment of histone modifications by USF proteins at a vertebrate barrier element. Mol Cell 16, 453-463.
Woods, N. B., Bottero, V., Schmidt, M., von Kalle, C., and Verma, I. M. (2006). Gene therapy: therapeutic gene causing lymphoma. Nature 440, 1123.
Wu, X., Li, Y., Crise, B., and Burgess, S. M. (2003). Transcription start regions in the human genome are favored targets for MLV integration. Science 300, 1749-1751.
Yamada, Y., Warren, A. J., Dobson, C., Forster, A., Pannell, R., and Rabbitts, T. H. (1998). The T cell leukemia LIM protein Lmo2 is necessary for adult mouse hematopoiesis. Proc Natl Acad Sci USA 95, 3890-3895.
Yannaki, E., Tubb, J., Aker, M., Stamatoyannopoulos, G., and Emery, D. W. (2002). Topological constraints governing the use of the chicken HS4 chromatin insulator in oncoretrovirus vectors. Mol Ther 5, 589-598.
Yao, S., Osborne, C. S., Bharadwaj, R. R., Pasceri, P., Sukonnik, T., Pannell, D., Recillas-Targa, F., West, A. G., and Ellis, J. (2003). Retrovirus silencer blocking by the cHS4 insulator is CTCF independent. Nucleic Acids Res 31, 5317-5323.
Ye, J. Z., D. Hockemeyer, A. N. Krutchinsky, D. Loayza, S. M. Hooper, B. T. Chait, and T. de Lange. 2004. POT1-interacting protein PIP1: a telomere length regulator that recruits POT1 to the TIN2/TRF1 complex. Genes Dev 18:1649-54.
Zaiss, A. K., Son, S., and Chang, L. J. (2002). RNA 3′ readthrough of oncoretrovirus and lentivirus: implications for vector safety and efficacy. J Virol 76, 7209-7219.
Zayed, H., McIvor, R. S., Wiest, D. L., and Blazar, B. R. (2006). In vitro functional correction of the mutation responsible for murine severe combined immune deficiency by small fragment homologous replacement. Hum Gene Ther 17, 158-166.
Zhang, H., D. N. Roberts, and B. R. Cairns. 2005. Genome-wide dynamics of Htz1, a histone H2A variant that poises repressed/basal promoters for activation through histone loss. Cell 123:219-31.
Zlatanova, J., and Caiafa, P. (2009a). CCCTC-binding factor: to loop or to bridge. Cell Mol Life Sci 66, 1647-1660.
Zlatanova, J., and Caiafa, P. (2009b). CTCF and its protein partners: divide and rule? J Cell Sci 122, 1275-1284.
Zychlinski, D., Schambach, A., Modlich, U., Maetzig, T., Meyer, J., Grassman, E., Mishra, A., and Baum, C. (2008). Physiological Promoters Reduce the Genotoxic Risk of Integrating Gene Vectors. Mol Ther.

1. An isolated DNA molecule having insulator capable of enhancer-blocking activity wherein, it comprises at least one binding site sequence for a CTF/NF-1 protein.

2. (canceled)

3. The isolated DNA molecule of claim 1, wherein CTF/NF-1 is a CTF-1.

4. (canceled)

5. The isolated DNA molecule of claim 1, wherein said CTF/NF-1 binding site comprises a sequence selected from the group consisting of SEQ ID No 1, SEQ ID No 13, SEQ ID No 14, SEQ ID No 15, SEQ ID No 16, SEQ ID No 17, SEQ ID No 18, SEQ ID No 19, SEQ ID No 20, SEQ ID No 22, SEQ ID No 23, SEQ ID No 24, SEQ ID No 25, SEQ ID No 26, SEQ ID No 27, SEQ ID No 28, SEQ ID No 29 and/or combinations thereof.

6. The isolated DNA molecule of claim 5, wherein said at least one binding site sequence for the CTF/NF-1 protein further comprises a binding sequence element selected from the group consisting of USF1/2, BRCA1, Oct1, Sp1, Ars1, SatB1, CREB, C/EBP, NMP4, Hox, Gsh, Fast1 and/or combinations thereof.

7. (canceled)

8. The isolated DNA molecule of claim 1, wherein said isolated DNA molecule is functionally linked to a U3-deleted LTR.

9. An expression vector comprising:
(a) at least one copy of the isolated DNA molecule of claim 1,
(b) a promoter domain;
(c) a gene of interest operably linked to the promoter domain, and
(d) an enhancer domain 5′ of the promoter domain.

10. (canceled)

11. (canceled)

12. The expression vector of claim 9, wherein it comprises between one or more copies of said isolated DNA molecule.

13. The expression vector of claim 12, wherein it comprises between one and twelve copies of said isolated DNA molecule.

14. (canceled)

15. (canceled)

16. (canceled)

17. The expression vector of claim 9, wherein said enhancer domain is selected from the group consisting of viral enhancers, eukaryotic enhancers, preferably mammalian enhancers.

18. A method for detecting a DNA molecule having insulator capable of enhancer-blocking activity comprising the steps of:
a) providing an expression vector wherein said isolated DNA molecule according to claim 1 is positioned between a potent enhancer and a promoter domain operably linked to a reporter gene,
b) introducing the expression vector of step a) into a cell,
c) quantifying the expression of the reporter gene, and
d) correlating said reporter gene expression to potential insulator or boundary properties of said DNA molecule.

19. The method of claim 18, wherein the potent enhancer is a retroviral enhancer.

20. The method of claim 18, wherein the reporter gene encodes for a fluorescent protein.

21. The method of claim 18, wherein, the expression vector comprises an additional gene which is not submitted to the activity of the insulator.

22. (canceled)

23. A method for treating a subject diagnosed with a genetic disease, the method comprising administering an expression vector of claim 9, so as to complement the genetic deficiency.

24. A host cell comprising the isolated DNA molecule of claim 1.

25. The host cell of claim 24, consisting of a stem cell, a cultured cell or an ex vivo transduced cell.

26. The use of the isolated DNA molecules of claim 1 as insulator capable of enhancer-blocking activity.

27. A mammalian cell stably transfected with the isolated DNA molecule of claim 1.

28. A mammalian cell stably transfected with the isolated DNA molecule comprising at least one copy of the expression vector of claim 9.

29. A host cell comprising the isolated DNA molecule comprising at least one copy of the expression vector of claim 9.


SEQUENCE LISTING (34 sequences)

SEQ ID NO: 1 - DNA, 20 bp, Artificial Sequence
  CTF/NF1 binding site from the adenovirus type II origin of replication
ttggcaacgtgccataagca

SEQ ID NO: 2 - DNA, 42 bp, Artificial Sequence
  CTCF binding site from the footprint II (FII) of the cHS4 insulator
cccagggatgtaattacgtccctcccccgctagggggcagca

SEQ ID NO: 3 - DNA, 39 bp, Artificial Sequence
  CTCF binding site  from human T cell receptor alpha/delta locus BEAD A
cccaggcctgcactgccgcctgccggcaggggtccagtc

SEQ ID NO: 4 - DNA, 18 bp, Artificial Sequence
  complementary to the murine GAPDH cDNA for quantitative PCR
cgcccccggtttctataa

SEQ ID NO: 5 - DNA, 21 bp, Artificial Sequence
  complementary to the murine GAPDH cDNA for quantitative PCR
actgtcgaacaggaggagcag

SEQ ID NO: 6 - DNA, 20 bp, Artificial Sequence
  complementary to the EGFP cDNA for quantitative PCR
agcaaagaccccaccgagaa

SEQ ID NO: 7 - DNA, 15 bp, Artificial Sequence
  complementary to the EGFP cDNA for quantitative PCR
ggcggcggtcacgaa

SEQ ID NO: 8 - DNA, 20 bp, Artificial Sequence
  complementary to the dsRED cDNA for quantitative PCR
ttccagtacgggtccaaggt

SEQ ID NO: 9 - DNA, 23 bp, Artificial Sequence
  complementary to the dsRED cDNA for quantitative PCR
ggaaggacagcttcttgtagtcg

SEQ ID NO: 10 - DNA, 6 bp, Artificial Sequence
  Murine telomeric repeat
ttaggg

SEQ ID NO: 11 - DNA, 472 bp, Artificial Sequence
  Consensus binding sites for CTCF (12copies)
gcgatgccgccccctggtggccagtaatcgcaaggctaagtaatcactgccccctggtggccgccagtctgatacgcgttttacaaccgccccctggtggccgtgggagacatctagtgcacgagagtgccccctggtggccaaaccgtagcctaggcatattgtactgccccctggtggccggcaatatggctagcgatgactcggcgccccctggtggccactacgttctagtggcgatgccgccccctggtggccagtaatcgcaaggctaagtaatcactgccccctggtggccgccagtctgatacgcgttttacaaccgccccctggtggccgtgggagacatctagtgcacgagagtgccccctggtggccaaaccgtagcctaggcatattgtactgccccctggtggccggcaatatggctagcgatgactcggcgccccctggtggccactacgttctagtg

SEQ ID NO: 12 - DNA, 236 bp, Artificial Sequence
  CTCF consensus binding sites
gcgatgccgccccctggtggccagtaatcgcaaggctaagtaatcactgccccctggtggccgccagtctgatacgcgttttacaaccgccccctggtggccgtgggagacatctagtgcacgagagtgccccctggtggccaaaccgtagcctaggcatattgtactgccccctggtggccggcaatatggctagcgatgactcggcgccccctggtggccactacgttctagtg

SEQ ID NO: 13 - DNA, 24 bp, Artificial Sequence
  1xCTF/NF1 from adenovirus type II
actagttggcaacgtgccataagc

SEQ ID NO: 14 - DNA, 164 bp, Artificial Sequence
  7xCTF/NF1 from adenovirus type II
atcgataagcttgcattggcaacgtgccataagcattggcaacgtgccataagcattggcaacgtgccataagcattggcaacgtgccataagcattggcaacgtgccataagcattggcaacgtgccataagcattggcaacgtgccataagcggggggatcc

SEQ ID NO: 15 - DNA, 70 bp, Artificial Sequence
  3xCTF/NF1 from adenovirus type II but combination of sites and flanking sequences artificial
ctagattggcaatctgccatgctagcttgtttggcagactgccatcctaggtcagttggcatgatgccat

SEQ ID NO: 16 - DNA, 170 bp, Artificial Sequence
  7xCTF/NF1 from adenovirus type II but combination of sites and flanking sequences artificial
ctagattggcaatctgccatgctagcttgtttggcagactgccatcctaggtcagttggcatgatgccatctagatgtcattggcaaactgccattgcatctgtattggcagtatgccatgttactcttgttggcactgtgccatgatacagatattggcaccttgccat

SEQ ID NO: 17 - DNA, 60 bp, Artificial Sequence
  3xCTF/NF1
ctagattggcaatctgccaagctgtttggcagactgccaacccagttggcatgatgccaa

SEQ ID NO: 18 - DNA, 140 bp, Artificial Sequence
  7xCTF/NF1
ctagattggcaatctgccaagctgtttggcagactgccaacccagttggcatgatgccaactagattggcaaactgccaatggtattggcagtatgccaagtttgttggcactgtgccaagaatattggcaccttgccaa

SEQ ID NO: 19 - DNA, 70 bp, Artificial Sequence
  3xCTF/NF1
ctagattggcaatctgccaagctagcttgtttggcagactgccaacctaggtcagttggcatgatgccaa

SEQ ID NO: 20 - DNA, 170 bp, Artificial Sequence
  7xCTF/NF1
ctagattggcaatctgccaagctagcttgtttggcagactgccaacctaggtcagttggcatgatgccaactagatgtcattggcaaactgccaatgcatctgtattggcagtatgccaagttactcttgttggcactgtgccaagatacagatattggcaccttgccaa

SEQ ID NO: 21 - DNA, 14 bp, Artificial Sequence
  1x CTCF consensus sequence
gccccctggtggcc

SEQ ID NO: 22 - DNA, 101 bp, Artificial Sequence
  4 CTF/NF1 binding sites
actagatgtcattggcaaactgccaatgcatctgtattggcagtatgccaagttactcttgttggcactgtgccaagatacagatattggcaccttgccaa

SEQ ID NO: 23 - DNA, 80 bp, Artificial Sequence
  4 CTF/NF1 binding sites
actagattggcaaactgccaatggtattggcagtatgccaagtttgttggcactgtgccaagaatattggcaccttgcca

SEQ ID NO: 24 - DNA, 20 bp, Artificial Sequence
  1 CTF/NF1 binding site
attggcaacgtgccataagc

SEQ ID NO: 25 - DNA, 81 bp, Artificial Sequence
  3 CTF/NF1 binding sites
taagcttgcattggcaacgtgccataagcattggcaacgtgccataagcattggcaacgtgccataagcgaattgggggat

SEQ ID NO: 26 - DNA, 81 bp, Artificial Sequence
  3 CTF/NF1 binding sites
atcccccaattcgcttatggcacgttgccaatgcttatggcacgttgccaatgcttatggcacgttgccaatgcaagctta

SEQ ID NO: 27 - DNA, 101 bp, Artificial Sequence
  4 CTF/NF1 binding sites
atcccccaattcgcttatggcacgttgccaatgcttatggcacgttgccaatgcttatggcacgttgccaatgcttatggcacgttgccaatgcaagctta

SEQ ID NO: 28 - DNA, 100 bp, Artificial Sequence
  4 CTF/NF1 binding sites
atgtcattggcaaactgccattgcatctgtattggcagtatgccatgttactcttgttggcactgtgccatgatacagatattggcaccttgccatctag

SEQ ID NO: 29 - DNA, 15 bp, Artificial Sequence
  1 CTF/NF1 consensus
ttggcnnnnngccaa

SEQ ID NO: 30 - DNA, 14 bp, Artificial Sequence
  1x CTCF consensus sequence (complentary to SEQ ID No. 21)
ggccaccagggggc

SEQ ID NO: 31 - DNA, 19 bp, Artificial Sequence
  synthesized
gaggagttgtggcccttgt

SEQ ID NO: 32 - DNA, 20 bp, Artificial Sequence
  synthesized
tgacaggtggtggcaatgcc

SEQ ID NO: 33 - DNA, 22 bp, Artificial Sequence
  synthesized
ggtttcaatgtcccgtatcctt

SEQ ID NO: 34 - DNA, 20 bp, Artificial Sequence
  synthesized
ctttgccccagtcccagtta